SEQUENCE LISTING (5 sequences)

SEQ ID NO: 1 - DNA, 11354 bp, Artificial Sequence
  Recombinant lyssavirus DNA
atggatgccgacaagattgtattcaaagtcaataatcaggtggtctctttgaagcctgagattatcgtggatcaatatgagtacaagtaccctgccatcaaagatttgaaaaagccctgtataaccctaggaaaggctcccgatttaaataaagcatacaagtcagttttgtcaggcatgagcgccgccaaacttaatcctgacgatgtatgttcctatttggcagcggcaatgcagttttttgaggggacatgtccggaagactggaccagctatggaattgtgattgcacgaaaaggagataagatcaccccaggttctctggtggagataaaacgtactgatgtagaagggaattgggctctgacaggaggcatggaactgacaagagaccccactgtccctgagcatgcgtccttagtcggtcttctcttgagtctgtataggttgagcaaaatatccgggcaaaacactggtaactataagacaaacattgcagacaggatagagcagatttttgagacagccccttttgttaaaatcgtggaacaccatactctaatgacaactcacaaaatgtgtgctaattggagtactataccaaacttcagatttttggccggaacctatgacatgtttttctcccggattgagcatctatattcagcaatcagagtgggcacagttgtcactgcttatgaagactgttcaggactggtatcatttactgggttcataaaacaaatcaatctcaccgctagagaggcaatactatatttcttccacaagaactttgaggaagagataagaagaatgtttgagccagggcaggagacagctgttcctcactcttatttcatccacttccgttcactaggcttgagtgggaaatctccttattcatcaaatgctgttggtcacgtgttcaatctcattcactttgtaggatgctatatgggtcaagtcagatccctaaatgcaacggttattgctgcatgtgctcctcatgaaatgtctgttctagggggctatctgggagaggaattcttcgggaaagggacatttgaaagaagattcttcagagatgagaaagaacttcaagaatacgaggcggctgaactgacaaagactgacgtagcactggcagatgatggaactgtcaactctgacgacgaggactacttttcaggtgaaaccagaagtccggaggctgtttatactcgaatcatgatgaatggaggtcgactaaagagatctcacatacggagatatgtctcagtcagttccaatcatcaagcccgtccaaactcattcgccgagtttctaaacaagacatattcgagtgactcataacatgaaaaaaactaacacccctcccgtacggccgccaccatggtccctcaggctctgctgttcgtcccactgctggtcttccctctgtgctttggcaagttccctatctacactattcccgacaagctgggcccctggtctcctatcgatattcaccatctgagttgccctaacaatctggtggtcgaggacgaagggtgtaccaacctgagcggcttctcctacatggagctgaaagtgggatatatcctggctattaaggtcaacgggttcacatgcactggcgtggtcaccgaggcagaaacctacacaaattttgtgggctatgtcaccacaactttcaagaggaaacactttagaccaacacccgacgcctgtcgcgccgcttacaactggaagatggctggcgatccacgatatgaggaatctctgcacaatccttacccagactatagatggctgcggactgtgaagaccacaaaagagtccctggtcatcatttccccttctgtcgcagacctggatccatacgatagatctctgcacagtcgggtgtttccctccggaaagtgctctggggtggctgtcagctccacttactgtagcaccaaccatgattatacaatctggatgccagagaatcccaggctggggatgagctgcgacattttcacaaattcccgcggcaagcgagcctcaaaaggaagcgagacttgtgggtttgtggacgaaaggggactgtataagagcctgaaaggggcttgcaagctgaaactgtgcggcgtgctgggactgagactgatggatggcacctgggtcagtatgcagacatcaaacgagactaagtggtgcccccctgacaaactggtgaatctgcacgacttcaggagcgacgagatcgaacatctggtggtcgaggaactggtgcgaaaaagggaggaatgtctggatgccctggagtccatcatgactaccaagagcgtgagcttcaggaggctgtctcacctgcgaaagctggtgcccggcttcggcaaagcctacaccatctttaacaagacactgatggaagcagacgcccattataaatcagtggagacctggaatgaaattctgccaagcaagggctgcctgcgggtgggcggacgctgtcacccacatgtgaacggcgtcttctttaatggaatcattctggggcccgacggcaacgtgctgatccctgagatgcagtctagtctgctgcagcagcacatggagctgctggaatcaagcgtgattcctctggtccatccactggcagatccctccacagtgttcaaagacggagatgaggccgaagactttgtggaagtccacctgcctgatgtgcataaccaggtgtctggcgtcgacctgggactgccaaattggggcaagtacgtgctgctgagtgctggagcactgactgccctgatgctgatcattttcctgatgacctgctgtcggcgcgtgaacagaagtgagcccactcagcacaatctgcgaggaaccgggagagaagtgtcagtcacacctcagagcgggaaaatcattagtagttgggaatcacataaaagcgggggcgagaccaggctgtgagctagccatgaaaaaaactaacacccctcctttcgaaccatcccaaacatgagcaagatctttgtcaatcctagtgctattagagccggtctggccgatcttgagatggctgaagaaactgttgatctgatcaatagaaatatcgaagacaatcaggctcatctccaaggggaacccatagaggtggacaatctccctgaggatatggggcgacttcacctggatgatggaaaatcgcccaaccatggtgagatagccaaggtgggagaaggcaagtatcgagaggactttcagatggatgaaggagaggatcctagcttcctgttccagtcatacctggaaaatgttggagtccaaatagtcagacaaatgaggtcaggagagagatttctcaagatatggtcacagaccgtagaagagattatatcctatgtcgcggtcaactttcccaaccctccaggaaagtcttcagaggataaatcaacccagactactggccgagagctcaagaaggagacaacacccactccttctcagagagaaagccaatcatcgaaagccaggatggcggctcaaattgcttctggccctccagcccttgaatggtcggctaccaatgaagaggatgatctatcagtggaggctgagatcgctcaccagattgcagaaagtttctccaaaaaatataagtttccctctcgatcctcagggatactcttgtataattttgagcaattgaaaatgaaccttgatgatatagttaaagaggcaaaaaatgtaccaggtgtgacccgtttagcccatgacgggtccaaactccccctaagatgtgtactgggatgggtcgctttggccaactctaagaaattccagttgttagtcgaatccgacaagctgagtaaaatcatgcaagatgacttgaatcgctatacatcttgctaaccgaacctctcccctcagtccctctagacaataaaatccgagatgtcccaaagtcaacatgaaaaaaacaggcaacaccactgataaaatgaacctcctacgtaagatagtgaaaaaccgcagggacgaggacactcaaaaatcctctcccgcgtcagcccctctggatgacgatgacttgtggcttccaccccctgaatacgtcccgctgaaagaacttacaggcaagaagaacatgaggaacttttgtatcaacggaagggttaaagtgtgtagcccgaatggttactcgttcaggatcctgcggcacattctgaaatcattcgacgagatatattctgggaatcataggatgatcgggttagtcaaagtggttattggactggctttgtcaggatctccagtccctgagggcctgaactgggtatacaaattgaggagaacctttatcttccagtgggctgattccaggggccctcttgaaggggaggagttggaatactctcaggagatcacttgggatgatgatactgagttcgtcggattgcaaataagagtgattgcaaaacagtgtcatatccagggcagagtctggtgtatcaacatgaacccgagagcatgtcaactatggtctgacatgtctcttcagacacaaaggtccgaagaggacaaagattcctctctgcttctagaataatcagattatatcccgcaaatttatcacttgtttacctctggaggagagaacatatgggctcaactccaacccttgggagcaatataacaaaaaacatgttatggtgccattaaaccgctgcatttcatcaaagtcaagttgattacctttacattttgatcctcttggatgtgaaaaaaactattaacatccctcaaaagaccccggtaacgtcctttcaacgatccaagtccatgaaaaaaactaacacccctcccgtacctagcttataaagtgctgggtcatctaagcttttcagtcgagaaaaaaacattagatcagaagaacaactggcaacacttctcaacctgagacttacttcaagatgctcgatcctggagaggtctatgatgaccctattgacccaatcgagttagaggctgaacccagaggaacccccattgtccccaacatcttgaggaactctgactacaatctcaactctcctttgatagaagatcctgctagactaatgttagaatggttaaaaacagggaatagaccttatcggatgactctaacagacaattgctccaggtctttcagagttttgaaagattatttcaagaaggtagatttgggttctctcaaggtgggcggaatggctgcacagtcaatgatttctctctggttatatggtgcccactctgaatccaacaggagccggagatgtataacagacttggcccatttctattccaagtcgtcccccatagagaagctgttgaatctcacgctaggaaatagagggctgagaatccccccagagggagtgttaagttgccttgagagggttgattatgataatgcatttggaaggtatcttgccaacacgtattcctcttacttgttcttccatgtaatcaccttatacatgaacgccctagactgggatgaagaaaagaccatcctagcattatggaaagatttaacctcagtggacatcgggaaggacttggtaaagttcaaagaccaaatatggggactgctgatcgtgacaaaggactttgtttactcccaaagttccaattgtctttttgacagaaactacacacttatgctaaaagatcttttcttgtctcgcttcaactccttaatggtcttgctctctcccccagagccccgatactcagatgacttgatatctcaactatgccagctgtacattgctggggatcaagtcttgtctatgtgtggaaactccggctatgaagtcatcaaaatattggagccatatgtcgtgaatagtttagtccagagagcagaaaagtttaggcctctcattcattccttgggagactttcctgtatttataaaagacaaggtaagtcaacttgaagagacgttcggtccctgtgcaagaaggttctttagggctctggatcaattcgacaacatacatgacttggtttttgtgtttggctgttacaggcattgggggcacccatatatagattatcgaaagggtctgtcaaaactatatgatcaggttcaccttaaaaaaatgatagataagtcctaccaggagtgcttagcaagcgacctagccaggaggatccttagatggggttttgataagtactccaagtggtatctggattcaagattcctagcccgagaccaccccttgactccttatatcaaaacccaaacatggccacccaaacatattgtagacttggtgggggatacatggcacaagctcccgatcacgcagatctttgagattcctgaatcaatggatccgtcagaaatattggatgacaaatcacattctttcaccagaacgagactagcttcttggctgtcagaaaaccgaggggggcctgttcctagcgaaaaagttattatcacggccctgtctaagccgcctgtcaatccccgagagtttctgaggtctatagacctcggaggattgccagatgaagacttgataattggcctcaagccaaaggaacgggaattgaagattgaaggtcgattctttgctctaatgtcatggaatctaagattgtattttgtcatcactgaaaaactcttggccaactacatcttgccactttttgacgcgctgactatgacagacaacctgaacaaggtgtttaaaaagctgatcgacagggtcaccgggcaagggcttttggactattcaagggtcacatatgcatttcacctggactatgaaaagtggaacaaccatcaaagattagagtcaacagaggatgtattttctgtcctagatcaagtgtttggattgaagagagtgttttctagaacacacgagttttttcaaaaggcctggatctattattcagacagatcagacctcatcgggttacgggaggatcaaatatactgcttagatgcgtccaacggcccaacctgttggaatggccaggatggcgggctagaaggcttacggcagaagggctggagtctagtcagcttattgatgatagatagagaatctcaaatcaggaacacaagaaccaaaatactagctcaaggagacaaccaggttttatgtccgacatacatgttgtcgccagggctatctcaagaggggctcctctatgaattggagagaatatcaaggaatgcactttcgatatacagagccgtcgaggaaggggcatctaagctagggctgatcatcaagaaagaagagaccatgtgtagttatgacttcctcatctatggaaaaacccctttgtttagaggtaacatattggtgcctgagtccaaaagatgggccagagtctcttgcgtctctaatgaccaaatagtcaacctcgccaatataatgtcgacagtgtccaccaatgcgctaacagtggcacaacactctcaatctttgatcaaaccgatgagggattttctgctcatgtcagtacaggcagtctttcactacctgctatttagcccaatcttaaagggaagagtttacaagattctgagcgctgaaggggagagctttctcctagccatgtcaaggataatctatctagatccttctttgggagggatatctggaatgtccctcggaagattccatatacgacagttctcagaccctgtctctgaagggttatccttctggagagagatctggttaagctcccaagagtcctggattcacgcgttgtgtcaagaggctggaaacccagatcttggagagagaacactcgagagcttcactcgccttctagaagatccgaccaccttaaatatcagaggaggggccagtcctaccattctactcaaggatgcaatcagaaaggctttatatgacgaggtggacaaggtggaaaattcagagtttcgagaggcaatcctgttgtccaagacccatagagataattttatactcttcttaatatctgttgagcctctgtttcctcgatttctcagtgagctattcagttcgtcttttttgggaatccccgagtcaatcattggattgatacaaaactcccgaacgataagaaggcagtttagaaagagtctctcaaaaactttagaagaatccttctacaactcagagatccacgggattagtcggatgacccagacacctcagagggttgggggggtgtggccttgctcttcagagagggcagatctacttagggagatctcttggggaagaaaagtggtaggcacgacagttcctcacccttctgagatgttgggattacttcccaagtcctctatttcttgcacttgtggagcaacaggaggaggcaatcctagagtttctgtatcagtactcccgtcctttgatcagtcatttttttcacgaggccccctaaagggatacttgggctcgtccacctctatgtcgacccagctattccatgcatgggaaaaagtcactaatgttcatgtggtgaagagagctctatcgttaaaagaatctataaactggttcattactagagattccaacttggctcaagctctaattaggaacattatgtctctgacaggccctgatttccctctagaggaggcccctgtcttcaaaaggacggggtcagccttgcataggttcaagtctgccagatacagcgaaggagggtattcttctgtctgcccgaacctcctctctcatatttctgttagtacagacaccatgtctgatttgacccaagacgggaagaactacgatttcatgttccagccattgatgctttatgcacagacatggacatcagagctggtacagagagacacaaggctaagagactctacgtttcattggcacctccgatgcaacaggtgtgtgagacccattgacgacgtgaccctggagacctctcagatcttcgagtttccggatgtgtcgaaaagaatatccagaatggtttctggggctgtgcctcacttccagaggcttcccgatatccgtctgagaccaggagattttgaatctctaagcggtagagaaaagtctcaccatatcggatcagctcaggggctcttatactcaatcttagtggcaattcacgactcaggatacaatgatggaaccatcttccctgtcaacatatacggcaaggtttcccctagagactatttgagagggctcgcaaggggagtattgataggatcctcgatttgcttcttgacaagaatgacaaatatcaatattaatagacctcttgaattggtctcaggggtaatctcatatattctcctgaggctagataaccatccctccttgtacataatgctcagagaaccgtctcttagaggagagatattttctatccctcagaaaatccccgccgcttatccaaccactatgaaagaaggcaacagatcaatcttgtgttatctccaacatgtgctacgctatgagcgagagataatcacggcgtctccagagaatgactggctatggatcttttcagactttagaagtgccaaaatgacgtacctatccctcattacttaccagtctcatcttctactccagagggttgagagaaacctatctaagagtatgagagataacctgcgacaattgagttctttgatgaggcaggtgctgggcgggcacggagaagataccttagagtcagacgacaacattcaacgactgctaaaagactctttacgaaggacaagatgggtggatcaagaggtgcgccatgcagctagaaccatgactggagattacagccccaacaagaaggtgtcccgtaaggtaggatgttcagaatgggtctgctctgctcaacaggttgcagtctctacctcagcaaacccggcccctgtctcggagcttgacataagggccctctctaagaggttccagaaccctttgatctcgggcttgagagtggttcagtgggcaaccggtgctcattataagcttaagcctattctagatgatctcaatgttttcccatctctctgccttgtagttggggacgggtcaggggggatatcaagggcagtcctcaacatgtttccagatgccaagcttgtgttcaacagtcttttagaggtgaatgacctgatggcttccggaacacatccactgcctccttcagcaatcatgaggggaggaaatgatatcgtctccagagtgatagatcttgactcaatctgggaaaaaccgtccgacttgagaaacttggcaacctggaaatacttccagtcagtccaaaagcaggtcaacatgtcctatgacctcattatttgcgatgcagaagttactgacattgcatctatcaaccggatcaccctgttaatgtccgattttgcattgtctatagatggaccactctatttggtcttcaaaacttatgggactatgctagtaaatccaaactacaaggctattcaacacctgtcaagagcgttcccctcggtcacagggtttatcacccaagtaacttcgtctttttcatctgagctctacctccgattctccaaacgagggaagtttttcagagatgctgagtacttgacctcttccacccttcgagaaatgagccttgtgttattcaattgtagcagccccaagagtgagatgcagagagctcgttccttgaactatcaggatcttgtgagaggatttcctgaagaaatcatatcaaatccttacaatgagatgatcataactctgattgacagtgatgtagaatcttttctagtccacaagatggttgatgatcttgagttacagaggggaactctgtctaaagtggctatcattatagccatcatgatagttttctccaacagagtcttcaacgtttccaaacccctaactgacccctcgttctatccaccgtctgatcccaaaatcctgaggcacttcaacatatgttgcagtactatgatgtatctatctactgctttaggtgacgtccctagcttcgcaagacttcacgacctgtataacagacctataacttattacttcagaaagcaagtcattcgagggaacgtttatctatcttggagttggtccaacgacacctcagtgttcaaaagggtagcctgtaattctagcctgagtctgtcatctcactggatcaggttgatttacaagatagtgaagactaccagactcgttggcagcatcaaggatctatccagagaagtggaaagacaccttcataggtacaacaggtggatcaccctagaggatatcagatctagatcatccctactagactacagttgcctgtga

SEQ ID NO: 2 - DNA, 1569 bp, Artificial Sequence
  MOKV glycoprotein DNA
atgaatctcccgtgtttgactgttatcttgatattgtttactaaatactcactgggagaattccctttgtatactataccagagaaaatagagaagtggactccgattgatatgatacacctgtcttgtccgaataatctgctctcccaagaggagggctgcaacgccgagacacctttcacgtactttgagctcaaatcaggctatttggcgcatcagaaggttcaggggtttacctgcactggagtagtgaatgaagcggaaacctataccaactttgtaggatatgttacaacaacgtttaagcggaaacatttcagacccactgtagcagcatgcagggacgcatataattggaaggtgagcggtgaccctcggtatgaggaaagtcttcacaccccctaccctgatagttcctggttgcggaccgtcaccacaaccaaagagagtctgctgataataagtccgtccattgtcgagatggatatttatggccggactcttcacagcccgatgttcccgtcagggacgtgtagcaagctgtacccgtcagtaccatcctgcaagacaaaccacgactataccctttggctccccgaagatccctctctcagcctcatatgtgacatctttacttcctccaacggccaaaaagcaatgaacggatcacggatatgtggcttcaaagatgaacggggattttacagaagtttgaaaggggcgtgcaaactgactttgtgtggcaaacccggcatccgcttgttcgatgggacgtgggtcagcttcgccaggccggacgtgcatgtgtggtgcacccccaaccagctggtcaatatacacaatgaccgccttgatgagatagaacacttgattgtagaggacattattaagcgcagggaggagtgccttgatacgcttgaaacaatcctgatgagccagagcataagttttaggcggctctcccattttaggaaactcgtaccgggttatggtaaagcttacaccgtcctgaacggcagtttgatggagaccaacgtctattacaagagggtagataagtggaccgatattttgccgtctaaaggttgtctcaaggttgggcaacaatgcatggatcctgttaaaggtgtgttctttaacgggataattaagggtcccgacggacagattcttattcctgaaatgcaaagtgagcaacttaaacagcacatggatctcttgaaagccgctgttttcccattgcgacaccctctgattgacagaggcgctgtctttaagaaggatggtgacgcggacgatttcgttgacctccatatgccagacgtacacaaaagtgttagtgatgttgatctcggtttgcctcattggggtctttggctgatgataggtgccgctgtcgttgcatttatggtactcatctgtcttctcagagtttgttgcaagcgggtgggccgcaggcggagcccccacgcaactcaagaaataccactgagcgtttccagtgccccggtaccacgagcaaaagttgtctctagctgggagtcatacaaagggctccccggtacttga

SEQ ID NO: 3 - protein, 522 aa, Artificial Sequence
  MOKV glycoprotein Protein
MNLPCLTVILILFTKYSLGEFPLYTIPEKIEKWTPIDMIHLSCPNNLLSQEEGCNAETPFTYFELKSGYLAHQKVQGFTCTGVVNEAETYTNFVGYVTTTFKRKHFRPTVAACRDAYNWKVSGDPRYEESLHTPYPDSSWLRTVTTTKESLLIISPSIVEMDIYGRTLHSPMFPSGTCSKLYPSVPSCKTNHDYTLWLPEDPSLSLICDIFTSSNGQKAMNGSRICGFKDERGFYRSLKGACKLTLCGKPGIRLFDGTWVSFARPDVHVWCTPNQLVNIHNDRLDEIEHLIVEDIIKRREECLDTLETILMSQSISFRRLSHFRKLVPGYGKAYTVLNGSLMETNVYYKRVDKWTDILPSKGCLKVGQQCMDPVKGVFFNGIIKGPDGQILIPEMQSEQLKQHMDLLKAAVFPLRHPLIDRGAVFKKDGDADDFVDLHMPDVHKSVSDVDLGLPHWGLWLMIGAAVVAFMVLICLLRVCCKRVGRRRSPHATQEIPLSVSSAPVPRAKVVSSWESYKGLPGT

SEQ ID NO: 4 - DNA, 1575 bp, Artificial Sequence
  chimeric MOKV/RABV glycoprotein DNA
atggtccctcaggctctgctgttcgtcccactgctggtcttccctctgtgctttggcaagttccctatctacactattcccgacaagctgggcccctggtctcctatcgatattcaccatctgagttgccctaacaatctggtggtcgaggaggagggctgcaacgccgagacacctttcacgtactttgagctcaaatcaggctatttggcgcatcagaaggttcaggggtttacctgcactggagtagtgaatgaagcggaaacctataccaactttgtaggatatgttacaacaacgtttaagcggaaacatttcagacccactgtagcagcatgcagggacgcatataattggaaggtgagcggtgaccctcggtatgaggaaagtcttcacaccccctaccctgatagttcctggttgcggaccgtcaccacaaccaaagagagtctgctgataataagtccgtccattgtcgagatggatatttatggccggactcttcacagcccgatgttcccgtcagggacgtgtagcaagctgtacccgtcagtaccatcctgcaagacaaaccacgactataccctttggctccccgaagatccctctctcagcctcatatgtgacatctttacttcctccaacggccaaaaagcaatgaacggatcacggatatgtggcttcaaagatgaacggggattttacagaagtttgaaaggggcgtgcaaactgactttgtgtggcaaacccggcatccgcttgttcgatgggacgtgggtcagcttcgccaggccggacgtgcatgtgtggtgcacccccaaccagctggtcaatctgcacgacttcaggagcgacgagatcgaacatctggtggtcgaggaactggtgcgaaaaagggaggaatgtctggatgccctggagtccatcatgactaccaagagcgtgagcttcaggaggctgtctcacctgcgaaagctggtgcccggcttcggcaaagcctacaccatctttaacaagacactgatggaagcagacgcccattataaatcagtggagacctggaatgaaattctgccaagcaagggctgcctgcgggtgggcggacgctgtcacccacatgtgaacggcgtcttctttaatggaatcattctggggcccgacggcaacgtgctgatccctgagatgcagtctagtctgctgcagcagcacatggagctgctggaatcaagcgtgattcctctggtccatccactggcagatccctccacagtgttcaaagacggagatgaggccgaagactttgtggaagtccacctgcctgatgtgcataaccaggtgtctggcgtcgacctgggactgccaaattggggcaagtacgtgctgctgagtgctggagcactgactgccctgatgctgatcattttcctgatgacctgctgtcggcgcgtgaacagaagtgagcccactcagcacaatctgcgaggaaccgggagagaagtgtcagtcacacctcagagcgggaaaatcattagtagttgggaatcacataaaagcgggggcgagaccaggctgtga

SEQ ID NO: 5 - protein, 524 aa, Artificial Sequence
  chimeric MOKV/RABV glycoprotein Protein
MVPQALLFVPLLVFPLCFGKFPIYTIPDKLGPWSPIDIHHLSCPNNLVVEEEGCNAETPFTYFELKSGYLAHQKVQGFTCTGVVNEAETYTNFVGYVTTTFKRKHFRPTVAACRDAYNWKVSGDPRYEESLHTPYPDSSWLRTVTTTKESLLIISPSIVEMDIYGRTLHSPMFPSGTCSKLYPSVPSCKTNHDYTLWLPEDPSLSLICDIFTSSNGQKAMNGSRICGFKDERGFYRSLKGACKLTLCGKPGIRLFDGTWVSFARPDVHVWCTPNQLVNLHDFRSDEIEHLVVEELVRKREECLDALESIMTTKSVSFRRLSHLRKLVPGFGKAYTIFNKTLMEADAHYKSVETWNEILPSKGCLRVGGRCHPHVNGVFFNGIILGPDGNVLIPEMQSSLLQQHMELLESSVIPLVHPLADPSTVFKDGDEAEDFVEVHLPDVHNQVSGVDLGLPNWGKYVLLSAGALTALMLIIFLMTCCRRVNRSEPTQHNLRGTGREVSVTPQSGKIISSWESHKSGGETRL

(19) United States
(12) Patent Application Publication
(10) Pub. No.: US 2023/0398201 A1
(43) Pub. Date: Dec. 14, 2023

(54) GENE SHUFFLED LYSSAVIRUS VACCINE

(71) Applicant: Thomas Jefferson University, philadelphia, PA (US)

(72) Inventors: Matthias Johannes Schnell, Harleysville, PA (US); Christoph Wirblich, Philadelphia, PA (US)

(21) Appl. No.: 18/034,720

(22) PCT Filed: Nov. 2, 2021
(86) PCT No.: PCT/US21/57743

§ 371 Date: May 1, 2023

Related U.S. Application Data
(60) Provisional application No. 63/108,936, filed on Nov. 3, 2020.

(51) Int. Cl. A61K 39/12; A61P 31/14; C12N 7/00

(52) U.S. Cl. CPC A61K 39/12; A61P 31/14; C12N 7/00; C12N 2760/20134; C12N 2760/20143; C12N 2760/20122; C12N 2760/20171; A61K 2039/5252

(57) ABSTRACT

The present invention includes a vaccine comprising a nucleic acid comprising (a) a nucleotide sequence encoding a rabies virus nucleoprotein (N) or a portion thereof and (b) a nucleotide sequence encoding a glycoprotein (G) (e.g., a RABV glycoprotein, a MOKV glycoprotein, or a chimeric MOKV/RABY glycoprotein), or a portion thereof positioned immediately 3′ to the nucleoprotein (N) gene sequence.

### CROSS-REFERENCE TO RELATED APPLICATION

The present application is entitled to priority under 35 U.S.C. § 119(e) to U.S. Provisional Patent Application No. 63/108,936 filed Nov. 3, 2020, which is hereby incorporated by reference in its entirety herein.

### STATEMENT REGARDING FEDERALLY SPONSORED RESEARCH

This invention was made with government support under 5R21AI128175-02 awarded by the National Institutes of Health. The government has certain rights in the invention.

### SEQUENCE LISTING

The ASCII text file named “205961_7066WO1SequenceListing” created on Oct. 28, 2021, comprising 28 Kbytes, is hereby incorporated by reference in its entirety.

### BACKGROUND OF THE INVENTION

Emerging infectious diseases are on the rise. The recent outbreaks of Ebola virus, Zika virus, and SARS-CoV-2 highlight our lack of preparedness: despite their relation to known diseases, few therapeutics were available for rapid distribution. Zoonotic diseases are particularly threatening, because they often move to new hosts opportunistically and can rarely be eradicated on a global scale.

Rabies is a neglected infectious disease that is responsible for an estimated 59,000 global human deaths annually, roughly the same number of deaths caused annually by influenza in the United States. Whereas millions of people survive influenza each year, fewer than 30 cases of human rabies survival have been documented. The number of human rabies deaths is likely underestimated, as studies in developing countries with poor health infrastructure suggest.

Rabies virus (RABV)-induced encephalitis is the most lethal viral infection known to humankind when no intervention is applied prior to symptoms. Less known is that RABV-related lyssaviruses cause the same zoonotic disease, have similar mortality rates as RABV, but are far less studied (Banyard et al., 2014; Evans et al., 2012). The lyssavirus genus is comprised of 17 single-stranded, negative-sense RNA viruses divided into at least three phylogroups (RABV being categorized in phylogroup I) (Markotter and Coertse, 2018). Classical RABV circulates on all continents but Antarctica; non-RABV lyssaviruses are endemic in Europe, Africa, Asia, and Australia (Fisher et al., 2018).

A need exists for more effective lyssavirus vaccines and more efficient production of lyssavirus vaccines. The present invention addresses and satisfies this need.

### SUMMARY OF THE INVENTION

In some aspects, an isolated nucleic acid is provided encoding a recombinant lyssavirus comprising a nucleotide sequence encoding at least a portion of the genome of a rabies virus, wherein the at least a portion of the genome of the rabies virus comprises (a) a nucleotide sequence encoding a rabies virus nucleoprotein (N) or a portion thereof and wherein the recombinant lyssavirus further comprises (b) a nucleotide sequence encoding a glycoprotein (G) or a portion thereof positioned immediately 3′ to the nucleotide sequence encoding the nucleoprotein (N).

In some embodiments, the glycoprotein (G) is selected from a RABV glycoprotein, a MOKV glycoprotein, and a chimeric MOKV/RABV glycoprotein.

In some embodiments, the recombinant lyssavirus is a SADB-19 rabies virus strain.

In some embodiments, the nucleic acid encoding the RABV glycoprotein, the chimeric MOKV/RABV glycoprotein, or a portion thereof comprises a mutation that results in insertion of glutamic acid in place of arginine at position 333 of the RABV glycoprotein.

In some embodiments the nucleotide sequence encoding the chimeric MOKV/RABV glycoprotein or a portion thereof comprises a nucleotide sequence encoding a RABV clip domain, a nucleotide sequence encoding a MOKV core domain, and a nucleotide sequence encoding a RABV flap domain.

In some embodiments, the nucleotide sequence encoding the chimeric MOKV/RABV glycoprotein or a portion thereof comprises a nucleotide sequence encoding a MOKV clip domain, a nucleotide sequence encoding a RABV core domain, and a nucleotide sequence encoding a MOKV flap domain.

In some embodiments, the nucleotide sequence (b) encoding the glycoprotein (G) is positioned immediately 5′ to (c) a nucleotide sequence encoding a rabies virus phosphoprotein (P).

In some embodiments, the nucleic acid comprises a nucleotide sequence having at least 85% sequence identity to SEQ ID NO: 1, at least 85% sequence identity to SEQ ID NO: 2, or at least 85% sequence identity to SEQ ID NO: 4.

In some embodiments, the nucleic acid comprises a nucleotide sequence having at least 90% sequence identity to SEQ ID NO: 1, at least 90% sequence identity to SEQ ID NO: 2, or at least 90% sequence identity to SEQ ID NO: 4.

In some embodiments, the nucleic acid comprises a nucleotide sequence having at least 95% sequence identity to SEQ ID NO: 1, at least 95% sequence identity to SEQ ID NO: 2, or at least 95% sequence identity to SEQ ID NO: 4.

In some embodiments, the nucleic acid comprises a nucleotide sequence having at least 99% sequence identity to SEQ ID NO: 1, at least 99% sequence identity to SEQ ID NO: 2, or at least 99% sequence identity to SEQ ID NO: 4.

In some embodiments, the nucleic acid comprises the nucleotide sequence set forth in SEQ ID NO: 1, SEQ ID NO:2, or SEQ ID NO: 4.

In some embodiments, the nucleic acid encodes a recombinant rabies virus.

In some aspects, the invention provides an isolated nucleic acid comprising (a) a nucleotide sequence encoding a rabies virus nucleoprotein (N) or a portion thereof and (b) a nucleotide sequence encoding a glycoprotein (G) or a portion thereof positioned immediately 3′ to the nucleotide sequence encoding the nucleoprotein (N).

In some embodiments, the nucleotide sequence encoding the RABV glycoprotein, the MOKV glycoprotein, the chimeric MOKV/RABV glycoprotein, or portion thereof is positioned immediately 5′ to (c) a sequence nucleotide encoding a rabies virus phosphoprotein (P).

In some embodiments, the nucleotide sequence encoding the rabies virus phosphoprotein (P) is positioned immediately 5′ to (d) a nucleotide sequence encoding a rabies virus protein (M) and wherein the nucleotide sequence encoding protein (M) is positioned immediately 5′ to (e) a nucleotide sequence encoding a rabies virus protein (L).

In some embodiments, the nucleic acid comprises a nucleotide sequence having at least 85% sequence identity to SEQ ID NO: 1, at least 85% sequence identity to SEQ ID NO: 2, or at least 85% sequence identity to SEQ ID NO: 4.

In some embodiments, the isolated nucleic acid comprises a nucleotide sequence having at least 90% sequence identity to SEQ ID NO: 1, at least 90% sequence identity to SEQ ID NO: 2, or at least 90% sequence identity to SEQ ID NO: 4.

In some embodiments, the nucleic acid comprises a nucleotide sequence having at least 95% sequence identity to SEQ ID NO: 1, at least 95% sequence identity to SEQ ID NO: 2, or at least 95% sequence identity to SEQ ID NO: 4.

In some embodiments, the nucleic acid comprises a nucleotide sequence having at least 99% sequence identity to SEQ ID NO: 1, at least 99% sequence identity to SEQ ID NO: 2, or at least 99% sequence identity to SEQ ID NO: 4.

In some embodiments, the nucleic acid comprises the nucleotide sequence set forth in SEQ ID NO: 1, SEQ ID NO: 2, or SEQ ID NO: 4.

In some embodiments, the nucleic acid encoding the recombinant virus is codon optimized for expression in a host cell.

In some embodiments, the host cell is a mammalian cell.

In some aspects, the invention provides a recombinant virus encoded by a nucleic acid sequence comprising at least a portion of the genome of the rabies virus, wherein the at least a portion of the genome of the rabies virus comprises (a) a nucleotide sequence encoding a rabies virus nucleoprotein (N) or a portion thereof and wherein the nucleic acid sequence further comprises (b) a nucleotide sequence encoding a glycoprotein (G) or a portion thereof positioned immediately 3′ to the nucleotide sequence encoding the nucleoprotein (N).

In some embodiments, the glycoprotein (G) encoded by the recombinant virus is selected from a RABV glycoprotein, a MOKV glycoprotein, and a chimeric MOKV/RABV glycoprotein.

In some embodiments, the nucleotide sequence encoding the RABV glycoprotein, the chimeric MOKV/RABV glycoprotein, or portion thereof comprises a mutation that results in insertion of glutamic acid in place of arginine at position 333 of the RABV glycoprotein.

In some embodiments, the nucleotide sequence encoding the chimeric MOKV/RABV glycoprotein or portion thereof comprises (a) nucleotide sequence encoding a RABV clip domain, (b) a nucleotide sequence encoding a MOKV core domain and (c) a nucleotide sequence encoding a RABV flap domain.

In some embodiments, the nucleotide sequence encoding the chimeric MOKV/RABV glycoprotein or a portion thereof comprises (a) nucleotide sequence encoding a MOKV clip domain, (b) a nucleotide sequence encoding a RABV core domain, and (c) a nucleotide sequence encoding a MOKV flap domain.

In some embodiments, the recombinant virus is a recombinant rabies virus.

In some embodiments, a recombinant virus is encoded by a nucleic acid recited in the specification.

In some embodiments, a vector comprises the nucleic acid of any one of the nucleic acid sequences recited in the specification.

In some embodiments, a vaccine comprises the recombinant virus encoded by the isolated nucleic acid as recited in the specification, and a pharmaceutically acceptable carrier.

In some embodiments, the vaccine further comprises an adjuvant.

In some embodiments the vaccine comprises a virus that is deactivated.

In some aspects, a method is provided for generating an immune response against a lyssavirus in a subject in need thereof, the method comprising administering to the subject an effective amount of the recombinant virus of recited in the specification, a recombinant virus encoded by the isolated nucleic acid recited in the specification, or the vaccine recited in the specification.

In some aspects, a method is provided for vaccinating a subject against a lyssavirus, the method comprising administering to the subject an effective amount of the recombinant virus recited in the specification, a recombinant virus encoded by the isolated nucleic acid recited in the specification, or the vaccine recited in the specification.

In some aspects, a method is provided for providing immunity against a lyssavirus in a subject, the method comprising administering to the subject an effective amount of the recombinant virus recited in the specification, a recombinant virus encoded by the isolated nucleic acid recited in the specification, or the vaccine recited in the specification.

In some aspects, a method is provided for treating and/or preventing a disease or disorder associated with a lyssavirus in a subject in need thereof, the method comprising administering to the subject an effective amount of the recombinant virus recited in the specification, a recombinant virus encoded by the isolated nucleic acid recited in the specification, or the vaccine recited in the specification.

In some aspects, a method is provided for increasing immunogenicity against a lyssavirus in a subject in need thereof, the method comprising administering to the subject an effective amount of the recombinant virus recited in the specification, a recombinant virus encoded by the isolated nucleic acid recited in the specification, or the vaccine recited in the specification.

In some embodiments, the subject is a mammal.

In some embodiments, the lyssavirus is a rabies virus.

In some embodiments, a method is provided for increasing expression of a recombinant lyssavirus in a host cell, the method comprising expressing in the host cell a nucleic acid sequence recited in the specification.

In some embodiments, the recombinant lyssavirus is a recombinant rabies virus.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1: Map of BNSP333-CoG333-AG (a gene shuffled attenuated rabies vaccine expressing rabies virus glycoprotein optimized for codon use in mammalian animals). The nucleotide sequence is provided by SEQ ID NO: 1.

FIGS. 2A-2C: Construction and recovery of a chimeric lyssavirus G vaccine. FIG. 2A depicts viral genome schematics. BNSP333 is the parent vaccine vector based on RABV strain SAD B19. Its G is located in the native fourth position and contains the attenuating R333E mutation. BNSPDG is based on BNSP333 but lacks the native G. All of the following experimental constructs are based on BNSPDG: rRABV contains a human codon-optimized (c.o.) RABV G with the attenuating mutation R333E at the second position; rMOKV contains human c.o. MOKV G at the second position; rChimeral (LyssaVax) contains Chimeric G 1, with the attenuating R333E mutation, at the second position; and rChimera2 contains Chimeric G 2 at the second position. FIG. 2B depicts infection immunofluorescence. VERO cells infected with either LyssaVax (left column), rMOKV (second column), rRABV (third column), or uninfected (right column) were fixed and stained with a DyLight 488-conjugated human anti-RABV G mAb 4C12 and mouse anti-MOKV G sera. Nuclei were labeled in blue by DAPI. Scale bars represent 50 μm. FIG. 2C depicts an analysis of purified virions. 3-μg viral particles denatured and resolved by SDS-PAGE, then total protein stained with SYPRO Ruby. Viral proteins are indicated.

FIGS. 3A-3C: Glycoprotein expression comparison in a dual G construct. FIG. 3A depicts viral genome schematics. BNSP333 (top) is the parent vaccine vector based on RABV strain SAD B19. Its G is located in the native 4th position and contains the attenuating R333E mutation. BNSP333-RABVG contains an additional human codon-optimized RABV G at the 2nd position (also contains the attenuating R333E mutation); BNSP333-MOKVG contains an additional human codon-optimized MOKV G at the 2nd position. All RABV Gs in the above constructs have the attenuating R333E mutation. FIG. 3B depicts infection immunofluorescence. VERO cells infected with either BNSP333-MOKV-G (left column), rMOKV (second column), BNSP333-RABV-G (third column), rRABV (fourth column), or uninfected (right column) were fixed and stained with a DyLight 488-conjugated human anti-RABV G mAb 4C12 (green) and mouse anti-MOKV G sera (red). Nuclei were labeled in blue by DAPI. Scale bars represent 100 μm. FIG. 3C is a multi-step growth curve. BSR cells were infected at MOI 0.01.

FIGS. 4A-4B: Live virus pathogenicity profiles. Male (M, dashed line) and female (F, solid line) mice (n=5 per sex, per group) were inoculated with 10⁵ FFU of live virus and monitored for 28 days. FIG. 4A depicts survival after intranasal infection. Survival was analyzed using the log-rank Mantel-Cox test: **, p=0.0011; ****p<0.0001. Statistical differences between male and female mice in FIG. 4A: SPBN, **, p=0.0027; MOKV, *, p=0.0290. FIG. 4B depicts survival after intramuscular infection. Survival was analyzed using the log-rank Mantel-Cox test: **, p=0.0015. Statistical differences between male and female mice in FIG. 4B: CVS-N2c, ns.

FIGS. 5A-5C: Humoral response to LyssaVax. FIG. 5A is a schematic timeline of immunization (syringe), sera collection (drop), and challenge (bolt) through necropsy (NEC). FIG. 5B depicts the development of antibodies over time in groups of mice (n=10 mice per group, analyzed in triplicate, mean±SD) immunized three times with either LyssaVax, rRABV, or rMOKV. Graphs compare half-maximal responses (EC₅₀ₛ) between sera from immune mice probed against RABV G antigens in ELISA format. Day 0 samples did not seroconvert, so EC₅₀ values were not calculated. FIG. 5C depicts the development of antibodies over time in groups of mice (n=10 mice per group, analyzed in triplicate, mean±SD) immunized three times with either LyssaVax, rRABV, or rMOKV. Graphs compare half-maximal responses (EC₅₀s) between sera from immune mice probed against MOKV G antigens in ELISA format. Day 0 samples did not seroconvert, so EC₅₀ values were not calculated. Analysis within time points of FIGS. 4B and 4C by Kruskal-Wallis test and Dunn's multiple comparisons test (*p<0.05, **p<0.01, ***p<0.001, ****p<0.0001 for adjusted p values; n.s. is not significant). See also FIGS. 14A-14E.

FIG. 6: RABV neutralizing titers over time. Development of RABV neutralizing antibodies over time, averaged from mice in groups of mice (n=10 mice per group, analyzed in duplicate, mean±SD) immunized on days 0, 7, and 28 with either rRABV, rMOKV, or LyssaVax, or mock immunized. See FIG. 5A for full immunization scheme. Titers were calculated in international units (IU) per milliliter by comparison with the US standard rabies immune globulin. Level of detection (LOD) was 4 IU/mL. Two-way ANOVA and Tukey's multiple comparisons tests were performed. *p=0.034, comparing rRABV and LyssaVax. See also Table 1 and FIG. 15.

FIGS. 7A-7E: MOKV G pseudotype neutralizing titers. VNA titers against MOKV G pseudotype viruses (PTVs). PTVs made by trans-complementing VSV-DG-NanoLuc-EGFP with MOKV G (FIG. 10). VNA titers measured in sera from mice immunized with either LyssaVax (open circle), rRABV (open square), or rMOKV (open triangle), or mock immunized with PBS. FIG. 7A depicts average titers shown over time on day 7. FIG. 7B depicts average titers shown over time on day 14. FIG. 7C depicts average titers shown over time on day 35. (FIGS. 7A-7C: n=10 mice per group, analyzed in triplicate, mean±SD). FIG. 7D depicts pseudotype neutralization by the mAb 1409-7. Luminescence data background subtracted using paired sera from day 0 and normalized to 100% infection in no-sera controls. FIG. 7E depicts serum IC₅₀ data analyzed by the Mann-Whitney test (*p=0.0133).

FIG. 8: Survival after challenge with pathogenic RABV and rMOKV. Overall survival data post-challenge (p.c.) with either live RABV (SPBN) or live rMOKV (n=5 mice per immunization group per challenge virus). Survival was analyzed using the log rank Mantel-Cox test. See FIGS. 16A-16H for weight loss curves.

FIGS. 9A-9H: Microneutralization assay with panel of WT lyssaviruses from Phylogroup I (FIG. 9A-9D) and Phylogroup II (FIG. 9E-9H). FIG. 9A,RABV(CVS-11); FIG. 9B, WT EBLV1; FIG. 9C, WT DUVV; FIG. 9D, WT TRKV; FIG. 9E, WT MOKV; FIG. 9F, WT LBV(B); FIG. 9G, WT LBV(D); FIG. 9H, WT SHIBV. Neutralizing antibody 50% endpoint titers in sera from mice 47 days after first immunization with rRABV, rRABV with the adjuvant GLA-SE, LyssaVax, or LyssaVax with GLA-SE (n=10 mice per group, analyzed in duplicate, mean±SD). See FIG. 5A for immunization scheme. All titers normalized to day 0 sera were pooled within groups. Upper limit endpoint: 2,795. Microneutralizations were analyzed using an ordinary one-way ANOVA test with Tukey's multiple comparisons (*p<0.05, **p<0.01, ***p<0.001, ****p<0.0001 for adjusted p values; n.s. is not significant).

FIG. 10: Design of single-round VSV pseudotyped with MOKV G. Related to FIGS. 7A-7E. MOKV G pseudotype viruses (PTVs) are single-round infectious virons which induce NanoLuciferase (NanoLuc) and EGFP expression in cells upon infection.

FIGS. 11A-11F: Structure-based design of chimeric lyssavirus glycoproteins. FIG. 11A depicts a representative structural model of a lyssavirus glycoprotein (G) with proposed structural domains highlighted. FIG. 11B depicts a structural model of the Chimeric G 1 clip domain highlighted in white, and core and flap domains highlighted in blue or red, corresponding to patterns in FIG. 11E and FIG. 11F. FIG. 11C depicts a structural model of the Chimeric G 2 clip domain, and core and flap domains, corresponding to patterns in FIG. 11E and FIG. 11F. FIG. 11D is a linear schematic of a representative lyssavirus G monomer wherein the proposed structural domains of the ectodomain are noted, as are the antigenic regions as they are known to exist on the RABV G: site I (residues 224-229), site II (34-42 and 198-200), site III (330-338), site IV (263-264), and minor site “a” (342-343). TM, transmembrane domain. FIG. 11E is a linear schematic of the RABV G/MOKV G chimeric G named Chimeric G 1, wherein R333E, attenuating mutation at RABV G residue 333. FIG. 11F is a linear schematic of the RABV G/MOKV G chimeric G named Chimeric G 2. See also FIGS. 12A and 12B and FIG. 13.

FIGS. 12A-12B: Comparison between model and crystal structures of RABV G. Related to FIGS. 11A-11F. FIG. 12A is an overlay of structural model of RABV G generated using Phyre2 and crystal structure of RABV G (Yang et al., 2020). FIG. 12B is a crystal structure of RABV G (Yang et al., 2020) colored to highlight the clip, core, and flap domains.

FIG. 13: Immunofluorescence of transfected chimeric lyssavirus glycoproteins. Related to FIGS. 11A-11F. VERO cells transfected with pCAGGS expression plasmids containing the genes of either Chimeric G 1 (left column), Chimeric G 2 (second column), MOKV G (third column), or RABV G (fourth column). Two days post-transfection, cells were fixed with 4% paraformaldehyde and stained with a DyLight 488-conjugated human anti-RABV G mAb 4C12 (top row), mouse anti-MOKV G sera (middle row) or mouse anti-RABV G sera (bottom row). Sera specific to RABV G and MOKV G both bind to Chimeric Gs 1 and 2, whereas they are not otherwise cross-reactive with the other glycoprotein. 4C12 binds only to Chimeric G 1. Brightness adjusted with sets transfected with the same glycoprotein and stained with the same antibody. Scale bars represent 100 μm.

FIGS. 14A-14K: Humoral response to recombinant LyssaVax (full dilution curves). Related to FIGS. 3A-3C. Sera from mice immunized with rRABV, rMOKV, LyssaVax (purple), or PBS (mock, dark gray) at different days post-immunization were assayed by ELISA against RABV G (FIGS. 14A-14E) or MOKV G (FIGS. 14F-14J) antigens (n=10 per group, mean±SD). Control antibodies (black): 1C5=mouse anti-RABV G mAb; 1409-7=mouse anti-MOKV G mAb; 2°=goat anti-mouse IgG (H+L). FIG. 14A depicts sera at 0 days post-immunization. FIG. 14B depicts sera at 7 days post-immunization. FIG. 14C depicts sera at 14 days post-immunization. FIG. 14D depicts sera at 35 days post-immunization. FIG. 14E depicts sera at 58 days post-immunization. FIG. 14F depicts sera at 0 days post-immunization. FIG. 14G depicts sera at 7 days post-immunization. FIG. 14H depicts sera at 14 days post-immunization. FIG. 14I depicts sera at 35 days post-immunization. FIG. 14J depicts sera at 58 days post-immunization. FIG. 14K depicts sera from mice immunized with controls (2° and 1409-7).

FIG. 15: Lower threshold of RABV neutralizing titers in sera from rMOKV-immune mice. Related to FIG. 6. RABV neutralizing antibody titers in individual mice (n=10 per group) at days 28 and 56 after immunization with rMOKV. See FIG. 5A for full immunization scheme. Solid symbols indicate mice which were later challenged with rMOKV (4-6 to 4-10). Symbols with blue interiors indicate mice which were later challenged with SPBN. Titers were calculated in international units (IU) per ml by comparison to the U.S. standard rabies immune globulin. Level of detection (LOD) was 0.2 IU/ml (dotted line). Dashed line at 0.5 IU/ml indicates accepted level of RABV-neutralizing antibodies necessary for protection. Exact titers listed in Table 1.

FIGS. 16A-16H: Lower threshold of RABV neutralizing titers in sera from rMOKV-immune mice. Related to FIG. 8. FIGS. 16A-16H depict weight curves of mice immunized with a vaccine that were challenged i.n. with either 10⁵ FFU of live RABV (SPBN strain, FIGS. 16A-16D) or rMOKV (FIGS. 16E-16H) at day 58 post-immunization (p.i.). Mice which exhibited symptoms of disease or lost greater than 25% of day 0 weight were euthanized. FIG. 16A depicts weight curves of mice immunized with a mock vaccine. FIG. 16B depicts weight curves of mice immunized with LyssaVax. FIG. 16C depicts weight curves of mice immunized with rRABV. FIG. 16D depicts weight curves of mice immunized with rMOKV. FIG. 16E depicts weight curves of mice immunized with a mock vaccine. FIG. 16F depicts weight curves of mice immunized with LyssaVax. FIG. 16G depicts weight curves of mice immunized with rRABV. FIG. 16H depicts weight curves of mice immunized with rMOKV.

### DETAILED DESCRIPTION

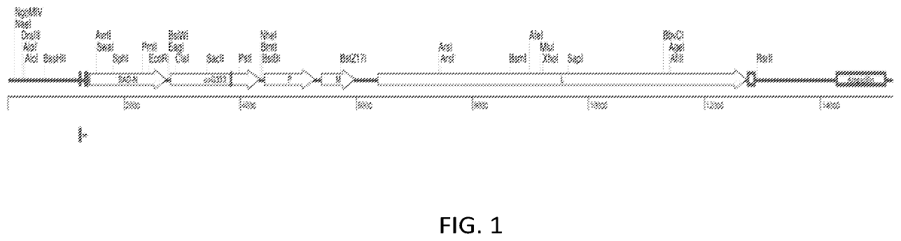

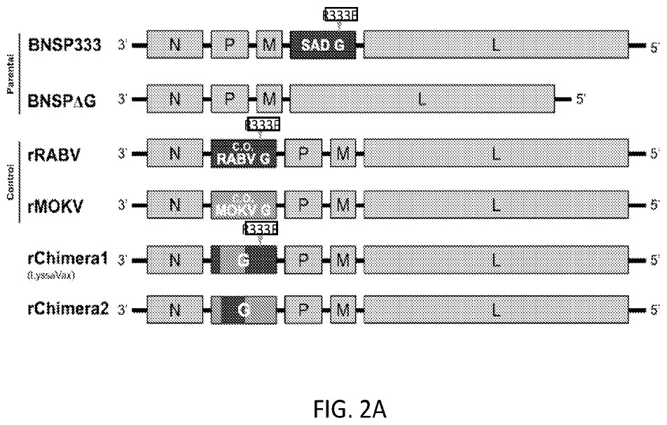

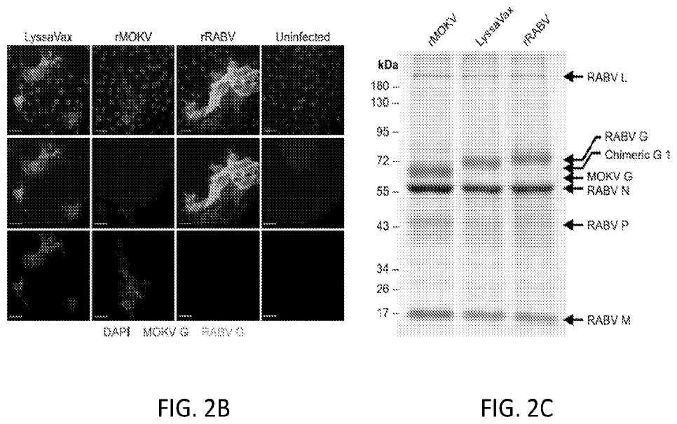

The present disclosure relates to a lyssavirus vaccine comprising a recombinant virus, where the recombinant virus is encoded by a nucleic acid comprising a sequence encoding at least a portion of a rabies virus genome, wherein the sequence encoding the at least a portion of a rabies virus genome comprises (a) a sequence encoding a nucleoprotein (N) and (b) a sequence encoding an RABV glycoprotein or portion thereof, an MOKV glycoprotein or portion thereof, or a chimeric MOKV/RABV glycoprotein or portion thereof, positioned closer to the 3′ end of the rabies genome (after N) (FIG. 1 and FIG. 2A), which results in higher expression levels. In some embodiments, the gene has been optimized for codon usage of mammalian cells (human) to increase the expression level further. In addition, in certain embodiments, the glycoprotein (G) contains the so-called 333 mutation in the RABV G protein (Arg to Glu), which significantly reduces neurotropism of RABV. This vaccine is expected to be highly attenuated with increased immunogenicity in the immunized host.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

The term “antibody” or “Ab” as used herein, refers to a protein, or polypeptide sequence derived from an immunoglobulin molecule, which specifically binds to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv) and humanized antibodies (Harlow et al., 1998, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426). An antibody may be derived from natural sources or from recombinant sources. Antibodies are typically tetramers of immunoglobulin molecules.

The term “ameliorating” or “treating” means that the clinical signs and/or the symptoms associated with a disease are lessened as a result of the actions performed. The signs or symptoms to be monitored will be well known to the skilled clinician.

As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The term “biological” or “biological sample” refers to a sample obtained from an organism or from components (e.g., cells) of an organism. The sample may be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

As used herein, the terms “control,” or “reference” are used interchangeably and refer to a value that is used as a standard of comparison.

The term “immunogenicity” as used herein, refers to the innate ability of an antigen or organism to elicit an immune response in an animal when the antigen or organism is administered to the animal. Thus, “enhancing the immunogenicity” refers to increasing the ability of an antigen or organism to elicit an immune response in an animal when the antigen or organism is administered to an animal. The increased ability of an antigen or organism to elicit an immune response can be measured by, among other things, a greater number of antibodies that bind to an antigen or organism, a greater diversity of antibodies to an antigen or organism, a greater number of T-cells specific for an antigen or organism, a greater cytotoxic or helper T-cell response to an antigen or organism, a greater expression of cytokines in response to an antigen, and the like.

As used herein, the terms “eliciting an immune response” or “immunizing” refer to the process of generating a B cell and/or a T cell response against a heterologous protein.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Heterologous antigens” used herein to refer to an antigen that is not endogenous to the organism comprising or expressing an antigen. As an example, a virus vaccine vector comprising or expressing a viral or tumor antigen comprises a heterologous antigen. The term “Heterologous protein” as used herein refers to a protein that elicits a beneficial immune response in a subject (i.e. mammal), irrespective of its source.

The term “specifically binds”, “selectively binds” or “binding specificity” refers to the ability of the humanized antibodies or binding compounds of the invention to bind to a target epitope with a greater affinity than that which results when bound to a non-target epitope. In certain embodiments, specific binding refers to binding to a target with an affinity that is at least 10, 50, 100, 250, 500, or 1000 times greater than the affinity for a non-target epitope.

As used herein, by “combination therapy” is meant that a first agent is administered in conjunction with another agent. “In combination with” or “In conjunction with” refers to administration of one treatment modality in addition to another treatment modality. As such, “in combination with” refers to administration of one treatment modality before, during, or after delivery of the other treatment modality to the individual. Such combinations are considered to be part of a single treatment regimen or regime.

“Humoral immunity” or “humoral immune response” both refer to B-cell mediated immunity and are mediated by highly specific antibodies, produced and secreted by B-lymphocytes (B-cells).

“Prevention” refers to the use of a pharmaceutical compositions for the vaccination against a disorder.

“Adjuvant” refers to a substance that is capable of potentiating the immunogenicity of an antigen. Adjuvants can be one substance or a mixture of substances and function by acting directly on the immune system or by providing a slow release of an antigen. Examples of adjuvants are aluminium salts, polyanions, bacterial glycopeptides and slow release agents as Freund's incomplete.

“Delivery vehicle” refers to a composition that helps to target the antigen to specific cells and to facilitate the effective recognition of an antigen by the immune system. The best-known delivery vehicles are liposomes, virosomes, microparticles including microspheres and nanospheres, polymers, bacterial ghosts, bacterial polysaccharides, attenuated bacteria, virus like particles, attenuated viruses and ISCOMS.

The term “expression” as used herein is defined as the transcription and/or translation of a particular nucleotide sequence driven by its promoter.

As used herein, the term “expression cassette” means a nucleic acid sequence capable of directing the transcription and/or translation of a heterologous coding sequence. In some embodiments, the expression cassette comprises a promoter sequence operably linked to a sequence encoding a heterologous protein. In some embodiments, the expression cassette further comprises at least one regulatory sequence operably linked to the sequence encoding the heterologous protein.

“Incorporated into” or “encapsulated in” refers to an antigenic peptide that is within a delivery vehicle, such as microparticles, bacterial ghosts, attenuated bacteria, virus like particles, attenuated viruses, ISCOMs, liposomes and preferably virosomes.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “fusion protein” as used herein refers to a protein wherein the protein comprises two or more proteins linked together by peptide bonds or other chemical bonds. The proteins can be linked together directly by a peptide or other chemical bond, or with one or more amino acids between the two or more proteins, referred to herein as a spacer.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “RNA” as used herein is defined as ribonucleic acid.

“Transform”, “transforming”, and “transformation” is used herein to refer to a process of introducing an isolated nucleic acid into the interior of an organism.

The term “treatment” as used within the context of the present invention is meant to include therapeutic treatment as well as prophylactic, or suppressive measures for the disease or disorder. As used herein, the term “treatment” and associated terms such as “treat” and “treating” means the reduction of the progression, severity and/or duration of a disease condition or at least one symptom thereof. The term ‘treatment’ therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

The term “equivalent,” when used in reference to nucleotide sequences, is understood to refer to nucleotide sequences encoding functionally equivalent polypeptides. Equivalent nucleotide sequences will include sequences that differ by one or more nucleotide substitutions, additions- or deletions, such as allelic variants; and will, therefore, include sequences that differ from the nucleotide sequence of the nucleic acids described herein due to the degeneracy of the genetic code.

As used herein, a sequence that is positioned “immediately 3′” to another sequence means that the sequence is positioned 3′ to (i.e. downstream of) the other sequence without a protein coding sequence in between the two sequences. A non-coding sequence can be between the two sequences. For example, if a G gene is “immediately 3′” to an N gene, the G gene is positioned 3′ to the N gene, without a protein coding sequence between the N gene and the G gene. A non-coding sequence may or may not be present between the N gene and the G gene.

As used herein, a sequence that is positioned “immediately 5′” of another sequence means that the sequence is positioned 5′ to (i.e. upstream of) the other sequence without a protein coding sequence in between the two sequences. A non-coding sequence can be between the two sequences. For example, if an N gene is “immediately 5′” to a G gene, the N gene is positioned 5′ to the G gene, without a protein coding sequence between the N gene and the G gene. A non-coding sequence may or may not be present between the N gene and the G gene.

The term “isolated” as used herein with respect to nucleic acids, such as DNA or RNA, refers to molecules separated from other DNAs or RNAs, respectively that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments, which are not naturally occurring as fragments and would not be found in the natural state. The term “isolated” is also used herein to refer to polypeptides, which are isolated from other cellular proteins and is meant to encompass both purified and recombinant polypeptides. An “isolated cell” or “isolated population of cells” is a cell or population of cells that is not present in its natural environment.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

A “mutation” as used therein is a change in a DNA sequence resulting in an alteration from its natural state. The mutation can comprise a deletion and/or insertion and/or duplication and/or substitution of at least one deoxyribonucleic acid base such as a purine (adenine and/or thymine) and/or a pyrimidine (guanine and/or cytosine). Mutations may or may not produce discernible changes in the observable characteristics (phenotype) of an organism.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids. As used herein, nucleic acids include but are not limited to, all nucleic acid sequences which are obtained by any means available in the art, including, without limitation, recombinant means, i.e., the cloning of nucleic acid sequences from a recombinant library or a viral genome, using ordinary cloning technology and PCR™, and the like, and by synthetic means.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. There are numerous expression control sequences, including promoters which are native, constitutive, inducible and/or tissue-specific, are known in the art that may be used in the compositions of the invention. “Operably linked” should be construed to include RNA expression and control sequences in addition to DNA expression and control sequences.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence, which is required for expression of a gene product operably linked to the promoter/regulatory sequence. In some instances, this sequence may be the core promoter sequence and in other instances, this sequence may also include an enhancer sequence and other regulatory elements, which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

As used herein, the term “pharmaceutical composition” refers to a mixture of at least one compound useful within the invention with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the term “effective amount” or “therapeutically effective amount” means the amount of the virus like particle generated from vector of the invention which is required to prevent the particular disease condition, or which reduces the severity of and/or ameliorates the disease condition or at least one symptom thereof or condition associated therewith.

A “subject” or “patient,” as used therein, may be a human or non-human mammal. Non-human mammals include, for example, livestock and pets, such as ovine, bovine, porcine, canine, feline and murine mammals. Preferably, the subject is human. In some embodiments, the subject is a domestic pet or livestock. In some embodiments, the subject is a cat. In some embodiments, the subject is a dog. In some other embodiments, the subject is a ferret.

“Titers” are numerical measures of the concentration of a virus or viral vector compared to a reference sample, where the concentration is determined either by the activity of the virus, or by measuring the number of viruses in a unit volume of buffer. The titer of viral stocks are determined, e.g., by measuring the infectivity of a solution or solutions (typically serial dilutions) of the viruses, e.g., on HeLa cells using the soft agar method (see, Graham & Van Der eb (1973) Virology 52:456-467) or by monitoring resistance conferred to cells, e.g., G418 resistance encoded by the virus or vector, or by quantitating the viruses by UV spectrophotometry (see, Chardonnet & Dales (1970) Virology 40:462-477).

“Vaccination” refers to the process of inoculating a subject with an antigen to elicit an immune response in the subject, that helps to prevent or treat the disease or disorder the antigen is connected with. The term “immunization” is used interchangeably herein with vaccination.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In the present disclosure, the term “vector” includes an autonomously replicating virus.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to compositions and methods for generating vaccines against a lyssavirus. In some embodiments, the lyssavirus is a rabies virus.

Described herein is a vaccine against a lyssavirus, which is made using a rabies-based vector having a nucleic acid comprising (a) a nucleotide sequence encoding a nucleoprotein (N) of a rabies virus or a portion thereof and (b) a nucleotide sequence encoding a glycoprotein (G) (e.g., a RABV glycoprotein, a MOKV glycoprotein, a chimeric MOKV/RABV glycoprotein (G)) or a portion thereof positioned immediately 3′ to the nucleotide sequence encoding the nucleoprotein (N).

The lyssavirus vaccine described herein has the following advantages:
The construct contains a nucleic acid sequence encoding a glycoprotein (G) (e.g., a RABV glycoprotein, a MOKV glycoprotein, a chimeric MOKV/RABV glycoprotein (G)) or a portion thereof positioned immediately 3′ to a nucleotide sequence encoding a nucleoprotein (N) of a rabies virus. This results in higher expression levels of a recombinant rabies virus comprising the nucleic acid sequence in a host cell.
The RABV glycoprotein, some embodiments of the chimeric MOKV/RABV glycoprotein (G), or portions thereof present in the construct contains the so-called 333 mutation in the RABV G protein (Arg to Glu), which significantly reduces neurotropism of RABV.
The vaccine is expected to be highly attenuated with increased immunogenicity in the immunized host.

### Constructs

In one aspect, the present disclosure includes a nucleic acid comprising (a) a nucleotide sequence encoding a nucleoprotein (N) of a rabies virus or a portion thereof and (b) a nucleotide sequence encoding a glycoprotein (G) or a portion thereof positioned immediately 3′ to the nucleotide sequence encoding the nucleoprotein (N). In some embodiments, the glycoprotein (G) is selected from a RABV glycoprotein, a MOKV glycoprotein, and a chimeric MOKV/RABV glycoprotein. In one aspect, a nucleic acid is provided, the nucleic acid comprising (a) a nucleotide sequence encoding a nucleoprotein (N) of a rabies virus or a portion thereof and (b) a nucleotide sequence encoding a glycoprotein (G) or a portion thereof positioned immediately 3′ to the nucleotide sequence encoding the nucleoprotein (N), wherein the glycoprotein (G) is selected from a RABV glycoprotein, a MOKV glycoprotein, and a chimeric MOKV/RABV glycoprotein.

In one embodiment, the nucleic acid sequence encoding a nucleoprotein (N) of a rabies virus encodes the full nucleoprotein (N) of the rabies virus.

In some embodiments, the nucleotide sequence encoding the RABV glycoprotein, or the chimeric MOKV/RABV glycoprotein (G)) comprises a mutation that results in insertion of glutamic acid in place of arginine at position 333 of the RABV glycoprotein. In some embodiments, the mutation at position 333 significantly reduces the neurotropism of RABV.

In some embodiments, the nucleotide sequence encoding the chimeric MOKV/RABV glycoprotein comprises a nucleotide sequence encoding at least a portion of a MOKV glycoprotein and a nucleotide sequence encoding at least a portion of a RABV glycoprotein. In some embodiments, the chimeric MOKV/RABV glycoprotein comprises at least a portion of a MOKV glycoprotein and at least a portion of a RABV glycoprotein, wherein the at least a portion of a MOKV glycoprotein and at least a portion of a RABV glycoprotein are fused to form a chimeric protein. In some embodiments, the nucleotide sequence encoding the chimeric MOKV/RABV glycoprotein comprises a nucleotide sequence encoding at least one domain (e.g., clip, core, or flap) of a RABV glycoprotein and at least one domain (e.g., clip, core, or flap) of a MOKV glycoprotein. In some embodiments, the at least one domain of a RABV glycoprotein is selected from a clip, core, flap, transmembrane, and intracellular domain. In some embodiments, the at least one domain of a MOKV glycoprotein is selected from a clip, core, flap, transmembrane, and intracellular domain. In some embodiments, the chimeric MOKV/RABV glycoprotein comprises one or more of a clip, core, and flap domain of a RABV glycoprotein and one or more of a clip, core, and flap domain of a MOKV glycoprotein.

In one embodiment, the chimeric MOKV/RABV glycoprotein or portion thereof comprises a clip domain. The clip domain can be a MOKV glycoprotein or RABV glycoprotein clip domain. In another embodiment, the chimeric MOKV/RABV glycoprotein or portion thereof comprises a core domain. The core domain can be a MOKV glycoprotein or RABV glycoprotein core domain. In yet another embodiment, the chimeric MOKV/RABV glycoprotein or portion thereof comprises a flap domain. The flap domain can be a MOKV glycoprotein or RABV glycoprotein flap domain. In one embodiment, the MOKV/RABV glycoprotein or portion thereof comprises a clip domain, a core domain, and a flap domain. In one embodiment, the MOKV/RABV glycoprotein or portion thereof comprises, from N terminus to C terminus, respectively: a clip domain, a core domain, and a flap domain.

In one embodiment, the chimeric MOKV/RABV glycoprotein or portion thereof comprises an intracellular domain and a transmembrane domain. The intracellular domain can be a MOKV glycoprotein or RABV glycoprotein intracellular domain. The transmembrane domain can be a MOKV glycoprotein or RABV glycoprotein transmembrane domain.

In one embodiment, the chimeric MOKV/RABV glycoprotein or portion thereof comprises a clip domain, a core domain, a flap domain, a transmembrane domain, and an intracellular domain. In one embodiment, the MOKV/RABV glycoprotein or portion thereof comprises, from N terminus to C terminus, respectively: a clip domain, a core domain, a flap domain, a transmembrane domain, and an intracellular domain. In one embodiment, the nucleotide sequence encoding the chimeric MOKV/RABV glycoprotein or a portion thereof comprises a nucleotide sequence encoding a RABV glycoprotein clip domain, a nucleotide sequence encoding a MOKV glycoprotein core domain, and a nucleotide sequence encoding a RABV glycoprotein flap domain. In some embodiments, the chimeric MOKV/RABV glycoprotein or a portion thereof comprises a RABV glycoprotein clip domain, a MOKV glycoprotein core domain, and a RABV glycoprotein flap domain. In some embodiments, the chimeric MOKV/RABV glycoprotein or a portion thereof comprises, from the N terminus to C terminus, respectively: a RABV glycoprotein clip domain, a MOKV glycoprotein core domain, and a RABV glycoprotein flap domain. In some embodiments, the nucleotide sequence encoding the RABV glycoprotein flap domain comprises a mutation that results in insertion of glutamic acid in place of arginine at position 333 of the RABV glycoprotein.

In some embodiments, the nucleic acid sequence encoding the chimeric MOKV/RABV glycoprotein or a portion thereof further comprises a transmembrane domain and a cytoplasmic domain. In some embodiments, the transmembrane domain is a MOKV glycoprotein or RABV glycoprotein transmembrane domain. In some embodiments, the intracellular domain is a MOKV glycoprotein or a RABV glycoprotein intracellular domain. In some embodiments, the chimeric MOKV/RABV glycoprotein or a portion thereof comprises a RABV glycoprotein clip domain, a MOKV glycoprotein core domain, a RABV glycoprotein flap domain, a glycoprotein transmembrane domain, and a RABV glycoprotein intracellular domain. In some embodiments, the chimeric MOKV/RABV glycoprotein or a portion thereof comprises, from the N terminus to C terminus, respectively: a RABV glycoprotein clip domain, a MOKV glycoprotein core domain, a RABV glycoprotein flap domain, a glycoprotein transmembrane domain, and a RABV glycoprotein intracellular domain.

In another embodiment, the nucleic acid sequence encoding the chimeric MOKV/RABV glycoprotein (G) or a portion thereof comprises a nucleotide sequence encoding a MOKV glycoprotein clip domain, a nucleotide sequence encoding a RABV glycoprotein core domain, and a nucleotide sequence encoding a MOKV glycoprotein flap domain. In some embodiments, the chimeric MOKV/RABV glycoprotein or a portion thereof comprises a MOKV glycoprotein clip domain, a RABV glycoprotein core domain, and a MOKV glycoprotein flap domain. In some embodiments, the chimeric MOKV/RABV glycoprotein or a portion thereof comprises, from the N terminus to C terminus, respectively: a MOKV glycoprotein clip domain, a RABV glycoprotein core domain, and a MOKV glycoprotein flap domain.

In some embodiments, the nucleic acid sequence encoding the MOKV/RABV glycoprotein or a portion thereof further comprises a transmembrane domain and a cytoplasmic domain. In some embodiments, the chimeric MOKV/RABV glycoprotein or a portion thereof comprises a MOKV glycoprotein clip domain, a RABV glycoprotein core domain, a MOKV glycoprotein flap domain, a glycoprotein transmembrane domain, and a RABV glycoprotein intracellular domain. In some embodiments, the chimeric MOKV/RABV glycoprotein or a portion thereof comprises, from the N terminus to C terminus, respectively: a MOKV glycoprotein clip domain, a RABV glycoprotein core domain, a MOKV glycoprotein flap domain, a glycoprotein transmembrane domain, and a RABV glycoprotein intracellular domain.

In some embodiments, the nucleic acid comprises a MOKV glycoprotein nucleotide sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 2. In some embodiments, the nucleic acid comprises SEQ ID NO: 2. SEQ ID NO: 2 is reproduced below:

ATGAATCTCCCGTGTTTGACTGTTATCTTGATATTGTTTA
CTAAATACTCACTGGGAGAATTCCCTTTGTATACTATACC
AGAGAAAATAGAGAAGTGGACTCCGATTGATATGATACAC
CTGTCTTGTCCGAATAATCTGCTCTCCCAAGAGGAGGGCT
GCAACGCCGAGACACCTTTCACGTACTTTGAGCTCAAATC
AGGCTATTTGGCGCATCAGAAGGTTCAGGGGTTTACCTGC
ACTGGAGTAGTGAATGAAGCGGAAACCTATACCAACTTTG
TAGGATATGTTACAACAACGTTTAAGCGGAAACATTTCAG
ACCCACTGTAGCAGCATGCAGGGACGCATATAATTGGAAG
GTGAGCGGTGACCCTCGGTATGAGGAAAGTCTTCACACCC
CCTACCCTGATAGTTCCTGGTTGCGGACCGTCACCACAAC
CAAAGAGAGTCTGCTGATAATAAGTCCGTCCATTGTCGAG
ATGGATATTTATGGCCGGACTCTTCACAGCCCGATGTTCC
CGTCAGGGACGTGTAGCAAGCTGTACCCGTCAGTACCATC
CTGCAAGACAAACCACGACTATACCCTTTGGCTCCCCGAA
GATCCCTCTCTCAGCCTCATATGTGACATCTTTACTTCCT
CCAACGGCCAAAAAGCAATGAACGGATCACGGATATGTGG
CTTCAAAGATGAACGGGGATTTTACAGAAGTTTGAAAGGG
GCGTGCAAACTGACTTTGTGTGGCAAACCCGGCATCCGCT
TGTTCGATGGGACGTGGGTCAGCTTCGCCAGGCCGGACGT
GCATGTGTGGTGCACCCCCAACCAGCTGGTCAATATACAC
AATGACCGCCTTGATGAGATAGAACACTTGATTGTAGAGG
ACATTATTAAGCGCAGGGAGGAGTGCCTTGATACGCTTGA
AACAATCCTGATGAGCCAGAGCATAAGTTTTAGGCGGCTC
TCCCATTTTAGGAAACTCGTACCGGGTTATGGTAAAGCTT
ACACCGTCCTGAACGGCAGTTTGATGGAGACCAACGTCTA
TTACAAGAGGGTAGATAAGTGGACCGATATTTTGCCGTCT
AAAGGTTGTCTCAAGGTTGGGCAACAATGCATGGATCCTG
TTAAAGGTGTGTTCTTTAACGGGATAATTAAGGGTCCCGA
CGGACAGATTCTTATTCCTGAAATGCAAAGTGAGCAACTT
AAACAGCACATGGATCTCTTGAAAGCCGCTGTTTTCCCAT
TGCGACACCCTCTGATTGACAGAGGCGCTGTCTTTAAGAA
GGATGGTGACGCGGACGATTTCGTTGACCTCCATATGCCA
GACGTACACAAAAGTGTTAGTGATGTTGATCTCGGTTTGC
CTCATTGGGGTCTTTGGCTGATGATAGGTGCCGCTGTCGT
TGCATTTATGGTACTCATCTGTCTTCTCAGAGTTTGTTGC
AAGCGGGTGGGCCGCAGGCGGAGCCCCCACGCAACTCAAG
AAATACCACTGAGCGTTTCCAGTGCCCCGGTACCACGAGC
AAAAGTTGTCTCTAGCTGGGAGTCATACAAAGGGCTCCCC
GGTACTTGA

In one embodiment, the MOKV glycoprotein nucleotide sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 2 encodes an amino acid sequence.

In some embodiments, the amino acid sequence encoded by the MOKV glycoprotein has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 3. SE ID NO: 3 is reproduced below:

MNLPCLTVILILFTKYSLGEFPLYTIPEKIEKWTPIDMIH
LSCPNNLLSQEEGCNAETPFTYFELKSGYLAHQKVQGFTC
TGVVNEAETYTNFVGYVTTTFKRKHFRPTVAACRDAYNWK
VSGDPRYEESLHTPYPDSSWLRTVTTTKESLLIISPSIVE
MDIYGRTLHSPMFPSGTCSKLYPSVPSCKTNHDYTLWLPE
DPSLSLICDIFTSSNGQKAMNGSRICGFKDERGFYRSLKG
ACKLTLCGKPGIRLFDGTWVSFARPDVHVWCTPNQLVNIH
NDRLDEIEHLIVEDIIKRREECLDTLETILMSQSISFRRL
SHFRKLVPGYGKAYTVLNGSLMETNVYYKRVDKWTDILPS
KGCLKVGQQCMDPVKGVFFNGIIKGPDGQILIPEMQSEQL
KQHMDLLKAAVFPLRHPLIDRGAVFKKDGDADDFVDLHMP
DVHKSVSDVDLGLPHWGLWLMIGAAVVAFMVLICLLRVCC
KRVGRRRSPHATQEIPLSVSSAPVPRAKVVSSWESYKGLP
GT

In another embodiment, the nucleic acid comprises a chimeric MOKV/RABV glycoprotein nucleotide sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 4. In some embodiments, the nucleic acid comprises SEQ ID NO: 4. SEQ ID NO: 4 is reproduced below:

ATGGTCCCTCAGGCTCTGCTGTTCGTCCCACTGCTGGTCT
TCCCTCTGTGCTTTGGCAAGTTCCCTATCTACACTATTCC
CGACAAGCTGGGCCCCTGGTCTCCTATCGATATTCACCAT
CTGAGTTGCCCTAACAATCTGGTGGTCGAGGAGGAGGGCT
GCAACGCCGAGACACCTTTCACGTACTTTGAGCTCAAATC
AGGCTATTTGGCGCATCAGAAGGTTCAGGGGTTTACCTGC
ACTGGAGTAGTGAATGAAGCGGAAACCTATACCAACTTTG
TAGGATATGTTACAACAACGTTTAAGCGGAAACATTTCAG
ACCCACTGTAGCAGCATGCAGGGACGCATATAATTGGAAG
GTGAGCGGTGACCCTCGGTATGAGGAAAGTCTTCACACCC
CCTACCCTGATAGTTCCTGGTTGCGGACCGTCACCACAAC
CAAAGAGAGTCTGCTGATAATAAGTCCGTCCATTGTCGAG
ATGGATATTTATGGCCGGACTCTTCACAGCCCGATGTTCC
CGTCAGGGACGTGTAGCAAGCTGTACCCGTCAGTACCATC
CTGCAAGACAAACCACGACTATACCCTTTGGCTCCCCGAA
GATCCCTCTCTCAGCCTCATATGTGACATCTTTACTTCCT
CCAACGGCCAAAAAGCAATGAACGGATCACGGATATGTGG
CTTCAAAGATGAACGGGGATTTTACAGAAGTTTGAAAGGG
GCGTGCAAACTGACTTTGTGTGGCAAACCCGGCATCCGCT
TGTTCGATGGGACGTGGGTCAGCTTCGCCAGGCCGGACGT
GCATGTGTGGTGCACCCCCAACCAGCTGGTCAATCTGCAC
GACTTCAGGAGCGACGAGATCGAACATCTGGTGGTCGAGG
AACTGGTGCGAAAAAGGGAGGAATGTCTGGATGCCCTGGA
GTCCATCATGACTACCAAGAGCGTGAGCTTCAGGAGGCTG
TCTCACCTGCGAAAGCTGGTGCCCGGCTTCGGCAAAGCCT
ACACCATCTTTAACAAGACACTGATGGAAGCAGACGCCCA
TTATAAATCAGTGGAGACCTGGAATGAAATTCTGCCAAGC
AAGGGCTGCCTGCGGGTGGGCGGACGCTGTCACCCACATG
TGAACGGCGTCTTCTTTAATGGAATCATTCTGGGGCCCGA
CGGCAACGTGCTGATCCCTGAGATGCAGTCTAGTCTGCTG
CAGCAGCACATGGAGCTGCTGGAATCAAGCGTGATTCCTC
TGGTCCATCCACTGGCAGATCCCTCCACAGTGTTCAAAGA
CGGAGATGAGGCCGAAGACTTTGTGGAAGTCCACCTGCCT
GATGTGCATAACCAGGTGTCTGGCGTCGACCTGGGACTGC
CAAATTGGGGCAAGTACGTGCTGCTGAGTGCTGGAGCACT
GACTGCCCTGATGCTGATCATTTTCCTGATGACCTGCTGT
CGGCGCGTGAACAGAAGTGAGCCCACTCAGCACAATCTGC
GAGGAACCGGGAGAGAAGTGTCAGTCACACCTCAGAGCGG
GAAAATCATTAGTAGTTGGGAATCACATAAAAGCGGGGGC
GAGACCAGGCTGTGA

In one embodiment, the chimeric MOKV/RABV glycoprotein nucleotide sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 4 encodes an amino acid sequence. In some embodiments, the amino acid sequence encoded by the chimeric MOKV/RABV glycoprotein has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 5. SEQ ID NO: 5 is reproduced below:

MVPQALLFVPLLVFPLCFGKFPIYTIPDKLGPWSPIDIHH
LSCPNNLVVEEEGCNAETPFTYFELKSGYLAHQKVQGFTC
TGVVNEAETYTNFVGYVTTTFKRKHFRPTVAACRDAYNWK
VSGDPRYEESLHTPYPDSSWLRTVTTTKESLLIISPSIVE
MDIYGRTLHSPMFPSGTCSKLYPSVPSCKTNHDYTLWLPE
DPSLSLICDIFTSSNGQKAMNGSRICGFKDERGFYRSLKG
ACKLTLCGKPGIRLFDGTWVSFARPDVHVWCTPNQLVNLH
DFRSDEIEHLVVEELVRKREECLDALESIMTTKSVSFRRL
SHLRKLVPGFGKAYTIFNKTLMEADAHYKSVETWNEILPS
KGCLRVGGRCHPHVNGVFFNGIILGPDGNVLIPEMQSSLL
QQHMELLESSVIPLVHPLADPSTVFKDGDEAEDFVEVHLP
DVHNQVSGVDLGLPNWGKYVLLSAGALTALMLIIFLMTCC
RRVNRSEPTQHNLRGTGREVSVTPQSGKIISSWESHKSGG
ETRL

In some embodiments, the nucleic acid encodes a recombinant virus comprising a sequence encoding at least a portion of the genome of the rabies virus, wherein the at least a portion of the genome of the rabies virus comprises (a) a nucleotide sequence encoding a nucleoprotein (N) of a rabies virus or a portion thereof and wherein the nucleic acid sequence further comprises (b) a nucleotide sequence encoding a glycoprotein (G) or a portion thereof positioned immediately 3′ to the sequence encoding the nucleoprotein (N). In some embodiments, the glycoprotein (G) is selected from a RABV glycoprotein, a MOKV glycoprotein, a chimeric MOKV/RABV glycoprotein (G). In some embodiments, the nucleic acid encoding the recombinant virus comprises at least a portion of the BNSP333 vector. In some embodiments, the at least a portion of the genome of the rabies virus comprises (a) a nucleotide sequence encoding a nucleoprotein (N) of a rabies virus or a portion thereof, (c) a nucleotide sequence encoding a rabies virus phosphoprotein (P) or a portion thereof, (d) a nucleotide sequence encoding a rabies virus protein (M) or a portion thereof, and (e) a nucleotide sequence encoding rabies virus protein (L) or a portion thereof. In some embodiments, the at least a portion of the genome of the rabies virus comprises (b) a nucleotide sequence encoding a RABV glycoprotein (G) or a portion thereof. In some embodiments, the at least a portion of the genome of the rabies virus comprises (b) a nucleotide sequence encoding a MOKV glycoprotein (G) or a portion thereof.

In some embodiments, the nucleotide sequence encoding a nucleoprotein (N) of a rabies virus or a portion thereof is located in the native location of the N gene in the rabies virus genome. In one embodiment, the nucleotide sequence encoding the glycoprotein (G) (e.g., the RABV glycoprotein, the MOKV glycoprotein, the chimeric MOKV/RABV glycoprotein) or a portion thereof is inserted between the N gene and the P gene. In one embodiment, the nucleotide sequence encoding the glycoprotein (G) (e.g., the RABV glycoprotein, the MOKV glycoprotein, the chimeric MOKV/RABV glycoprotein) or a portion thereof is not in the native location of the RABV G gene in the rabies virus genome. In some embodiments, the nucleotide sequence does not comprise a sequence encoding the RABV glycoprotein (G) or a portion thereof in the native location of the G gene in the rabies virus genome.

In some embodiments, the recombinant virus is a SADB-19 rabies virus strain. In yet another embodiment, the nucleic acid encoding the recombinant virus is codon optimized for expression in a host cell. In one embodiment, the host cell is a mammalian cell.

In one embodiment, the nucleic acid comprises a non-coding region between the nucleotide sequence encoding the rabies virus nucleoprotein (N) or portion thereof (“sequence (a)”) and the nucleotide sequence encoding the glycoprotein (G) (e.g., the RABV glycoprotein, the MOKV glycoprotein, or the chimeric MOKV/RABV glycoprotein (G)) or portion thereof (“sequence (b)”). In one embodiment, the non-coding region between sequence (a) and sequence (b) is between about 3 nucleotides and about 100 nucleotides. In one embodiment, the non-coding region between sequence (a) and sequence (b) is between about 3 nucleotides and about 90 nucleotides. In one embodiment, the non-coding region between sequence (a) and sequence (b) is between about 3 nucleotides and about 80 nucleotides. In one embodiment, the non-coding region between sequence (a) and sequence (b) is between about 3 nucleotides and about 70 nucleotides. In one embodiment, the non-coding region between sequence (a) and sequence (b) is between about 3 nucleotides and about 60 nucleotides. In one embodiment, the non-coding region between sequence (a) and sequence (b) is between about 3 nucleotides and about 50 nucleotides. In one embodiment, the non-coding region between sequence (a) and sequence (b) is between about 10 nucleotides and about 50 nucleotides. In one embodiment, the non-coding region between sequence (a) and sequence (b) is between about 20 nucleotides and about 50 nucleotides. In one embodiment, the non-coding region between sequence (a) and sequence (b) is between about 30 nucleotides and about 50 nucleotides. In one embodiment, the non-coding region between sequence (a) and sequence (b) is between about 35 nucleotides and about 45 nucleotides. In one embodiment, the non-coding region between sequence (a) and sequence (b) is about 39 nucleotides.

In some embodiments, the nucleic acid further comprises (c) a nucleotide sequence encoding a rabies virus phosphoprotein (P) or a portion thereof (“sequence (c)”) positioned immediately 3′ to nucleotide sequence (b). In one embodiment, the isolated nucleic acid comprises a non-coding region between sequence (b) and sequence (c). In one embodiment, the non-coding region between sequence (b) and sequence (c) is between about 3 nucleotides and about 100 nucleotides. In one embodiment, the non-coding region between sequence (b) and sequence (c) is between about 3 nucleotides and about 90 nucleotides. In one embodiment, the non-coding region between sequence (b) and sequence (c) is between about 3 nucleotides and about 80 nucleotides. In one embodiment, the non-coding region between sequence (b) and sequence (c) is between about 3 nucleotides and about 70 nucleotides. In one embodiment, the non-coding region between sequence (b) and sequence (c) is between about 3 nucleotides and about 60 nucleotides. In one embodiment, the non-coding region between sequence (b) and sequence (c) is between about 10 nucleotides and about 60 nucleotides. In one embodiment, the non-coding region between sequence (b) and sequence (c) is between about 20 nucleotides and about 60 nucleotides. In one embodiment, the non-coding region between sequence (b) and sequence (c) is between about 30 nucleotides and about 60 nucleotides. In one embodiment, the non-coding region between sequence (b) and sequence (c) is between about 40 nucleotides and about 60 nucleotides. In one embodiment, the non-coding region between sequence (b) and sequence (c) is between about 45 nucleotides and about 50 nucleotides. In one embodiment, the non-coding region between sequence (b) and sequence (c) is about 48 nucleotides.

In some embodiments, the nucleic acid further comprises (d) a nucleotide sequence encoding a rabies virus matrix protein (M) or portion thereof (“sequence (d)”) positioned immediately 3′ to nucleotide sequence (c), wherein the nucleotide sequence encoding protein (M) is positioned immediately 5′ to (e) a nucleotide sequence encoding rabies virus polymerase protein (L) or portion thereof (“sequence (e)”). In one embodiment, the isolated nucleic acid comprises a non-coding region between sequence (c) and sequence (d). In one embodiment, the non-coding region between sequence (c) and sequence (d) is between about 1 nucleotide and about 50 nucleotides. In one embodiment, the non-coding region between sequence (c) and sequence (d) is between about 1 nucleotide and about 45 nucleotides. In one embodiment, the non-coding region between sequence (c) and sequence (d) is between about 1 nucleotide and about 40 nucleotides. In one embodiment, the non-coding region between sequence (c) and sequence (d) is between about 1 nucleotide and about 35 nucleotides. In one embodiment, the non-coding region between sequence (c) and sequence (d) is between about 1 nucleotide and about 30 nucleotides. In one embodiment, the non-coding region between sequence (c) and sequence (d) is between about 1 nucleotide and about 25 nucleotides. In one embodiment, the non-coding region between sequence (c) and sequence (d) is between about 1 nucleotide and about 20 nucleotides. In one embodiment, the non-coding region between sequence (c) and sequence (d) is between about 1 nucleotide and about 15 nucleotides. In one embodiment, the non-coding region between sequence (c) and sequence (d) is between about 5 nucleotides and about 15 nucleotides. In one embodiment, the non-coding region between sequence (c) and sequence (d) is between about 5 nucleotides and about 10 nucleotides. In one embodiment, the non-coding region between sequence (c) and sequence (d) is about 8 nucleotides.

In one embodiment, the nucleic acid comprises a non-coding region between sequence (d) and sequence (e). In one embodiment, the non-coding region between sequence (d) and sequence (e) is between about 50 nucleotides and about 700 nucleotides. In one embodiment, the non-coding region between sequence (d) and sequence (e) is between about 50 nucleotides and about 650 nucleotides. In one embodiment, the non-coding region between sequence (d) and sequence (e) is between about 50 nucleotides and about 600 nucleotides. In one embodiment, the non-coding region between sequence (d) and sequence (e) is between about 50 nucleotides and about 550 nucleotides. In one embodiment, the non-coding region between sequence (d) and sequence (e) is between about 50 nucleotides and about 500 nucleotides. In one embodiment, the non-coding region between sequence (d) and sequence (e) is between about 50 nucleotides and about 450 nucleotides. In one embodiment, the non-coding region between sequence (d) and sequence (e) is between about 50 nucleotides and about 400 nucleotides. In one embodiment, the non-coding region between sequence (d) and sequence (e) is between about 100 nucleotides and about 400 nucleotides. In one embodiment, the non-coding region between sequence (d) and sequence (e) is between about 150 nucleotides and about 400 nucleotides. In one embodiment, the non-coding region between sequence (d) and sequence (e) is between about 200 nucleotides and about 400 nucleotides. In one embodiment, the non-coding region between sequence (d) and sequence (e) is between about 250 nucleotides and about 400 nucleotides. In one embodiment, the non-coding region between sequence (d) and sequence (e) is between about 300 nucleotides and about 400 nucleotides. In one embodiment, the non-coding region between sequence (d) and sequence (e) is between about 350 nucleotides and about 400 nucleotides. In one embodiment, the non-coding region between sequence (d) and sequence (e) is between about 350 nucleotides and about 475 nucleotides. In one embodiment, the non-coding region between sequence (d) and sequence (e) is about 363 nucleotides.

In some embodiments, the nucleic acid comprises a nucleotide sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1. In some embodiments, the nucleic acid comprises SEQ ID NO: 1. SEQ ID NO: 1 is reproduced below:

ATGGATGCCGACAAGATTGTATTCAAAGTCAATAATCAGG
TGGTCTCTTTGAAGCCTGAGATTATCGTGGATCAATATGA
GTACAAGTACCCTGCCATCAAAGATTTGAAAAAGCCCTGT
ATAACCCTAGGAAAGGCTCCCGATTTAAATAAAGCATACA
AGTCAGTTTTGTCAGGCATGAGCGCCGCCAAACTTAATCC
TGACGATGTATGTTCCTATTTGGCAGCGGCAATGCAGTTT
TTTGAGGGGACATGTCCGGAAGACTGGACCAGCTATGGAA
TTGTGATTGCACGAAAAGGAGATAAGATCACCCCAGGTTC
TCTGGTGGAGATAAAACGTACTGATGTAGAAGGGAATTGG
GCTCTGACAGGAGGCATGGAACTGACAAGAGACCCCACTG
TCCCTGAGCATGCGTCCTTAGTCGGTCTTCTCTTGAGTCT
GTATAGGTTGAGCAAAATATCCGGGCAAAACACTGGTAAC
TATAAGACAAACATTGCAGACAGGATAGAGCAGATTTTTG
AGACAGCCCCTTTTGTTAAAATCGTGGAACACCATACTCT
AATGACAACTCACAAaATGTGTGCTAATTGGAGTACTATA
CCAAACTTCAGATTTT
TGGCCGGAACCTATGACATGTTTTTCTCCCGGATTGAGCA
TCTATATTCAGCAATCAGAGTGGGCACAGTTGTCACTGCT
TATGAAGACTGTTCAGGACTGGTATCATTTACTGGGTTCA
TAAAACAAATCAATCTCACCGCTAGAGAGGCAATACTATA
TTTCTTCCACAAGAACTTTGAGGAAGAGATAAGAAGAATG
TTTGAGCCAGGGCAGGAGACAGCTGTTCCTCACTCTTATT
TCATCCACTTCCGTTCACTAGGCTTGAGTGGGAAATCTCC
TTATTCATCAAATGCTGTTGGTCACGTGTTCAATCTCATT
CACTTTGTAGGATGCTATATGGGTCAAGTCAGATCCCTAA
ATGCAACGGTTATTGCTGCATGTGCTCCTCATGAAATGTC
TGTTCTAGGGGGCTATCTGGGAGAGGAATTCTTCGGGAAA
GGGACATTTGAAAGAAGATTCTTCAGAGATGAGAAAGAAC
TTCAAGAATACGAGGCGGCTGAACTGACAAAGACTGACGT
AGCACTGGCAGATGATGGAACTGTCAACTCTGACGACGAG
GACTACTTTTCAGGTGAAACCAGAAGTCCGGAGGCTGTTT
ATACTCGAATCATGATGAATGGAGGTCGACTAAAGAGATC
TCACATACGGAGATATGTCTCAGTCAGTTCCAATCATCAA
GCCCGTCCAAACTCATTCGCCGAGTTTCTAAACAAGACAT
ATTCGAGTGACTCATAACATGAAAAAAACTAACACCCCTC
CCGTACGGCCGCCACCATGGTCCCTCAGGCTCTGCTGTTC
GTCCCACTGCTGGTCTTCCCTCTGTGCTTTGGCAAGTTCC
CTATCTACACTATTCCCGACAAGCTGGGCCCCTGGTCTCC
TATCGATATTCACCATCTGAGTTGCCCTAACAATCTGGTG
GTCGAGGACGAAGGGTGTACCAACCTGAGCGGCTTCTCCT
ACATGGAGCTGAAAGTGGGATATATCCTGGCTATTAAGGT
CAACGGGTTCACATGCACTGGCGTGGTCACCGAGGCAGAA
ACCTACACAAATTTTGTGGGCTATGTCACCACAACTTTCA
AGAGGAAACACTTTAGACCAACACCCGACGCCTGTCGCGC
CGCTTACAACTGGAAGATGGCTGGCGATCCACGATATGAG
GAATCTCTGCACAATCCTTACCCAGACTATAGATGGCTGC
GGACTGTGAAGACCACAAAAGAGTCCCTGGTCATCATTTC
CCCTTCTGTCGCAGACCTGGATCCATACGATAGATCTCTG
CACAGTCGGGTGTTTCCCTCCGGAAAGTGCTCTGGGGTGG
CTGTCAGCTCCACTTACTGTAGCACCAACCATGATTATAC
AATCTGGATGCCAGAGAATCCCAGGCTGGGGATGAGCTGC
GACATTTTCACAAATTCCCGCGGCAAGCGAGCCTCAAAAG
GAAGCGAGACTTGTGGGTTTGTGGACGAAAGGGGACTGTA
TAAGAGCCTGAAAGGGGCTTGCAAGCTGAAACTGTGCGGC
GTGCTGGGACTGAGACTGATGGATGGCACCTGGGTCAGTA
TGCAGACATCAAACGAGACTAAGTGGTGCCCCCCTGACAA
ACTGGTGAATCTGCACGACTTCAGGAGCGACGAGATCGAA
CATCTGGTGGTCGAGGAACTGGTGCGAAAAAGGGAGGAAT
GTCTGGATGCCCTGGAGTCCATCATGACTACCAAGAGCGT
GAGCTTCAGGAGGCTGTCTCACCTGCGAAAGCTGGTGCCC
GGCTTCGGCAAAGCCTACACCATCTTTAACAAGACACTGA
TGGAAGCAGACGCCCATTATAAATCAGTGGAGACCTGGAA
TGAAATTCTGCCAAGCAAGGGCTGCCTGCGGGTGGGCGGA
CGCTGTCACCCACATGTGAACGGCGTCTTCTTTAATGGAA
TCATTCTGGGGCCCGACGGCAACGTGCTGATCCCTGAGAT
GCAGTCTAGTCTGCTGCAGCAGCACATGGAGCTGCTGGAA
TCAAGCGTGATTCCTCTGGTCCATCCACTGGCAGATCCCT
CCACAGTGTTCAAAGACGGAGATGAGGCCGAAGACTTTGT
GGAAGTCCACCTGCCTGATGTGCATAACCAGGTGTCTGGC
GTCGACCTGGGACTGCCAAATTGGGGCAAGTACGTGCTGC
TGAGTGCTGGAGCACTGACTGCCCTGATGCTGATCATTTT
CCTGATGACCTGCTGTCGGCGCGTGAACAGAAGTGAGCCC
ACTCAGCACAATCTGCGAGGAACCGGGAGAGAAGTGTCAG
TCACACCTCAGAGCGGGAAAATCATTAGTAGTTGGGAATC
ACATAAAAGCGGGGGCGAGACCAGGCTGTGAGCTAGCCAT
GAAAAAAACTAACACCCCTCCTTTCGAACCATCCCAAACA
TGAGCAAGATCTTTGTCAATCCTAGTGCTATTAGAGCCGG
TCTGGCCGATCTTGAGATGGCTGAAGAAACTGTTGATCTG
ATCAATAGAAATATCGAAGACAATCAGGCTCATCTCCAAG
GGGAACCCATAGAGGTGGACAATCTCCCTGAGGATATGGG
GCGACTTCACCTGGATGATGGAAAATCGCCCAACCATGGT
GAGATAGCCAAGGTGGGAGAAGGCAAGTATCGAGAGGACT
TTCAGATGGATGAAGGAGAGGATCCTAGCTTCCTGTTCCA
GTCATACCTGGAAAATGTTGGAGTCCAAATAGTCAGACAA
ATGAGGTCAGGAGAGAGATTTCTCAAGATATGGTCACAGA
CCGTAGAAGAGATTATATCCTATGTCGCGGTCAACTTTCC
CAACCCTCCAGGAAAGTCTTCAGAGGATAAATCAACCCAG
ACTACTGGCCGAGAGCTCAAGAAGGAGACAACACCCACTC
CTTCTCAGAGAGAAAGCCAATCATCGAAAGCCAGGATGGC
GGCTCAAATTGCTTCTGGCCCTCCAGCCCTTGAATGGTCG
GCTACCAATGAAGAGGATGATCTATCAGTGGAGGCTGAGA
TCGCTCACCAGATTGCAGAAAGTTTCTCCAAAAAATATAA
GTTTCCCTCTCGATCCTCAGGGATACTCTTGTATAATTTT
GAGCAATTGAAAATGAACCTTGATGATATAGTTAAAGAGG
CAAAAAATGTACCAGGTGTGACCCGTTTAGCCCATGACGG
GTCCAAACTCCCCCTAAGATGTGTACTGGGATGGGTCGCT
TTGGCCAACTCTAAGAAATTCCAGTTGTTAGTCGAATCCG
ACAAGCTGAGTAAAATCATGCAAGATGACTTGAATCGCTA
TACATCTTGCTAACCGAACCTCTCCCCTCAGTCCCTCTAG
ACAATAAAATCCGAGATGTCCCAAAGTCAACATGAAAAAA
ACAGGCAACACCACTGATAAAATGAACCTCCTACGTAAGA
TAGTGAAAAACCGCAGGGACGAGGACACTCAAAAATCCTC
TCCCGCGTCAGCCCCTCTGGATGACGATGACTTGTGGCTT
CCACCCCCTGAATACGTCCCGCTGAAAGAACTTACAGGCA
AGAAGAACATGAGGAACTTTTGTATCAACGGAAGGGTTAA
AGTGTGTAGCCCGAATGGTTACTCGTTCAGGATCCTGCGG
CACATTCTGAAATCATTCGACGAGATATATTCTGGGAATC
ATAGGATGATCGGGTTAGTCAAAGTGGTTATTGGACTGGC
TTTGTCAGGATCTCCAGTCCCTGAGGGCCTGAACTGGGTA
TACAAATTGAGGAGAACCTTTATCTTCCAGTGGGCTGATT
CCAGGGGCCCTCTTGAAGGGGAGGAGTTGGAATACTCTCA
GGAGATCACTTGGGATGATGATACTGAGTTCGTCGGATTG
CAAATAAGAGTGATTGCAAAACAGTGTCATATCCAGGGCA
GAGTCTGGTGTATCAACATGAACCCGAGAGCATGTCAACT
ATGGTCTGACATGTCTCTTCAGACACAAAGGTCCGAAGAG
GACAAAGATTCCTCTCTGCTTCTAGAATAATCAGATTATA
TCCCGCAAATTTATCACTTGTTTACCTCTGGAGGAGAGAA
CATATGGGCTCAACTCCAACCCTTGGGAGCAATATAACAA
AAAACATGTTATGGTGCCATTAAACCGCTGCATTTCATCA
AAGTCAAGTTGATTACCTTTACATTTTGATCCTCTTGGAT
GTGAAAAAAACTATTAACATCCCTCAAAAGACCCCGGTAA
CGTCCTTTCAACGATCCAAGTCCATGAAAAAAACTAACAC
CCCTCCCGTACCTAGCTTATAAAGTGCTGGGTCATCTAAG
CTTTTCAGTCGAGAAAAAAACATTAGATCAGAAGAACAAC
TGGCAACACTTCTCAACCTGAGACTTACTTCAAGATGCTC
GATCCTGGAGAGGTCTATGATGACCCTATTGACCCAATCG
AGTTAGAGGCTGAACCCAGAGGAACCCCCATTGTCCCCAA
CATCTTGAGGAACTCTGACTACAATCTCAACTCTCCTTTG
ATAGAAGATCCTGCTAGACTAATGTTAGAATGGTTAAAAA
CAGGGAATAGACCTTATCGGATGACTCTAACAGACAATTG
CTCCAGGTCTTTCAGAGTTTTGAAAGATTATTTCAAGAAG
GTAGATTTGGGTTCTCTCAAGGTGGGCGGAATGGCTGCAC
AGTCAATGATTTCTCTCTGGTTATATGGTGCCCACTCTGA
ATCCAACAGGAGCCGGAGATGTATAACAGACTTGGCCCAT
TTCTATTCCAAGTCGTCCCCCATAGAGAAGCTGTTGAATC
TCACGCTAGGAAATAGAGGGCTGAGAATCCCCCCAGAGGG
AGTGTTAAGTTGCCTTGAGAGGGTTGATTATGATAATGCA
TTTGGAAGGTATCTTGCCAACACGTATTCCTCTTACTTGT
TCTTCCATGTAATCACCTTATACATGAACGCCCTAGACTG
GGATGAAGAAAAGACCATCCTAGCATTATGGAAAGATTTA
ACCTCAGTGGACATCGGGAAGGACTTGGTAAAGTTCAAAG
ACCAAATATGGGGACTGCTGATCGTGACAAAGGACTTTGT
TTACTCCCAAAGTTCCAATTGTCTTTTTGACAGAAACTAC
ACACTTATGCTAAAAGATCTTTTCTTGTCTCGCTTCAACT
CCTTAATGGTCTTGCTCTCTCCCCCAGAGCCCCGATACTC
AGATGACTTGATATCTCAACTATGCCAGCTGTACATTGCT
GGGGATCAAGTCTTGTCTATGTGTGGAAACTCCGGCTATG
AAGTCATCAAAATATTGGAGCCATATGTCGTGAATAGTTT
AGTCCAGAGAGCAGAAAAGTTTAGGCCTCTCATTCATTCC
TTGGGAGACTTTCCTGTATTTATAAAAGACAAGGTAAGTC
AACTTGAAGAGACGTTCGGTCCCTGTGCAAGAAGGTTCTT
TAGGGCTCTGGATCAATTCGACAACATACATGACTTGGTT
TTTGTGTTTGGCTGTTACAGGCATTGGGGGCACCCATATA
TAGATTATCGAAAGGGTCTGTCAAAACTATATGATCAGGT
TCACCTTAAAAAAATGATAGATAAGTCCTACCAGGAGTGC
TTAGCAAGCGACCTAGCCAGGAGGATCCTTAGATGGGGTT
TTGATAAGTACTCCAAGTGGTATCTGGATTCAAGATTCCT
AGCCCGAGACCACCCCTTGACTCCTTATATCAAAACCCAA
ACATGGCCACCCAAACATATTGTAGACTTGGTGGGGGATA
CATGGCACAAGCTCCCGATCACGCAGATCTTTGAGATTCC
TGAATCAATGGATCCGTCAGAAATATTGGATGACAAATCA
CATTCTTTCACCAGAACGAGACTAGCTTCTTGGCTGTCAG
AAAACCGAGGGGGGCCTGTTCCTAGCGAAAAAGTTATTAT
CACGGCCCTGTCTAAGCCGCCTGTCAATCCCCGAGAGTTT
CTGAGGTCTATAGACCTCGGAGGATTGCCAGATGAAGACT
TGATAATTGGCCTCAAGCCAAAGGAACGGGAATTGAAGAT
TGAAGGTCGATTCTTTGCTCTAATGTCATGGAATCTAAGA
TTGTATTTTGTCATCACTGAAAAACTCTTGGCCAACTACA
TCTTGCCACTTTTTGACGCGCTGACTATGACAGACAACCT
GAACAAGGTGTTTAAAAAGCTGATCGACAGGGTCACCGGG
CAAGGGCTTTTGGACTATTCAAGGGTCACATATGCATTTC
ACCTGGACTATGAAAAGTGGAACAACCATCAAAGATTAGA
GTCAACAGAGGATGTATTTTCTGTCCTAGATCAAGTGTTT
GGATTGAAGAGAGTGTTTTCTAGAACACACGAGTTTTTTC
AAAAGGCCTGGATCTATTATTCAGACAGATCAGACCTCAT
CGGGTTACGGGAGGATCAAATATACTGCTTAGATGCGTCC
AACGGCCCAACCTGTTGGAATGGCCAGGATGGCGGGCTAG
AAGGCTTACGGCAGAAGGGCTGGAGTCTAGTCAGCTTATT
GATGATAGATAGAGAATCTCAAATCAGGAACACAAGAACC
AAAATACTAGCTCAAGGAGACAACCAGGTTTTATGTCCGA
CATACATGTTGTCGCCAGGGCTATCTCAAGAGGGGCTCCT
CTATGAATTGGAGAGAATATCAAGGAATGCACTTTCGATA
TACAGAGCCGTCGAGGAAGGGGCATCTAAGCTAGGGCTGA
TCATCAAGAAAGAAGAGACCATGTGTAGTTATGACTTCCT
CATCTATGGAAAAACCCCTTTGTTTAGAGGTAACATATTG
GTGCCTGAGTCCAAAAGATGGGCCAGAGTCTCTTGCGTCT
CTAATGACCAAATAGTCAACCTCGCCAATATAATGTCGAC
AGTGTCCACCAATGCGCTAACAGTGGCACAACACTCTCAA
TCTTTGATCAAACCGATGAGGGATTTTCTGCTCATGTCAG
TACAGGCAGTCTTTCACTACCTGCTATTTAGCCCAATCTT
AAAGGGAAGAGTTTACAAGATTCTGAGCGCTGAAGGGGAG
AGCTTTCTCCTAGCCATGTCAAGGATAATCTATCTAGATC
CTTCTTTGGGAGGGATATCTGGAATGTCCCTCGGAAGATT
CCATATACGACAGTTCTCAGACCCTGTCTCTGAAGGGTTA
TCCTTCTGGAGAGAGATCTGGTTAAGCTCCCAAGAGTCCT
GGATTCACGCGTTGTGTCAAGAGGCTGGAAACCCAGATCT
TGGAGAGAGAACACTCGAGAGCTTCACTCGCCTTCTAGAA
GATCCGACCACCTTAAATATCAGAGGAGGGGCCAGTCCTA
CCATTCTACTCAAGGATGCAATCAGAAAGGCTTTATATGA
CGAGGTGGACAAGGTGGAAAATTCAGAGTTTCGAGAGGCA
ATCCTGTTGTCCAAGACCCATAGAGATAATTTTATACTCT
TCTTAATATCTGTTGAGCCTCTGTTTCCTCGATTTCTCAG
TGAGCTATTCAGTTCGTCTTTTTTGGGAATCCCCGAGTCA
ATCATTGGATTGATACAAAACTCCCGAACGATAAGAAGGC
AGTTTAGAAAGAGTCTCTCAAAAACTTTAGAAGAATCCTT
CTACAACTCAGAGATCCACGGGATTAGTCGGATGACCCAG
ACACCTCAGAGGGTTGGGGGGGTGTGGCCTTGCTCTTCAG
AGAGGGCAGATCTACTTAGGGAGATCTCTTGGGGAAGAAA
AGTGGTAGGCACGACAGTTCCTCACCCTTCTGAGATGTTG
GGATTACTTCCCAAGTCCTCTATTTCTTGCACTTGTGGAG
CAACAGGAGGAGGCAATCCTAGAGTTTCTGTATCAGTACT
CCCGTCCTTTGATCAGTCATTTTTTTCACGAGGCCCCCTA
AAGGGATACTTGGGCTCGTCCACCTCTATGTCGACCCAGC
TATTCCATGCATGGGAAAAAGTCACTAATGTTCATGTGGT
GAAGAGAGCTCTATCGTTAAAAGAATCTATAAACTGGTTC
ATTACTAGAGATTCCAACTTGGCTCAAGCTCTAATTAGGA
ACATTATGTCTCTGACAGGCCCTGATTTCCCTCTAGAGGA
GGCCCCTGTCTTCAAAAGGACGGGGTCAGCCTTGCATAGG
TTCAAGTCTGCCAGATACAGCGAAGGAGGGTATTCTTCTG
TCTGCCCGAACCTCCTCTCTCATATTTCTGTTAGTACAGA
CACCATGTCTGATTTGACCCAAGACGGGAAGAACTACGAT
TTCATGTTCCAGCCATTGATGCTTTATGCACAGACATGGA
CATCAGAGCTGGTACAGAGAGACACAAGGCTAAGAGACTC
TACGTTTCATTGGCACCTCCGATGCAACAGGTGTGTGAGA
CCCATTGACGACGTGACCCTGGAGACCTCTCAGATCTTCG
AGTTTCCGGATGTGTCGAAAAGAATATCCAGAATGGTTTC
TGGGGCTGTGCCTCACTTCCAGAGGCTTCCCGATATCCGT
CTGAGACCAGGAGATTTTGAATCTCTAAGCGGTAGAGAAA
AGTCTCACCATATCGGATCAGCTCAGGGGCTCTTATACTC
AATCTTAGTGGCAATTCACGACTCAGGATACAATGATGGA
ACCATCTTCCCTGTCAACATATACGGCAAGGTTTCCCCTA
GAGACTATTTGAGAGGGCTCGCAAGGGGAGTATTGATAGG
ATCCTCGATTTGCTTCTTGACAAGAATGACAAATATCAAT
ATTAATAGACCTCTTGAATTGGTCTCAGGGGTAATCTCAT
ATATTCTCCTGAGGCTAGATAACCATCCCTCCTTGTACAT
AATGCTCAGAGAACCGTCTCTTAGAGGAGAGATATTTTCT
ATCCCTCAGAAAATCCCCGCCGCTTATCCAACCACTATGA
AAGAAGGCAACAGATCAATCTTGTGTTATCTCCAACATGT
GCTACGCTATGAGCGAGAGATAATCACGGCGTCTCCAGAG
AATGACTGGCTATGGATCTTTTCAGACTTTAGAAGTGCCA
AAATGACGTACCTATCCCTCATTACTTACCAGTCTCATCT
TCTACTCCAGAGGGTTGAGAGAAACCTATCTAAGAGTATG
AGAGATAACCTGCGACAATTGAGTTCTTTGATGAGGCAGG
TGCTGGGCGGGCACGGAGAAGATACCTTAGAGTCAGACGA
CAACATTCAACGACTGCTAAAAGACTCTTTACGAAGGACA
AGATGGGTGGATCAAGAGGTGCGCCATGCAGCTAGAACCA
TGACTGGAGATTACAGCCCCAACAAGAAGGTGTCCCGTAA
GGTAGGATGTTCAGAATGGGTCTGCTCTGCTCAACAGGTT
GCAGTCTCTACCTCAGCAAACCCGGCCCCTGTCTCGGAGC
TTGACATAAGGGCCCTCTCTAAGAGGTTCCAGAACCCTTT
GATCTCGGGCTTGAGAGTGGTTCAGTGGGCAACCGGTGCT
CATTATAAGCTTAAGCCTATTCTAGATGATCTCAATGTTT
TCCCATCTCTCTGCCTTGTAGTTGGGGACGGGTCAGGGGG
GATATCAAGGGCAGTCCTCAACATGTTTCCAGATGCCAAG
CTTGTGTTCAACAGTCTTTTAGAGGTGAATGACCTGATGG
CTTCCGGAACACATCCACTGCCTCCTTCAGCAATCATGAG
GGGAGGAAATGATATCGTCTCCAGAGTGATAGATCTTGAC
TCAATCTGGGAAAAACCGTCCGACTTGAGAAACTTGGCAA
CCTGGAAATACTTCCAGTCAGTCCAAAAGCAGGTCAACAT
GTCCTATGACCTCATTATTTGCGATGCAGAAGTTACTGAC
ATTGCATCTATCAACCGGATCACCCTGTTAATGTCCGATT
TTGCATTGTCTATAGATGGACCACTCTATTTGGTCTTCAA
AACTTATGGGACTATGCTAGTAAATCCAAACTACAAGGCT
ATTCAACACCTGTCAAGAGCGTTCCCCTCGGTCACAGGGT
TTATCACCCAAGTAACTTCGTCTTTTTCATCTGAGCTCTA
CCTCCGATTCTCCAAACGAGGGAAGTTTTTCAGAGATGCT
GAGTACTTGACCTCTTCCACCCTTCGAGAAATGAGCCTTG
TGTTATTCAATTGTAGCAGCCCCAAGAGTGAGATGCAGAG
AGCTCGTTCCTTGAACTATCAGGATCTTGTGAGAGGATTT
CCTGAAGAAATCATATCAAATCCTTACAATGAGATGATCA
TAACTCTGATTGACAGTGATGTAGAATCTTTTCTAGTCCA
CAAGATGGTTGATGATCTTGAGTTACAGAGGGGAACTCTG
TCTAAAGTGGCTATCATTATAGCCATCATGATAGTTTTCT
CCAACAGAGTCTTCAACGTTTCCAAACCCCTAACTGACCC
CTCGTTCTATCCACCGTCTGATCCCAAAATCCTGAGGCAC
TTCAACATATGTTGCAGTACTATGATGTATCTATCTACTG
CTTTAGGTGACGTCCCTAGCTTCGCAAGACTTCACGACCT
GTATAACAGACCTATAACTTATTACTTCAGAAAGCAAGTC
ATTCGAGGGAACGTTTATCTATCTTGGAGTTGGTCCAACG
ACACCTCAGTGTTCAAAAGGGTAGCCTGTAATTCTAGCCT
GAGTCTGTCATCTCACTGGATCAGGTTGATTTACAAGATA
GTGAAGACTACCAGACTCGTTGGCAGCATCAAGGATCTAT
CCAGAGAAGTGGAAAGACACCTTCATAGGTACAACAGGTG
GATCACCCTAGAGGATATCAGATCTAGATCATCCCTACTA
GACTACAGTTGCCTGTGA

In yet another aspect, the present disclosure relates to a recombinant virus encoded by any one of the nucleic acids described herein. In some embodiments, the recombinant virus is a recombinant rabies virus. In one embodiment, the nucleic acid comprises (a) a nucleotide sequence encoding a rabies virus nucleoprotein (N) or a portion thereof, and (b) a nucleotide sequence encoding a glycoprotein (G) (e.g., a RABV glycoprotein, a MOKV glycoprotein, a chimeric MOKV/RABV glycoprotein) or a portion thereof positioned immediately 3′ to the sequence encoding the nucleoprotein (N). In some embodiments, the nucleic acid comprises a nucleotide sequence encoding the full rabies virus nucleoprotein (N) and (b) a nucleotide sequence encoding the full RABV glycoprotein (G). In another embodiment, the nucleic acid comprises a nucleotide sequence encoding the full rabies virus nucleoprotein (N) and (b) a nucleotide sequence encoding the full MOKV glycoprotein (G). In yet another embodiment, the nucleic acid comprises a nucleotide sequence encoding the full rabies virus nucleoprotein (N) and (b) a nucleotide sequence encoding a chimeric MOKV/RABV glycoprotein (G). The chimeric MOKV/RABV glycoprotein (G) may be any MOKV/RABV glycoprotein described elsewhere herein. In some embodiments, the recombinant virus is encoded by a nucleic acid described herein.

In yet another aspect, the present disclosure relates to a vector comprising a nucleic acid comprising (a) a nucleotide sequence encoding a nucleoprotein (N) of a rabies virus or a portion thereof and (b) a nucleotide sequence encoding a RABV glycoprotein, a MOKV glycoprotein, a chimeric MOKV/RABV glycoprotein (G), or portion thereof positioned immediately 3′ to the nucleotide sequence encoding the nucleoprotein (N). In some embodiments, the vector comprises a nucleic acid described herein.

In one embodiment, vector comprises a nucleic acid having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1. In some embodiments, the vector comprises a nucleic acid comprising SEQ ID NO: 1.

In another embodiment, the vector comprising the nucleic acid has at least 85%, at least 90%, at least 91%, at least 92%, at least 93%, at least 94%, at least 95%, at least 96%, at least 97%, at least 98%, or at least 99% sequence identity to SEQ ID NO: 1.

### Pharmaceutical Compositions and Formulations

The vaccine of the invention may be formulated as a pharmaceutical composition. In some embodiments, the vaccine contains a live virus. In some embodiments, the vaccine contains deactivated viral particles. In some embodiments, the virus is a recombinant virus encoded by any one of the nucleic acid constructs as described herein.

Such a pharmaceutical composition may be in a form suitable for administration to a subject (i.e. mammal), or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In one embodiment, the pharmaceutical compositions useful for practicing the method of the invention may comprise an adjuvant. Non-limiting examples of suitable adjuvants are Freund's complete adjuvant, Freund's incomplete adjuvant, Quil A, Detox, ISCOMs, squalene, MPLA, and CpG or other activators of TLR or inflammasome. The pharmaceutical composition or vaccine composition can comprise any one or more of the adjuvants described herein.

Pharmaceutical compositions that are useful in the methods of the invention may be suitably developed for inhalation, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

The composition of the invention may comprise a preservative from about 0.005% to 2.0% by total weight of the composition. The preservative is used to prevent spoilage in the case of exposure to contaminants in the environment.

### Administration/Dosing

The regimen of administration may affect what constitutes an effective amount. For example, the nucleic acid of the invention may be administered to the subject (i.e. mammal) in a single dose, in several divided dosages, as well as staggered dosages may be administered daily or sequentially, or the dose may be continuously infused, or may be a bolus injection. Further, the dosages may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to treat the disease in the subject. An effective amount of the composition necessary to achieve the intended result will vary and will depend on factors such as the disease to be treated or prevented, the age, sex, weight, condition, general health and prior medical history of the subject being treated, and like factors well-known in the medical arts. In particular embodiments, it is especially advantageous to formulate the composition in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on the unique characteristics of the composition and the heterologous protein to be expressed, and the particular therapeutic effect to be achieved.

### Routes of Administration

One skilled in the art will recognize that although more than one route can be used for administration, a particular route can provide a more immediate and more effective reaction than another route. Routes of administration of any of the compositions of the invention include inhalation, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, electroporation and topical administration.

### Kits

In some embodiments a kit is provided for treating, preventing, or ameliorating a given disease, disorder or condition, or a symptom thereof, as described herein wherein the kit comprises: a) compositions as described herein; and optionally b) an additional agent or therapy as described herein. The kit can further include instructions or a label for using the kit to treat, prevent, or ameliorate the disease, disorder or condition. In yet other embodiments, the invention extends to kits assays for a given disease, disorder or condition, or a symptom thereof, as described herein. Such kits may, for example, contain the reagents from PCR or other nucleic acid hybridization technology (microarrays) or reagents for immunologically based detection techniques (e.g., ELISpot, ELISA).

### Virus Production

In yet another aspect, the present disclosure includes a method of increasing expression of a recombinant virus in a host cell. In one embodiment, the recombinant virus is a rabies virus. In some embodiments, the method comprises expressing in the host cell a nucleic acid sequence described herein. In some embodiments, the nucleic acid sequence has at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the nucleic acid sequence comprises SEQ ID NO: 1.

The recombinant virus can be produced in a host cell using methods known in the art, e.g., as described in Fisher et al., Cell Reports 32, 107920, Jul. 21, 2020. In some embodiments, the host cell is a mammalian cell. In one embodiment, the host cell is a human cell. In one embodiment, the host cell is a primate cell. In some embodiments, the host cell is a BSR cell (a derivative of baby hamster kidney cell line BHK-21). In some embodiments, the host cell is a VERO cell (African green monkey cell line). In another embodiment, the host cell is a human lung cell, e.g., human lung cell line BEAS-2b.

### Methods of Treatment

In one aspect, the present disclosure includes a method of generating an immune response against a lyssavirus in a subject in need thereof. In another aspect, the present disclosure includes a method of vaccinating a subject against a lyssavirus. In yet another aspect, the present disclosure includes a method of providing immunity against a lyssavirus in a subject. In yet another aspect, the present disclosure includes a method of treating and/or preventing a disease or disorder associated with a lyssavirus in a subject in need thereof. In yet another aspect, method of increasing immunogenicity against a lyssavirus in a subject in need thereof. In one embodiment, the lyssavirus is a rabies virus (RABV). In another embodiment, the lyssavirus is a Mokola virus (MOKV). In some embodiments, the method comprises administering to the subject an effective amount of a recombinant virus as described herein. In some embodiments, the recombinant virus is encoded by a nucleic acid described herein. In some embodiments, the method comprises administering to the subject an effective amount of a vaccine described herein. In some embodiments, the subject is a mammal. In some embodiments, the subject is a human.

Pharmaceutical compositions comprising the vaccine of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

The administration of the vaccine of the invention may be carried out in any convenient manner known to those of skill in the art. The vaccine of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally.

Subjects to which administration of the pharmaceutical compositions of the invention is contemplated include, but are not limited to, humans and other primates, mammals, and birds, including commercially relevant mammals and birds such as cattle, pigs, horses, sheep, chicken, ducks, cats, dogs, and ferrets.

In some embodiments, the subject is a domesticated animal. In some embodiments, the subject is a domestic pet. In some embodiments, the animal is a captive animal, e.g., an animal maintained in an exhibit or in a zoological park. In some embodiments, the animal is livestock. In some embodiments, the subject is a feline. In some other embodiments, the subject is a canine. In some embodiments, the subject is a cat.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in these experiments are now described.

### Materials and Methods

### Experimental Model and Subject Details

Swiss Webster mice (Charles River), age 6-10 weeks, were used in this study. All mice used were female except where noted. Mice used in this study were handled in adherence to the recommendations described in the Guide for the Care and Use of Laboratory Animals, and work was approved by the Institutional Animal Care and Use Committee (IACUC) of Thomas Jefferson University (TJU) under protocols 01886 and 01940. Mice were housed with up to five individuals per cage, under controlled conditions of humidity, temperature, and light (12 h light, 12 h dark cycles). Food and water were available ad libitum. Animal procedures were conducted under 3% isoflurane/O₂ gas anesthesia.

The following cell lines and their culture conditions were used in this work: mouse neuroblastoma (NA) cells were grown in RPMI (Corning) with 5% fetal bovine serum (FBS, Atlanta Biologicals) and 1× Penicillin/Streptomycin (Corning). The other cell lines were grown in DMEM (Corning) with 5% FBS and 1× Penicillin/Streptomycin: BSR cells (a derivative of the baby hamster kidney cell line BHK-21), the African green monkey cell line VERO, and the human lung cell line BEAS-2b. Cells were kept at 37° C. and 5% CO₂ during non-infectious growth and 34° C. with 5% CO₂ during infectious growth. Infected cell cultures were cultured in OptiPRO SFM (Life Technologies) unless otherwise noted.

### Method Details

### Structural Modeling and Chimeric Glycoprotein Design

To generate structural models of RABV and MOKV G, their amino acid sequences were threaded onto the pre-fusion structure of vesicular stomatitis virus (VSV) G. Three different modeling programs were used to increase the likelihood of pro-ducing an accurate model: I-TASSER, SWISS-MODEL (Waterhouse et al., 2018), and Phyre2. After analysis and delineation of the clip, core, and flap regions of the glycoproteins, the proposed chimeric G were also threaded onto VSV G to confirm the placement of the ectodomain regions.

cDNA Construction of Vaccine Vectors

To make the rRABV vector, a human codon-optimized RABV G (SAD B19 strain with R333E mutation, synthesized by Genscript USA) was inserted into the BNSPDG vector using the BsiWI and NheI restriction sites. Human codon optimization was selected in anticipation of downstream vaccine production in primate cells. To make the BNSP333-coMOKVG vector, MOKV G (MOKV.NIG68-RV4 strain, GenBank accession number HM623780, provided by Gene Tan) was inserted into the BNSP333 vector using the InFusion cloning kit (Clontech) and oligos CO-041 and CO-042. To make the rMOKV vector, the human codon-optimized MOKV G sequence was inserted into the BNSPDG vector using the NotI and NheI restriction sites, the NotI site having been cloned into the vector previously. To make the chimeric glycoproteins 1 and 2, fragments of codon optimized RABV G and MOKV G were first amplified by PCR using primers and cloned into a pCAGGS expression vector via InFusion cloning (Clontech). Three fragments were combined to make Chimeric G 1 (amplified using oligos CO-062 through CO-067) and four fragments were combined to make Chimeric G 2 (amplified using oligos CO-067 through CO-074). The chimeric Gs were then cloned into the BNSPDG vector using the NotI and NheI restriction sites to create rChimeral (later termed LyssaVax) and rChimera2. The correct sequences of all four plasmids were confirmed by Sanger sequencing.

### Recovery of Recombinant Vectors

Recombinant RABV were recovered as described previously. Briefly, X-tremeGENE 9 transfection reagent (Millipore Sigma) in Opti-MEM reduced serum medium (Life Technologies) was used to co-transfect the respective full-length viral cDNA clones along with the plasmids encoding RABV N, P, and L and the T7 RNA polymerase into BSR cells in T25 flasks. The supernatants of transfected cells were harvested after 7 days and the supernatants were analyzed for the presence of infectious virus by infecting fresh BSR cell cultures and immunostaining with FITC-conjugated anti-RABV N mAb (Fujirebio).

To confirm the glycoprotein sequence in the recovered viruses, the viruses were sequenced by the following method: BSR cells were infected at an MOI of 1 then incubated for 2 days. Media was removed and the PureLink RNA Mini Kit (Ambion) was used to lyse the cells and extract RNA. Using the SuperScript II Reverse Transcriptase (Invitrogen), sections of the viral genomes containing G were amplified out of the total RNA (primers RP951 and RP952). RT-PCR products were run on an 1% agarose gel and bands were excised and analyzed by Sanger sequencing using the same primers.

### Immunofluorescence

To analyze broadened reactivity of the chimeric glycoproteins, VERO cells grown on 15 mm coverslips (Fisherbrand) were transfected with pCAGGS vectors containing either RABV G, MOKV G, Chimeric G 1 or Chimeric G 2 using XtremeGene 9. Two days post-transfection, cells were fixed with 4% paraformaldehyde (PFA), blocked with PBS containing 5% FBS, and stained with either the human anti-RABV G mAb 4C12 conjugated to DyLight 488, mouse anti-MOKV G sera (from G. Tan), or mouse anti-RABV G sera (generated against BNSP333), each at 1:400 dilution and incubated for 2 h at RT. Coverslips were washed with PBS and samples stained with mouse sera were then stained with Cy3-conjugated goat anti-mouse IgG secondary at 1:200. After a 2 h incubation at RT, coverslips were washed and mounted onto glass slides with Vectashield Hard Set containing DAPI (Vector Laboratories). Images of slides were analyzed in ProgRes (Jenoptic) and Fiji software.

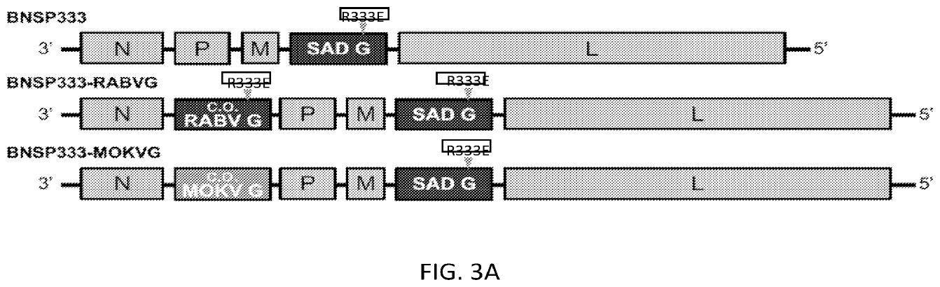

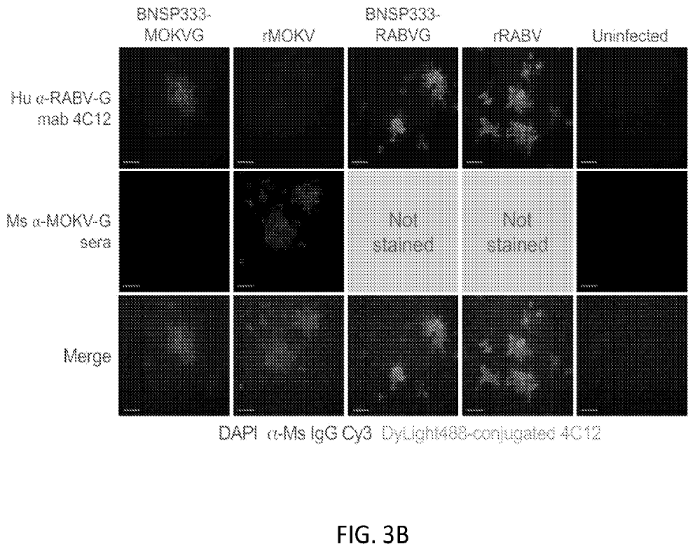

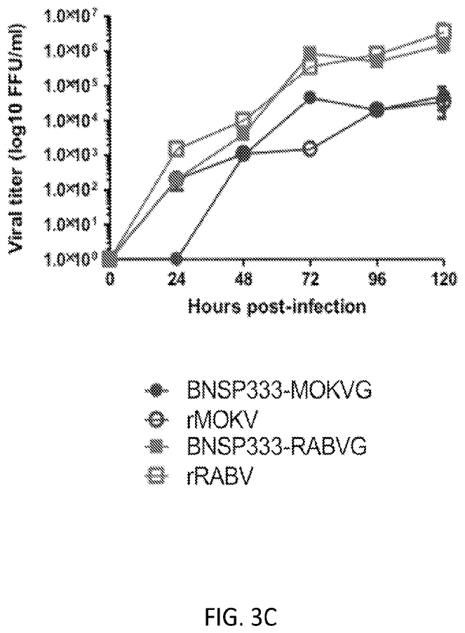

Immunofluorescence assays on infected cells were carried out in a similar manner, with the following difference: VERO cells were infected at MOI 0.01 with live virus (rRABV, rMOKV or LyssaVax in FIG. 2A; rRABV, rMOKV, BNSP333-MOKVG or BNSP333-RABVG in FIG. 3B) then fixed with 4% PFA 2 days post-transfection.

### Viral Growth Curve

Related to FIG. 3B. BSR cells were seeded in 6-well cell culture plates and incubated until 70% confluent. Cells were then infected at a MOI of 0.01 for 3 hours, washed 2× with PBS, and replenished with OptiPRO media (GIBCO). Samples of each well were collected every 24 h, stored at 4° C., then titered in triplicate.

### Purification and Inactivation of the Virus Particles

Large volumes of rRABV- and LyssaVax-containing supernatants were concentrated in a stirred 300 mL ultrafiltration cell (Millipore) and then purified over a 20% sucrose cushion in an SW32 Ti rotor (Beckman, Inc.) at 25,000 rpm for 1.5 h ay 4° C. rMOKV was purified similarly but without prior concentration in ultrafiltration cells. Virion pellets were resuspended in phosphate-buffered saline (PBS), and protein concentrations were determined using a bicinchoninic acid (BCA) assay kit (Pierce). The virus particles were inactivated with 50 mL per mg of particles of a 1:100 dilution of b-propiolactone (BPL) in cold water. The absence of infectivity was verified by inoculating BSR cells with 10 mg of BPL-inactivated viruses. After 4 days of incubation at 34° C., the cells were subcultured and 500 mL of supernatant was passaged on fresh BSR cells. Cultures were split 3 times, every 3 days. After the final growth period, cells were fixed and stained with a FITC-conjugated anti-RABV N mAb to confirm the absence of live virus.

### Protein Gel

To examine their purity, inactivated virus particles were diluted 1:1 in urea buffer (200 mM Tris-HCl [pH 6.8], 8 M urea, 5% sodium dodecyl sulfate (SDS), 0.1 mM ethylenediaminetetraacetic acid [pH 8], 0.03% bromophenol blue, and 0.5 M dithiothreitol) and denatured at 95° C. for 5 μm. Three micrograms of protein were resolved on a 10% SDS-polyacrylamide gel and stained with SYPRO Ruby Protein Gel Stain (Invitrogen) according to the manufacturer's specifications. The gel was then exposed under UV light for 430 ms.

### Pathogenicity Experiments

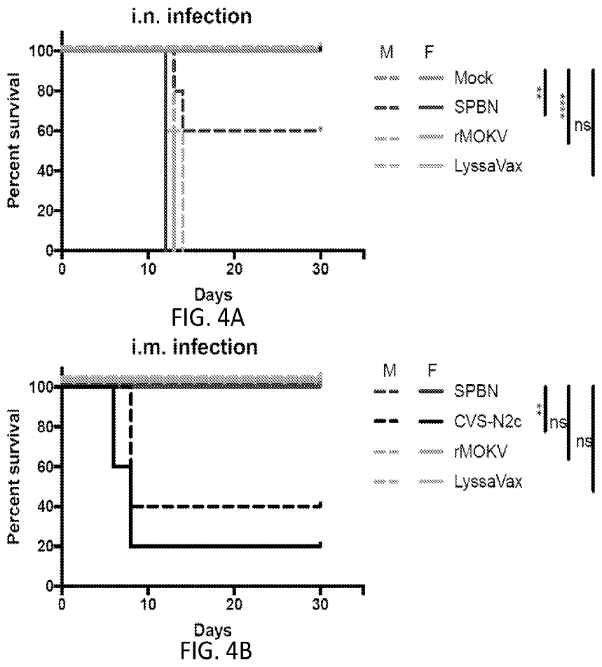

Related to FIGS. 4A and 4B. Four groups of Swiss Webster mice (Charles River, 5 male and 5 female per group, age 6 to 10 weeks) were intranasally (i.n.) infected with 10⁵ focus-forming units (FFU) of live virus diluted in 20 mL phosphate-buffered saline (PBS). The mice were weighed and monitored daily until day 21 post-infection and further monitored until day 30. Mice exhibiting signs of disease or that lost greater than 25% weight were euthanized. To assess a peripheral route of infection, 4 groups of Swiss Webster mice were intramuscularly (i.m.) infected with 10⁵ FFU of live virus diluted in 100 mL PBS, distributed equally to muscle of both hind limbs. The mice were weighed and monitored daily until day 21 post-infection and further monitored until day 28. Mice exhibiting signs of disease or that lost greater than 25% weight were euthanized. Survival was analyzed using the log-rank Mantel-Cox test in GraphPad Prism.

### Immunization and Challenge

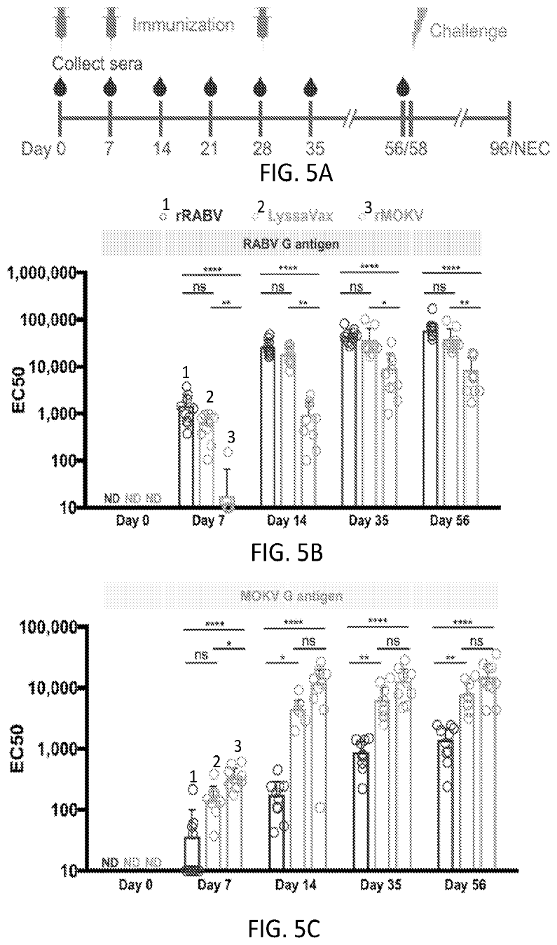

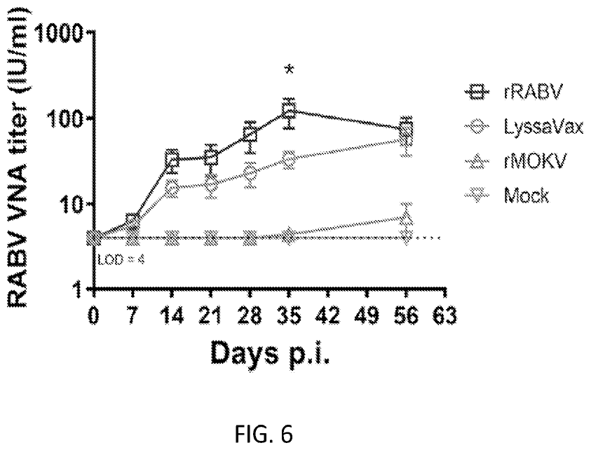

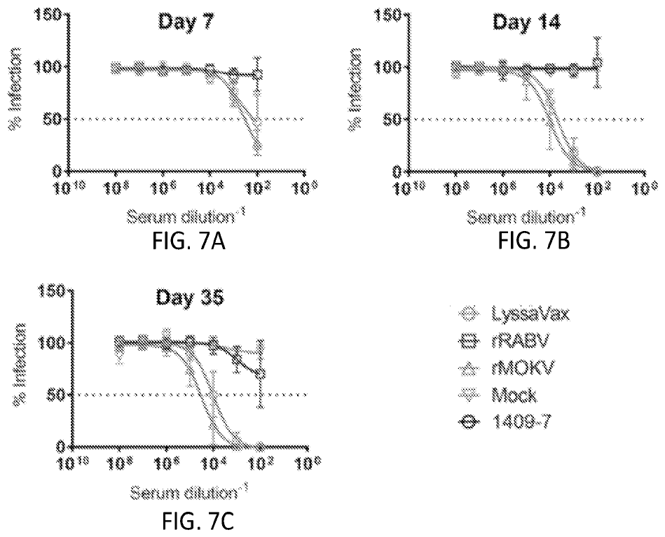

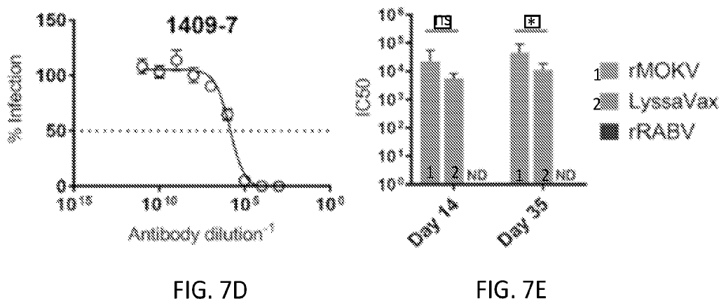

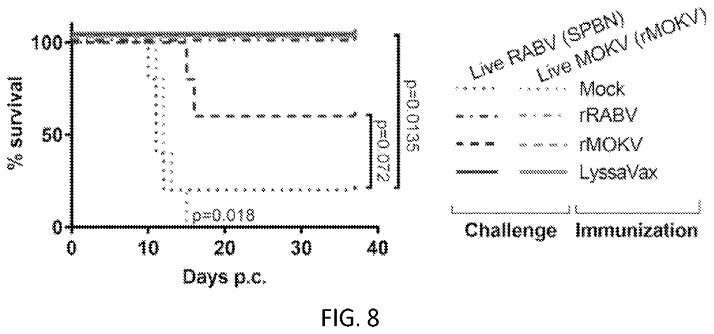

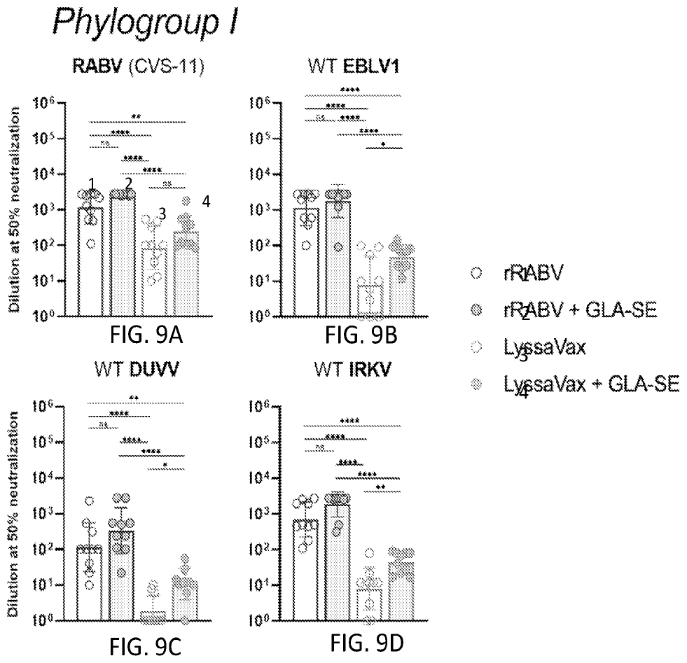

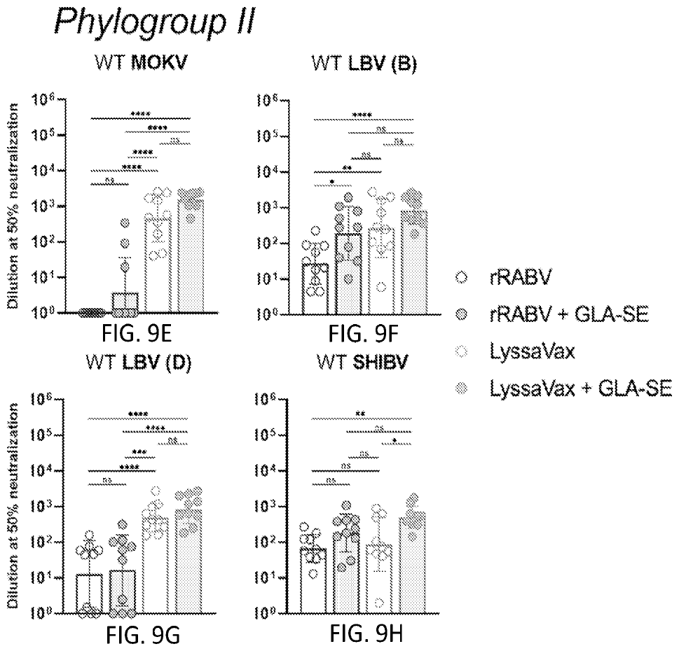

Swiss Webster mice (Charles River) were used in this study: groups of female mice, age 6 to 10 weeks, were immunized i.m. with 10 mg BPL-inactivated virus diluted in 100 mL phosphate-buffered saline (PBS) and distributed equally to muscle of both hind limbs. In groups which received glucopyranosyl lipid adjuvant-stable emulsion (GLA-SE, IDRI), 20 mL of 0.25 mg/ml adjuvant were included in the 100 mL total per mouse. Mice were immunized on days 0, 7, and 28. Blood was drawn (100 mL via the retro-orbital route) weekly and centrifuged at 10,000 rpm for 10 m for serum collection. Serum was analyzed from individual mice (unless noted). One set of mice (FIGS. 5A-5C; FIG. 6, FIGS. 7A-7E, and FIG. 8) was challenged on day 58 post-immunization (p.i.) with either SPNB or rMOKV. 10⁵ FFU of live virus were diluted in 20 mL PBS was administered i.n. The mice were weighed and monitored daily until day 21 post-infection and further monitored until day 37. Mice exhibiting signs of disease or that lost greater than 25% weight were euthanized. Survival was analyzed using the log-rank Mantel-Cox test in GraphPad Prism. The other set of mice (FIGS. 9A and 9B) was terminally bled via heart puncture on day 47 p.i. and euthanized.

### Production of Soluble RABV and MOKV G

To produce soluble RABV G, BEAS-2b cells were inoculated with VSVDG-GFP-RABVG at MOI 0.01. Three days post-infection, supernatant was collected, filtered through a 0.45 mm filter and concentrated by tangential flow filtration. Concentrated virus was purified over 20% sucrose cushion in a SW 32 Ti rotor (Beckman) at 25,000 rpm for 2 h. Pellets were resuspended in TEN buffer with 5% sucrose. To solubilize the glycoprotein, octyglucopyranoside (OGP, Fisher) was added to 2% final concentration and solution was incubated at room temperature with constant mixing for 30 μm. The suspension was spun at max speed in a benchtop centrifuge for 3 m to pellet debris. Pellets were treated with OGP in the same manner 2 more times. Pooled supernatants from all 3 extractions were spun in a SW 55 Ti rotor (Beckman) at 45,000 rpm for 1.5 h. Supernatant containing soluble G was analyzed for protein concentration by BCA (Pierce), and for purity by SDS-PAGE and western blot.

To produce soluble MOKV G, BEAS-2b cells were transfected with pCAGGS-coMOKVG using the XtremeGene 9 transfection re-agent (Millipore-Sigma). Two days post-transfection, cells were infected with MOKV G PTVS (described below), and 2 days post-infection, supernatant containing MOKVG pseudotyped VSV was collected. MOKV G was solubilized from the pseudotype virions in the same manner as RABV G.

### ELISA

To assess vaccine immunogenicity, mouse sera were analyzed by enzyme-linked immunosorbent assay (ELISA), probing for reactivity against either soluble RABV G or MOKV G (production described above). Mouse sera from days 0, 7, 14, 35, and 56 were analyzed individually in triplicate, except mock infected sera which was pooled. Immulon 96-well plates (Nunc) were coated with soluble G diluted in carbonate buffer (15 mM Na₂CO₃, 35 mM NaHCO₃[pH 9.5]). For RABV G, 50 ng in 100 mL buffer was used per well and for MOKV G, 25 ng in 50 mL per well. Plates were incubated overnight at 4° C. Plates were then washed 3 times with 300 mL per well of PBS containing 0.05% Tween 20 (PBST), then blocked with 5% milk in PBST (250 mL per well) for 2 h at RT, shaking. Plates were washed again, then coated with primary buffer (PBS with 0.5% bovine serum albumen), either 100 mL per well (RABV G plates) or 50 mL per well (MOKV G plates). Serum was diluted 3-fold down the plate in triplicate, starting at either 1:100 or 1:300, then plates were incubated overnight at 4° C. Plates were then washed 3 times and coated with PBST containing HRP-conjugated Goat anti-mouse IgG (H+L) at 1:10,000. After incubation for 2 h at RT, plates were washed and 200 mL of o-phenylenediamine dihydrochloride (OPD) substrate (Sigma) was added to each well. After a 15 m incubation, the reaction was stopped by the addition of 50 mL 3M H₂SO₄ per well. Optical density was determined at 490 nm (OD490). Individual mouse data were analyzed in GraphPad Prism using a sigmoidal dose-response fit (variable slope) to determine 50% effective concentration [EC₅₀]. Data from groups were also averaged (n=10) and plotted.

### RFFIT Neutralization Assays

Serum was first heat inactivated at 56° C. for 30 μm. Mouse sera from days 0, 7, 14, 21, 28, 35, 56 and at necropsy (surviving mice only) were analyzed individually in duplicate, except mock infected sera which was pooled. Rabies virus neutralizing activity was deter-mined using the rapid fluorescent focus inhibition test assay (RFFIT). Mouse neuroblastoma (NA) cells were seeded in 96-well plates 2 days prior to the assay (30,000 cells per well). Serum samples were 2-fold serially diluted in duplicate in 96-well plates, starting from at a dilution of 1:50 (unless otherwise noted) in 50 mL Opti-MEM (Life Technologies). The U.S. standard rabies immune globulin was used at 2 IU/ml. Working dilutions of RABV strain CVS-11 were prepared in Opti-MEM, and 5 mL the working dilution was added to each well containing diluted serum. Plates were incubated for 1 h at 34° C. Medium was removed from NA cells and diluted serum/virus mixtures were transferred to the cell plates. After 2 h incubation at 34° C., media was aspirated, replaced with fresh Opti-MEM, and plates were incubated for 22 h at 34° C. with 5% CO₂. Plates were then fixed with 80% acetone and stained with FITC-conjugated anti-RABV N antibody. 50% endpoint titers were calculated using the Reed-Muench method and converted to international units (IU) per milliliter by comparing to the standard.

### Generation of MOKV G PTVs

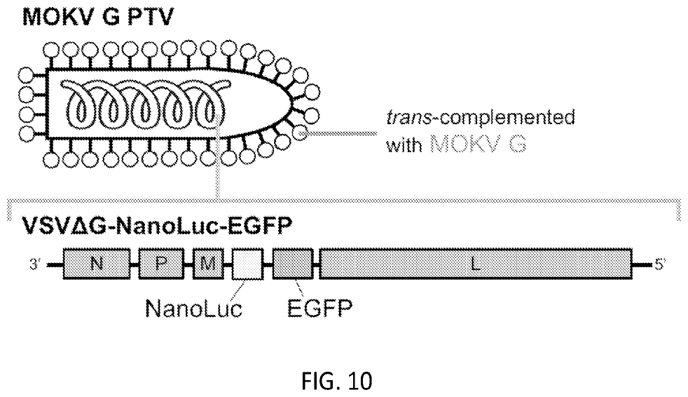

MOKV G pseudotype viruses (MOKV G PTVs) are single-round infectious particles comprised of MOKV Gs on the surface of the virion and a VSV genome lacking G and containing Nanoluciferase and EGFP (VSVDG-NanoLuc-EGFP) packaged within the virion (FIG. 10). To generate MOKV G PTVs, the human lung cell line BEAS-2B was first transfected with an expression vector containing human codon-optimized MOKV G (pCAGGS-coMOKVG) using X-tremeGENE 9 transfection reagent (Millipore Sigma). Two days post-transfection, cells were infected at an MOI of 1 with VSVDG-NanoLuc-EGFP pseudotyped with an irrelevant glycoprotein (Lassa fever virus glycoprotein complex) for initial infection. Two h post-infection, the inoculum was removed and cells were washed 3 times with PBS before media was replaced. Two days post-infection, cells were inspected for GFP expression under a fluorescent microscope and supernatant containing MOKV G PTVs was collected. MOKV G PTVs were passaged 3 times prior to use in the neutralization assay to ensure a pure population of virions.

### Pseudotype Virus Neutralization Assay

Serum was first heat inactivated at 56° C. for 30 μm. Individual mouse sera were analyzed in triplicate. Serum was diluted 10-fold start-ing at 1:100 dilution in Opti-MEM (Life Technologies) and 10⁴ MOKV G PTV particles were added to each dilution. The mix of sera/antibody plus virus was incubated for 1 h at 34° C. with 5% CO₂ and transferred to a previously seeded monolayer of VERO cells in a 96-well plate and further incubated for 2 h at 34° C. with 5% CO₂. Next, the virus/serum mix was replaced with DMEM. At 18-22 h post-infection, media was removed and cells were lysed with 40 mL 1× cell culture lysis buffer (Promega) and transferred to a white, opaque 96-well plate. The Nano-Glo Luciferase Assay System (Promega) was then used according to the manufacturer's recommendations. Relative luminescence units (RLU) were normalized to 100% infectivity signal as measured by no sera control (maximum signal) and signal from naive samples were background subtracted from experimental samples. Values that were above 100% infectivity were converted to 100%. Half maximal inhibition (IC₅₀) values were calculated by GraphPad Prism 7 using a nonlinear fit model (Log (inhibitor) versus normalized response variable slope). IC₅₀ data analyzed for statistical significance by the Mann-Whitney test.

### Microneutralization Assay

Sera from vaccinated mice were tested for VNAs against wild-type lyssaviruses using a microneutralization test. Briefly, serum was heat inactivated at 56° C. for 30 m, diluted 5-fold starting at 1:10 dilution in MEM supplemented with 10% FBS (CDC Division of Scientific Resources or Atlanta Biologics), and incubated at 37° C. for 90 m with 50 FFD50 of each of the following non-RABV lyssaviruses: RABV (CVS-11 strain), Irkut virus (IRKV), European bat lyssavirus 1 (EBLV1) Duvenhage virus (DUVV), Lagos bat virus (LBV, lineage B and lineage D), Shimoni bat virus (SHIBV), Mokola virus (MOKV). Individual mouse sera were analyzed in duplicate. Assays were performed either on 6 mm Teflon-coated slides or in 96-well cell culture plates. After incubation ˜50,000 cells/ml BSR (a clone of BHK-21) cells were added and mixtures were incubated at 37° C., 0.5% CO₂ for 20 to 44 h (depending on the virus used). Cells were then fixed with cold acetone and stained with FITC-conjugated anti-RABV N antibody (Fujirebio). Ten microscopic fields were observed for each dilution under fluorescent microscopy and 50% endpoint titers were calculated using the Reed-Muench method. Titers from pooled, naive (day 0) sera from each group were background subtracted from immune serum titers. Titers >1:10 were considered positive for VNAs. Microneutralization tests for LBV, MOKV, and SHIBV were performed under biosafety level 3 (BL3) conditions. The other tests were performed under BL2 conditions.

### Pseudotype Virus Neutralization Assay

Serum was first heat inactivated at 56° C. for 30 μm. Individual mouse sera were analyzed in triplicate. Serum was diluted 10-fold starting at 1:100 dilution in Opti-MEM (Life Technologies) and 10⁴ MOKV G PTV particles were added to each dilution. The mix of sera/antibody plus virus was incubated for 1 h at 34° C. with 5% CO₂ and transferred to a previously seeded monolayer of VERO cells in a 96-well plate and further incubated for 2 h at 34° C. with 5% CO₂. Next, the virus/serum mix was replaced with DMEM. At 18-22 h post-infection, media was removed and cells were lysed with 40 mL 1× cell culture lysis buffer (Promega) and transferred to a white, opaque 96-well plate. The Nano-Glo Luciferase Assay System (Promega) was then used according to the manufacturer's recommendations. Relative luminescence units (RLU) were normalized to 100% infectivity signal as measured by no sera control (maximum signal) and signal from naive samples were background subtracted from experimental samples. Values that were above 100% infectivity were converted to 100%. Half maximal inhibition (IC₅₀) values were calculated by GraphPad Prism 7 using a nonlinear fit model (Log (inhibitor) versus normalized response—variable slope). IC₅₀ data analyzed for statistical significance by the Mann-Whitney test.

### Quantification and Statistical Analysis

All statistical analysis was performed using GraphPad Prism software (version 8). ELISA EC₅₀ values were compared by Kruskal-Wallis tests and Dunn's multiple comparisons tests. RABV VNA titers were compared by two-way ANOVA and Tukey's multiple comparisons tests. MOKV pseudotype IC₅₀ values were compared using the Mann-Whitney test. Survival was analyzed using the log-rank Mantel-Cox test. Microneutralization data were analyzed using an ordinary one-way ANOVA test with Tukey's multiple comparisons. Where applicable, data was analyzed in a D'Agostino-Pearson omnibus normality test to check for normal distribution. Additional details of data processing are detailed in respective methods descriptions and additional details of statistical tests can be found in the figure legends.

The results of the experiments are now described.

### Results

Rabies is highly survivable with prompt administration of vaccines and antiserum. RABV vaccines are touted as one of the lowest cost but highest impact tradeoffs among vaccine-preventable infectious diseases (comparing procurement cost to Gavi, the Vaccine Alliance, and governments per death averted). Critically, disease from other lyssaviruses is not always prevented by RABV-based vaccines and biologics: protection against phylogroup II and III viruses is minimal, and lapses in coverage by post-exposure prophylaxis (PEP) have even been shown within phylogroup I, despite phylogenic proximity to RABV. Despite this, the available vaccines were developed solely against RABV. Investment in studying lyssaviruses and development of a pan-lyssavirus vaccine is currently lacking but would have a profound impact if or when a divergent lyssavirus emerges.

The fraction of disease burden shared by non-RABV lyssaviruses is unknown: the viral encephalitis and resulting symptoms from lyssaviruses are indistinguishable from RABV infections, and current diagnostic reagents based on the highly conserved nucleoprotein (N) cannot differentiate between lyssaviruses. Discriminatory diagnostics are rarely available for either human cases or surveillance in animal populations. Definitive evidence of a non-RABV lyssavirus infection can only be made in post-mortem analysis, and the methods (sequencing the viral genome or probing with species-specific antibodies) are not yet standardized. Seroprevalence studies in wildlife suggest lyssaviruses circulate in low but steady proportions compared with RABV. A small number of human deaths caused by six non-RABV lyssaviruses has been confirmed, but the actual number is likely higher.

Sporadic outbreaks of RABV and the consistent discovery of new lyssaviruses challenge the understanding of lyssavirus evolution and host switching. RABV likely originated as a bat-derived virus, then spread to terrestrial mammal reservoirs, notably dogs, numerous times. Canine RABV is now responsible for 95% of human RABV fatalities. Lyssaviruses circulate in bats with two notable exceptions where the reservoirs have not been identified: Ikoma virus (IKOV) and Mokola virus (MOKV). The possibility of further terrestrial adaptation and consequent increased risk to humans is of concern. MOKV, a divergent member of phylogroup II, was one of the first non-RABV lyssaviruses to be discovered, and lack of protection from RABV-based vaccines in animals has been well documented: for example, MOKV has been isolated from rabies-vaccinated domestic cats multiple times. Although rare cross-reactivity between RABV and MOKV has been observed, the current RABV vaccine is unlikely to provide protection against MOKV.

The unique attributes of rabies and its epidemiology call for economic factors to be considered during vaccine development. Despite its favorable cost-to-impact tradeoff, the current rabies vaccine is expensive, especially for rural populations in developing countries where transmission most often occurs. In addition, the current vaccine's long history of safety and reliability sets a high bar for new iterations. Therefore, in crafting a vaccine with broadened protection, the aim was to create a vaccine similar to current vaccines in composition and formulation: inactivated virions of a single strain. The glycoprotein (G) is the sole protein on the surface of the lyssavirus virions, and serum virus neutralizing antibodies (VNAs) against G are considered the primary correlate of protection against rabies disease. The disclosed vaccine therefore focuses on engineering the G.

Creating chimeric protein antigens is a well-established technique for modulating immune responses. The move toward “epitope-based” vaccines is an attractive approach in many efforts to make vaccines with increased safety, potency, and breadth. Previous attempts by other groups to create a chimeric G of RABV G and MOKV G, whether swapping antigenic sites or entire domains, were inconclusive or unsuccessful. Site switching is necessarily based on the known antigenic sites of RABV G. Five antigenic regions where VNAs bind were empirically mapped on RABV G, enabling deep understanding of neutralization mechanisms and the humoral response against RABV. However, detailed study of other lyssavirus Gs has not been carried out, so swapping these short regions may miss other important sites. This is evident from recent work in which antigenic sites were swapped between the Gs of RABV and Lagos bat virus (LBV): site II, considered a major site on RABV G, appears to also be immunodominant on LBV G, but the results of other sites are largely inconclusive. Swapping entire domains did not reliably produce infectious particles, likely because of the imperfect protein engineering caused by the lack of structural information at the time. Although transported to the cell surface and incorporated into budding virions, the chimeras failed to support viral replication, consistent with the lack of important structural determinants.

The vaccine described herein is based on a RABV vaccine strain and features a structurally designed chimeric lyssavirus glycoprotein containing domains from both RABV G and the highly divergent MOKV G. In mice, the inactivated vaccine elicits high titers of antibodies, which neutralize a panel of lyssaviruses, and protects against challenge with RABV and a recombinant MOKV.

### Example 1: Structure-Based Design of Chimeric Lyssavirus Glycoproteins

In designing a more broadly protective lyssavirus vaccine, MOKV G was initially inserted into a RABV vector already containing a native RABV G (BNSP333; FIG. 3A). This strategy has been successfully employed with various foreign viral Gs in the BNSP333 vector. However, the virus containing both Gs lost expression of MOKV G rapidly, as indicated by immuno-fluorescence (FIG. 3B). Furthermore, MOKV G alone or in addition to the native RABV G caused the vector to grow significantly slower (FIG. 3C). Therefore, a more technical strategy was pursued to create a single chimeric G, which would serve as the only glycoprotein supporting viral entry.

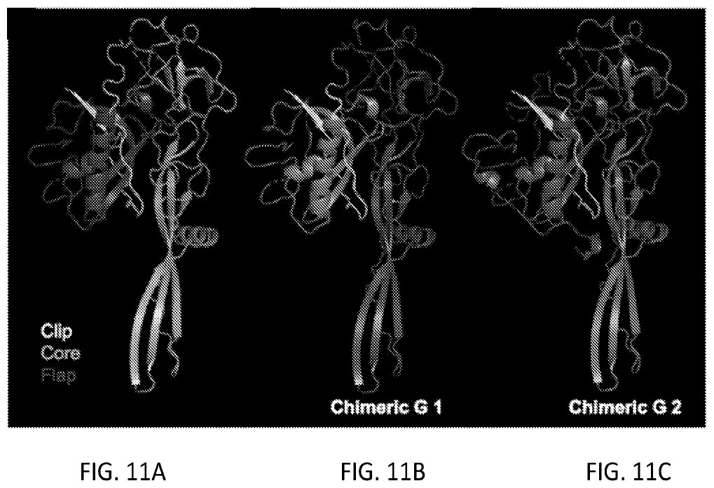

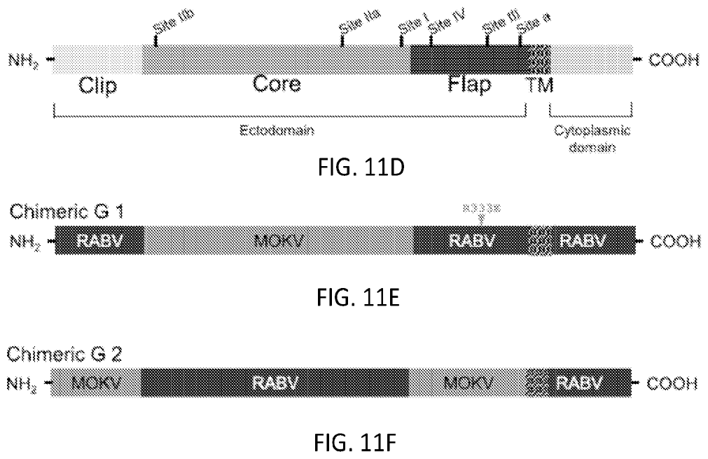

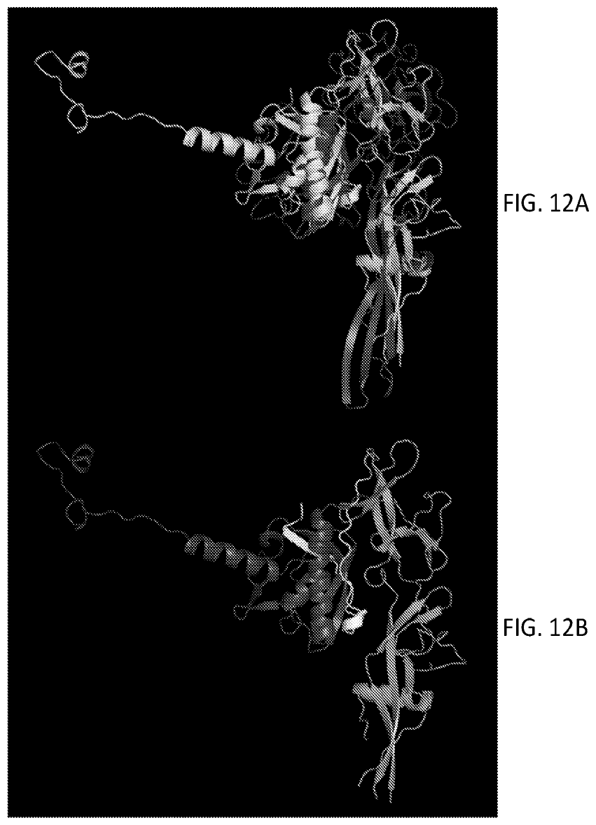

To design a chimeric G, structural models of lyssavirus Gs were created by threading their amino acid sequences onto the most closely related structure available at the time, that of the vesicular stomatitis virus (VSV) pre-fusion G. Three structural modeling programs: I-TASSER (Zhang, 2008), SWISS-MODEL, and Phyre2 (FIG. 11A) were used. Despite sharing only ˜18% sequence identity, VSV and RABV Gs appear to share conserved structural features, such as disulfide bonds, and VSV has previously been used to model RABV G. In all of the models, three subdomains were identified in the ectodomain: a “clip” that consists of a small hairpin-shaped region near the amino (N) terminus (yellow); a “core” that forms a large region containing a globular portion, beta sheets, and the putative fusion domain (orange); and a “flap,” the region near the transmembrane (TM) domain that associates closely with the clip and that contains the receptor binding domain (red) (FIGS. 11A and 11D). The structure of RABV G was recently solved at both low and high pH levels. Comparison between the high pH (pre-fusion) structure and the disclosed model shows similar positioning of the clip, core, and flap (FIGS. 12A and 12B), validating the use of structural modeling for designing chimeric proteins.

The clip, core, and flap subdomains formed the basis for building Chimeric G 1 and Chimeric G 2, which are comprised of alternating subdomains from RABV G and MOKV G (FIGS. 11B, 11C, 11E, and 11F). It was hypothesized that the design of a functional G protein requires the amino acid sequences of the clip and the flap to be derived from the same virus to reproduce optimal bonding interactions between these two moieties.

Other important features known about the well-studied RABV G were incorporated in the chimeric G designs. Extensive studies have mapped the majority of potently neutralizing mono-clonal antibodies (mAbs) to five antigenic sites on RABV G (FIG. 11D). The chimeric Gs share sites I and II from the same donor G, and sites III, IV, and minor site “a” from the other donor G, resulting in relatively balanced immunogenicity. The short, intracellular carboxy (C) terminal of RABV G interacts with the matrix protein (M) during viral budding and does not contribute to antigenicity, so it was maintained as RABV in both Chimeric G 1 and 2.

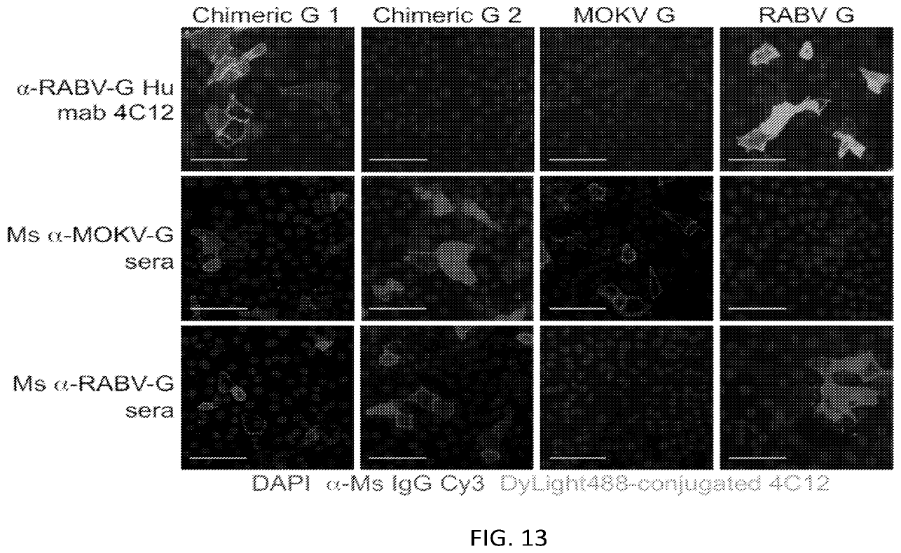

An immunofluorescence assay showed the chimeric Gs to successfully traffic to the cell surface and exhibit the anticipated antibody staining patterns (FIG. 13). Cells transiently expressing Chimeric G 1 or 2 were positively stained by polyclonal sera generated against RABV G or MOKV G, whereas cells expressing a wild-type (WT) G were stained only by their cognate sera. The human anti-RABV G mAb 4C12 binds in the flap region of RABV G and thus stains only Chimeric G 1.

### Example 2: Chimeric Glycoprotein is Functional and Supports Viral Replication

Four vaccines were then constructed in the BNSP RABV vector lacking its native G in the fourth position (BNSPDG; FIG. 2A). The G gene of interest was inserted into the second position: the vaccines rRABV and rMOKV contain the codon-optimized genes of RABV G or MOKV G, respectively, and the vaccines rChimeral and rChimera2 contain the respective chimeric Gs 1 and 2 (FIG. 2A). Placing of G in the second position of the genome instead of its native fourth position increases expression levels because of the transcription gradient exhibited by rhabdo-viruses. This increase in expression also contributes attenuation, which, despite proposed administration in an inactivated form, renders the vaccine safer to work with.

Multiple attempts to recover rChimera2 did not yield appreciable titers of infectious virus, suggesting that the chimeric G did not efficiently support viral spread. By contrast, rChimeral was successfully recovered, demonstrating the functionality of this G. An immunofluorescence assay confirmed that only cells infected with rChimeral, but not with either rRABV or rMOKV, exhibited dual staining with both anti-RABV G and anti-MOKV G reagents (FIG. 2B). Furthermore, the presence of foci indicates the virus's ability to spread from cell to cell mediated solely by the Chimeric G. Purified virions were analyzed by SDS-PAGE, which shows comparable incorporation of Chimeric G 1 into virions as compared with the control vaccines (FIG. 2C). rChimeral is henceforth referred to as LyssaVax.

### Example 3: LyssaVax is Apathogenic by Intramuscular and Intranasal Routes

Even though rabies vaccines are administered in an inactivated (killed) form to humans, safety is a necessary priority during production when the virus is live and concentrated. Therefore live LyssaVax was analyzed for any pathogenicity, comparing it with similar vectors containing the wild-type (WT) G protein from RABV or MOKV. LyssaVax was administered live by two inoculation routes, intranasal (i.n.) and intramuscular (i.m.), to assess potential pathogenicity in Swiss Webster mice (FIGS. 4A and 4B). Both male and female mice were used to ensure sex did not affect pathogenicity. LyssaVax was apathogenic both i.n., compared with the SPBN strain of RABV (FIG. 4A), and i.m., compared with the CVS-N2c strain (FIG. 4B).

### Example 4: LyssaVax Elicits High Titers of Antibodies Against Both RABV and MOKV

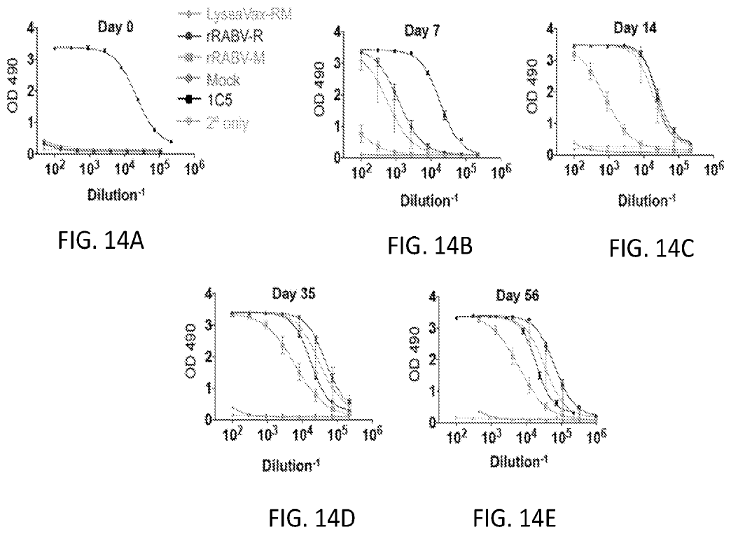

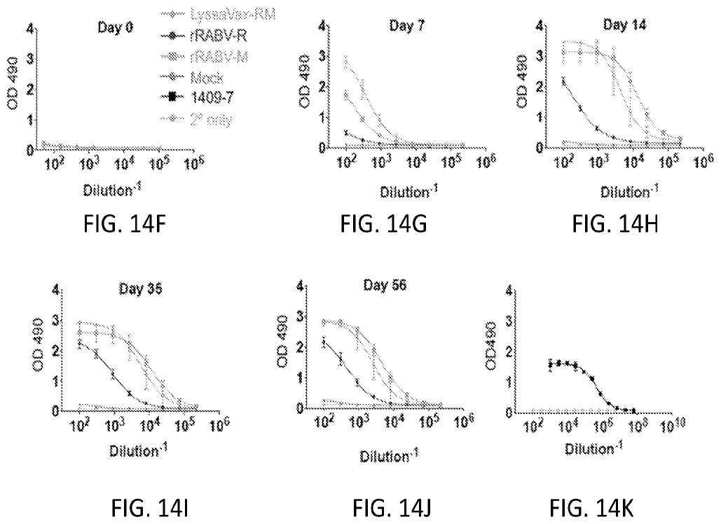

To assess immunogenicity, inactivated Lyssa-Vax was administered to groups of 10 Swiss Webster mice. Inactivated rRABV and rMOKV were administered individually as control vaccines. FIG. 5A displays the immunization and blood draw schedule (including challenge, discussed in the next section). Sera were analyzed by enzyme-linked immunosorbent assay (ELISA) against RABV G and MOKV G antigens. To avoid cross-reactivity with other RABV proteins, soluble Gs were produced, stripped, and purified from a recombinant VSV, which either expressed RABV G instead of VSV G or which lacked a G gene and was trans-complemented with MOKV G. mAbs against each protein were used to validate the antigen: the mouse anti-RABV G mAb 1C5 and the mouse mAb 1409-7, which cross-reacts with MOKV G (FIGS. 14A-14K).

Sera were tested to assess immunogenicity before immunization (day 0), 7 days following each immunization (days 7, 14, and 35) and just prior to challenge (day 56). Individual mouse half-maximal responses (EC₅₀s) are compared against RABV G (FIG. 3B) and MOKV G (FIG. 3C). Dilution curves of group averages are displayed in FIGS. 14A-14K. Sera from mice immunized with LyssaVax reacted strongly against both RABV and MOKV G antigens, nearly matching sera from cognate immunizations. EC₅₀s of RABV G-specific antibodies were not significantly different between LyssaVax and rRABV immune sera (FIG. 5B). EC₅₀s of MOKV G-specific antibodies from LyssaVax-immunized mice were significantly lower than rMOKV only at day 7 (p=0.0412; FIG. 5C). Sera from mock-immunized mice did not seroconvert to either antigen (FIGS. 14A-14K). Together, these data suggest that the chimeric G in LyssaVax is highly immunogenic and broadens the antigenicity of a single lyssavirus glycoprotein.

Interestingly, sera from rMOKV immune mice reacted to RABV G (dots/bars “3” in FIG. 5B) and sera from rRABV-immune mice reacted to MOKV G (dots/bars “1” in FIG. 5C), although both at significantly lower levels than LyssaVax sera. In both cases, titers increased over time (compared with mock-immunized sera), suggesting that these antibody responses were specific and being boosted by each subsequent immunization.

### Example 5: LyssaVax Elicits RABV Neutralizing Antibodies

Sera from mice immunized with LyssaVax neutralized RABV strain CVS-11 at comparable levels with rRABV control immune sera, as determined by the rapid fluorescent focus inhibition test (RFFIT) (FIG. 6; Table 1). When normalized to a rabies immunoglobulin standard, serum containing greater than 0.5 international units per milliliter (IU/mL) VNAs is considered adequate for protection. Neutralizing titers in all 10 LyssaVax-immunized mice reached >4 IU/mL by day 14 post-immunization (p.i.; after two vaccine inoculations on days 0 and 7). Titers continued to climb after the final immunization on day 28, peaking at an average of 57.2 IU/mL on day 56 p.i. Sera from mice immunized with the control vaccine, rRABV, yielded higher titers at each time point, but differed significantly (p=0.0342) only on day 35 p.i., when titers peaked, averaging 103.5 IU/mL. None of the mock-immunized groups exhibited virus neutralization capability, as determined by assaying pooled sera. These data demonstrate the potency of the chimeric G vaccine, because VNA titers are considered the most important correlate of protection.

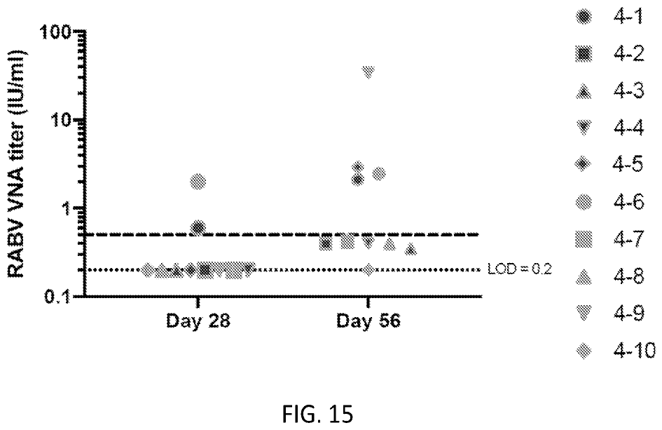

rMOKV immune sera did not neutralize CVS-11 above the 4 IU/mL level of detection initially used in the RFFIT, despite the high titers of cross-reactive antibodies observed in the RABV G ELISA. To address the possibility of VNA titers below 4 IU/mL, a follow-up assay was performed with larger dilutions of sera from days 28, 56, and 96 p.i., enabling a 0.2 IU/mL level of detection (FIG. 15). At day 28, only two mice exhibited titers above this new level of detection at 0.6 and 2 IU/mL, respectively. At day 56, 9/10 sera exhibited low levels of neutralization, with four mice achieving titers above the 0.5 IU/mL threshold for protection.

### Example 6: Antibodies Elicited by LyssaVax Neutralize MOKV G Pseudotypes

Unlike RABV, neither reference assays nor standards have been established for non-RABV lyssaviruses. Therefore, to assess the functionality of anti-MOKV G VNAs within the immunized mouse sera, sera was tested in a pseudotype neutralization assay. VSV lacking G and expressing NanoLuciferase and EGFP (VSVDG-NanoLuc-EGFP) was trans-complemented with MOKV G to create MOKV G pseudotype viruses (MOKV G PTVs; see FIG. 10 for schematic). Cells express NanoLuc and EGFP when infected with these single round infectious particles; thus, neutralization was measured as a reduction in luminescence. To account for background, luminescence was normalized to day 0 sera. Three time points were tested, each 1 week following a boost: days 7, 14, and 35 (FIGS. 7A-7E). Similar to the RFFIT, Lyssa-Vax-immune sera neutralized MOKV G PTVs at low concentrations, nearly as low as the control rMOKV. Of the two time points for which half-maximal inhibitory concentrations (IC₅₀) could be calculated, LyssaVax differed from rMOKV significantly only at day 35 (p=0.0133) (FIG. 7D). Sera from mice immunized with rRABV did not neutralize the MOKV G PTVs. Similar to the rMOKV sera in the RFFIT assay, high titers of cross-reactive sera seen in the ELISA did not correlate with functional VNA by day 35. Pooled sera from mock-immunized mice showed no neutralization.

TABLE 1
RABV neutralizing titers¹

| | Mouse | | | | | | | | | | |
|---|---|---|---|---|---|---|---|---|---|---|---|
| | ID | Day 0 | 7 | 14 | 21 | 28 | 35 | 56 | Challenge | 96 | |
| PBS | 1-1 | <0.2^{a} | No | <4^{b} | <4^{b} | <0.2^{a} | <4^{b} | <0.2^{a} | SPBN | Succumbed | |
| | 1-2 | | data | | | | | | rMOKV | Succumbed | |
| | 1-3 | | | | | | | | | Succumbed | |
| | 1-4 | | | | | | | | | 10.5 | |
| | 1-5 | | | | | | | | | Succumbed | |
| | 1-6 | | | | | | | | | Succumbed | |
| | 1-7 | | | | | | | | | Succumbed | |
| | 1-8 | | | | | | | | | Succumbed | |
| | 1-9 | | | | | | | | | Succumbed | |
| | 1-10 | | | | | | | | | Succumbed | |
| LyssaVax | 2-1 | <0.2^{a} | <4 | 13.8 | 9.9 | 26 | 19.1 | 33.8 | SPBN | 22.6 | 10.62 avg |
| | 2-2 | | <4 | 9.6 | 8.8 | 12.1 | 17.9 | 12.9 | | 10.3 | after SPBN |
| | 2-3 | | <4 | 5.2 | 6.1 | 11.1 | 9.6 | 6.1 | | 2 | challenge |
| | 2-4 | | <4 | 19.0 | 22.4 | 21.4 | 26.2 | 20.4 | | 7.8 | |
| | 2-5 | | <4 | 12.6 | 11.5 | 18.1 | 28.7 | 180 | | 10.4 | |
| | 2-6 | | <4 | 8.0 | 6.2 | 9.5 | 23.3 | 20.9 | rMOKV | 2.7 | 6.46 avg |
| | 2-7 | | <4 | 6.0 | 9.9 | 21.9 | 76.7 | 166.8 | | 20.9 | after rMOKV |
| | 2-8 | | <4 | 13.1 | 18 | 11.4 | 28.9 | 29.4 | | 2.7 | challenge |
| | 2-9 | | <4 | 29.5 | 16.2 | 11.4 | 27.5 | 20.2 | | 2.7 | |
| | 2-10 | | <4 | 37.7 | 60.4 | 86.2 | 76.2 | 81.3 | | 3.3 | |
| | Avg. | | | 15.45 | 16.94 | 22.91 | 33.41 | 57.18 | | 8.54 | |
| rRABV | 3-1 | <0.2^{a} | <4 | 24.5 | 17.7 | 25.6 | 34.9 | 9.9 | SPBN | 5.3 | 83.76 avg |
| | 3-2 | | <4 | 27.6 | 18.4 | 29 | 67 | 37.4 | | 137.8 | after SPBN |
| | 3-3 | | <4 | 12.0 | 6.2 | 9.1 | 28.4 | 34 | | 78.0 | challenge |
| | 3-4 | | <4 | 8.1 | 5.3 | 18.2 | 85.1 | 7.5 | | 130.1 | |
| | 3-5 | | <4 | 24.2 | 20.2 | 50.4 | 27.5 | 11.6 | | 67.6 | |
| | 3-6 | | 13.9 | 52.8 | 61 | 94.6 | 101.2 | 115.1 | rMOKV | 190.2 | 143 avg |
| | 3-7 | | <4 | 22.1 | 20.8 | 46.3 | 173.7 | 99.6 | | 196.4 | after rMOKV |
| | 3-8 | | <4 | 23.8 | 18.3 | 34 | 60 | 16.8 | | 36.7 | hallenge |
| | 3-9 | | 10.3 | 19.7 | 31 | 59.7 | 140 | 257.2 | | 138.0 | |
| | 3-10 | | 11.2 | 117.1 | 152.4 | 286.1 | 317.4 | 163.3 | | 153.8 | |
| | Avg. | | | 33.19 | 35.13 | 65.3 | 103.5 | 75.24 | | 113.39 | |
| rMOKV | 4-1 | <0.2^{a} | <4 | <4 | <4 | 0.6^{c} | <4 | 2.12^{c} | SPBN | 1.0^{c} | 2.03 avg |
| | 4-2 | | <4 | <4 | <4 | >0.2^{c} | <4 | 0.4^{c} | | Succumbed | after SPBN |
| | 4-3 | | <4 | <4 | <4 | >0.2^{c} | <4 | 0.35^{c} | | Succumbed | challenge |
| | 4-4 | | <4 | <4 | <4 | >0.2^{c} | <4 | 0.4^{c} | | 1.4^{c} | |
| | 4-5 | | <4 | <4 | <4 | >0.2^{c} | <4 | 2.95^{c} | | 3.7^{c} | |
| | 4-6 | | <4 | <4 | <4 | 2.0^{c} | <4 | 2.46^{c} | rMOKV | 1.4^{c} | 0.88 avg |
| | 4-7 | | <4 | <4 | <4 | >0.2^{c} | <4 | 0.4^{c} | | 0.7^{c} | after rMOKV |
| | 4-8 | | <4 | <4 | <4 | >0.2^{c} | <4 | 0.42^{c} | | 1.2^{c} | Challenge |
| | 4-9 | | <4 | <4 | <4 | >0.2^{c} | 8.2 | 33.96^{c} | | 0.9^{c} | |
| | 4-10 | | <4 | <4 | <4 | >0.2^{c} | No | >0.2^{c} | | <0.2^{c} | |
| | Avg. | | | | | | data | 4.37 | | 1.31 | |

¹Related to FIG. 6. RABV neutralizing titers in IU/ml as determined by RFFIT. The 4 immunogen groups are labeled in the first column: mock immunization with PBS, and immunization with rRABV, rMOKV, and LyssaVax. On day 58 after the start of immunizations, 5 mice from each group were challenged with either live SPBN or live rMOKV. Level of detection (LOD) ranged from 0.2 IU/ml to 4 IU/ml based on starting serum dilution (1:5 to 1:50, respectively). All samples tested in duplicate except individual serum samples tested 1:5, which were tested in singlet. All values listed in IU/ml.
^{a}Sera pooled, 1:5 starting dilution (LOD 0.2 IU/ml)
^{b}Sera pooled, 1.50 starting dilution (LOD 4 IU/ml)
^{c}Sera tested in singlet, 1:5 starting dilution (LOD 0.2 IU/ml)

### Example 7: LyssaVax Protects Against Lethal Challenge of Both RABV and rMOKV

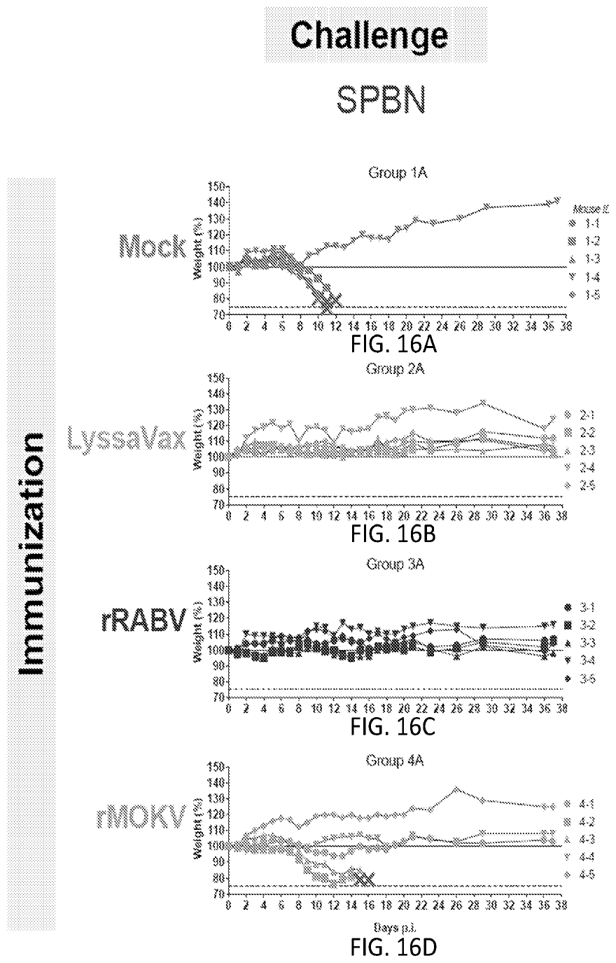

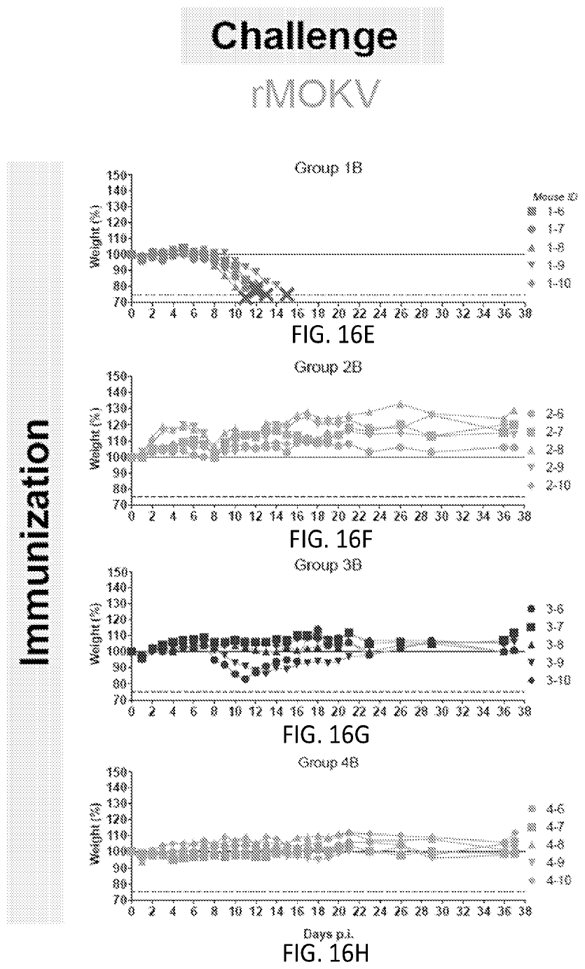

The vaccinated mice were challenged at 58 days p.i. (see schedule in FIG. 5A). The 10 mice per immunization group (LyssaVax, rRABV, rMOKV, and PBS mock) were split into two subgroups and challenged with 10⁵ focus-forming units (FFUs) of either live RABV (SPBN strain) or live rMOKV i.n. (FIG. 8 and FIGS. 16A-16H). Mock-immunized mice lost weight and were euthanized by day 12 post-challenge (p.c.) for SPBN and day 15 p.c. for rMOKV. One animal (mouse 1-4) challenged with SPBN survived and developed RABV neutralizing titers (Table 1). By contrast, all mice immunized with LyssaVax maintained weight and were protected against the live virus challenges. Mice immunized with the control vaccines were also protected against challenge with their cognate live virus: rRABV immune mice survived challenge by SPBN, and rMOKV immune mice survived live rMOKV challenge. Strikingly, some mice survived non-cognate challenge as well: three rMOKV immune mice survived SPBN challenge, and all five rRABV immune mice survived rMOKV challenge, although two mice (3-6 and 3-9) lost weight and recovered. Survival of these mice with low or negligible titers of cross-neutralizing antibodies may suggest alternate mechanisms of protection.

### Example 8: Antibodies Elicited by LyssaVax Neutralize Diverse WT Lyssaviruses

Because LyssaVax is composed of two component lyssavirus Gs, sera elicited by LyssaVax was tested to see if it cross-neutralized non-component viruses. The TLR-4 agonist glucopyranosyl lipid adjuvant-stable emulsion (GLA-SE) was also included as an adjuvant in some groups. GLA-SE has been shown to increase the magnitude and breadth of humoral immune responses and is currently in clinical trials. Four groups of mice were immunized with either rRABV or LyssaVax, with or without GLA-SE, following the same schedule in FIG. 5A. Sera from day 47 p.i. were tested in a micro-neutralization assay against a panel of WT lyssaviruses spanning two phylogroups: RABV, European bat lyssavirus 1 (EBLV1), Irkut virus (IRKV), and Duvenhage virus (DUVV) from phylogroup I (FIG. 9A); and MOKV, Shimoni bat virus (SHIBV), Lagos bat virus B (LBV-B), and LBV-D from phylogroup II (FIG. 9B).

Among phylogroup I viruses (FIG. 9A), all sera from the four groups neutralized RABV, as expected. Immune sera from rRABV with or without GLA-SE also cross-neutralized EBLV1, DUVV, and IRKV, consistent with cross-reactivity within phylogroups previously reported. Neutralizing titers from LyssaVax with or without GLA-SE were significantly lower than those of rRABV with or without adjuvant. However, including GLA-SE with LyssaVax raised the average neutralizing titer for all four viruses tested (significantly so for DUVV and IRKV), as compared with unadjuvanted LyssaVax. For DUVV, only 3/10 of the unadjuvanted LyssaVax-immune samples were neutralizing, whereas 9/10 of LyssaVax+GLA-SE samples neutralized.

Among phylogroup II viruses (FIG. 9B), sera from LyssaVax, both with and without adjuvant, neutralized WT viruses at significantly higher titers than sera from rRABV (with or without adjuvant). Strikingly, of the phylogroup II viruses, only WT MOKV was not neutralized by rRABV-immune sera, and only three samples exhibited neutralizing titers above baseline in the rRABV+GLA-SE group. However, rRABV with and without GLA-SE induced neutralizing titers against WT LBV (B), WT LBV (D), and WT SHIBV.

Overall, LyssaVax stimulates superior titers of VNAs against the phylogroup II viruses tested but has lost some capability in stimulating VNAs against non-RABV phylogroup I viruses. GLA-SE raised the average VNA titer against all viruses tested when administered with both LyssaVax and rRABV.

### Discussion

### Chimeric G Vaccine Rationale

There are a variety of ways to broaden a vaccine's protective breadth, some of which have been attempted for lyssaviruses. A straightforward approach is to create multiple vaccine constructs, each expressing a separate lyssavirus G, but this strategy would multiply the cost of a vaccine already deemed too expensive for the regions that need it most. Furthermore, one lyssavirus G might be immunodominant over others. Some have proposed lyssavirus vaccines in live vectors, which, although less expensive and successful in wildlife RABV vaccination campaigns, are unlikely ever to be approved for use in humans for safety reasons. Another approach is to add multiple Gs to a single vaccine construct. Foreign viral Gs have been successfully added to the RABV genome, and their Gs expressed to comparable amounts as the native RABV G. However, the stability of multiple lyssavirus Gs has not been rigorously tested. Lyssavirus Gs individually exhibit different growth speeds and expression levels. The preliminary data suggest that when combined in a single vector, the less efficient G confers a disadvantage to the virus and puts selective pressure on the virus to lose expression of the G conferring slower growth. Therefore a single-G lyssavirus vaccine construct is preferable.

Prior to the recent publication of the RABV G crystal structure, the protein design effort benefitted the pre-fusion G structure of the related rhabdovirus, VSV. The VSV G structure enabled revisiting the chimera strategy, designing an updated chimeric lyssavirus G, and generating functional virus. Additionally, despite a lack of detailed knowledge about antigenic regions on non-RABV lyssavirus Gs, care was taken to design a chimeric G in which potential antigenic sites were “balanced” between the two major domains (FIG. 11D). Sites II and III are likely of highest importance, because they share binding sites with two of the putative RABV cellular receptors, nicotinic acetyl-choline receptor (nAChR) and the low-affinity neurotrophin receptor (p75NTR), respectively. Although site II has often been considered the most immunogenic based on the high proportions of G-specific mAbs that bind it, many of the mAbs being developed to replace the immune sera in PEP bind to site I. The immunogenicity of site IV has been demonstrated in mice, humans, and dogs. Antibody responses to RABV from different species are not thought to vary significantly. Altogether, it is believed that the domain-based approach to generating chimeric Gs is a superior option.

Glycosylation sites differ between lyssavirus Gs: RABV G has three predicted N-linked glycosylation sites at residues 37, 247, and 319; MOKV G shares the N319 site but has only one other predicted site at N202. Chimeric G 1 therefore has two predicted sites: N202 and N319. The N319 site is conserved across lyssaviruses and is suggested to be the minimal site needed for maturation and trafficking through the endoplasmic reticulum and Golgi apparatus. N37 has been shown not to be efficiently glycosylated and is likely dispensable for proper G folding and function. Finally, it has been suggested that the N202 site in MOKV G is not glycosylated in vivo. Although further study should define the glycosylation sites of the chimeric G, the data are consistent with the cited works because evidence of glycosylation affecting the antigenicity of LyssaVax was not observed.

Recovery of Viruses with Chimeric Gs

It is unclear why the Chimeric G 2 did not enable viral recovery. As the single surface protein, the G carries out numerous tasks, including trimerization, engaging with cellular receptors, and mediating fusion between membranes, any of which may have been disturbed by the newly engineered protein. The immunofluorescence of transfected cells (FIG. 13) demonstrates that Chimeric G 2 is successfully produced, trafficked to the cell sur-face, and exhibits the anticipated antigenicity, suggesting that functional rather than structural issues hampered recovery.

Regardless, Chimeric G 1 is a preferable choice for a chimeric G vaccine because it includes the attenuating mutation R333E within the flap domain contributed by RABV G (FIG. 11E). The R333 residue in RABV G is critical for association with a putative RABV cellular co-receptor, the low-affinity neurotrophin receptor, p75NTR. The R333E mutation alone abrogates pathogenicity by peripheral infection routes in adult mice and likely contributed to Lyssa-Vax's apathogenicity by both routes tested (FIGS. 4A and 4B).

### Vaccine-Generated Antibody Responses

The antibody responses generated against LyssaVax was of particular interest, because antibodies are indicative of a strong vaccine response. LyssaVax elicited high titers of IgG antibodies against both MOKV G and RABV G, as seen by ELISA (FIGS. 5A-5C and FIGS. 14A-14K). Sera from rRABV and rMOKV immunizations also contained appreciable titers of antibodies, which bound to the heterologous antigen (e.g., sera from mouse immunized with rMOKV binding to RABV G) (FIGS. 5A-5C) by day 14 p.i. However, ELISAs detect a wide array of antibodies, regardless of function (e.g., neutralizing and non-neutralizing). Furthermore, the antigens used in the ELISA are detergent solubilized, which may expose epitopes otherwise inaccessible on live, intact virions.

A smaller subset of antibodies function in neutralizing virus, and high titers of these VNAs are the best-established correlate of protection against RABV infection. As such, administration of rabies immune globulin (RIG) is a critical component of current PEP providing short-term passive immunity in addition to a vaccine course. LyssaVax-immune mouse sera neutralized both CVS-11 and MOKV G pseudotypes at nearly the same levels as control immunizations for either rRABV or rMOKV, respectively (FIG. 6 and FIGS. 7A-7E). Although RABV VNAs from LyssaVax were lower than controls at days 28 and 35 (FIG. 6), they were matched by day 56. Furthermore, LyssaVax titers at day 35 averaged over 60-fold higher than the 0.5 IU/mL threshold for protection, demonstrating the robust functionality of the VNAs induced by LyssaVax. Sera from rRABV and rMOKV controls were only marginally cross-neutralizing in the RFFIT and PTV neutralization assay (FIG. 6 and FIGS. 7A-7E), and only by late time points.

After establishing robust functional antibody responses against the component viruses, an immunogenicity study was designed to assess cross-neutralization with additional lyssaviruses. Anticipating the possibility of lower VNA titers against non-component viruses, LyssaVax and the control vaccine, rRABV, were adjuvanted with the Toll-like receptor 4 (TLR4) agonist GLA-SE (FIGS. 9A and 9B). LyssaVax-immune sera neutralized all viruses tested; of phylogroup I viruses, LyssaVax-induced sera neutralized significantly less strongly than that of the rRABV control vaccine and, in the case of DUVV, required GLA-SE to achieve neutralization in the majority of samples. Of phylogroup II viruses, VNA titers induced by LyssaVax+GLA-SE were highest, and in the case of MOKV and LBV D, unadjuvanted LyssaVax was significantly higher than even rRABV+GLA-SE. Two results of the micro-neutralization panel were surprising: the relatively low VNA titers that LyssaVax generated against non-RABV phylogroup I viruses and that rRABV, with and without GLA-SE, induced cross-neutralizing VNAs against LBV-B, LBV-D, and SHIBV.

Regarding low phylogroup I VNA titers, it is possible that antigenic sites located in the core domain, which is contributed by MOKV G in LyssaVax, are more important for neutralizing non-RABV phylogroup I viruses. In a study testing anti-RABV mAbs against a panel of strains and lyssaviruses, none of the five mAbs bound to EBLV-1 or DUVV, and VNA titers against EBLV-1 and DUVV were also lowest in a previously reported RABV G/MOKV G chimeric vaccine. These data suggest that, to provide comprehensive coverage across phylogroups I and II, LyssaVax may need com-ponents from divergent phylogroup I viruses. It has also been shown that higher concentrations of anti-RABV sera are necessary for neutralizing non-RABV phylogroup I viruses, so the RABV G-specific titers from LyssaVax may not have been high enough. The ability of GLA-SE to boost phylogroup I VNA titers when added to LyssaVax supports this.

Although division of the lyssavirus genus into phylogroups is based on genetic and antigenic clustering, there are many examples of less discrete patterns of antigenicity. For example, inter-phylogroup neutralization has been observed in RABV-vaccinated laboratory workers with exceptionally high titers, and there are at least two examples of anti-RABV G mAbs reported to cross-neutralize MOKV: 1409-7 and JA-3.3A5. Furthermore, antigenic cartography studies have determined that only 67% of antigenic differences among lyssavirus Gs are predictable from the amino acid sequence.

In the cases where LyssaVax is cross-neutralizing, it is unknown whether the sera contain individual cross-reactive anti-bodies or whether discrete populations of antibodies bind to each antigen; both possibilities likely contribute. This question can be answered only by isolating and characterizing mAbs, a goal of future studies. Knowledge of where physiologically relevant antibodies bind on non-RABV lyssavirus Gs will be important for detailed study of how LyssaVax elicits protective antibodies against multiple lyssaviruses.

Protection from Rabies Disease

Based on the high titers of VNAs against the two component viruses, which contributed to the chimeric G, full protection was anticipated. LyssaVax indeed protected all mice challenged with either RABV or rMOKV, with no weight loss or clinical symptoms observed. Although lyssaviruses are typically administered intracranially, the i.n. route was chosen in this study for several reasons. First, uniform pathogenicity was observed in female mice during pathogenicity studies (FIGS. 4A and 4B). Second, rMOKV is not pathogenic by the i.m. route (FIG. 4B), consistent with WT MOKV studies. Third, the i.n. route has been shown to be an acceptable alternative to intracranial injection for RABV challenge. Finally, i.n. inoculation poses a lesser risk to laboratory personnel.

Among the control groups, mice immunized and challenged with homologous vaccines/viruses survived, as expected (FIGS. 17C and 17H), whereas some mice survived challenge with heterologous virus (FIGS. 17D and 17G). This was surprising because, despite appreciable titers of antibodies against heterologous Gs detected in the ELISA (FIGS. 5A-5C), mice immunized with rMOKV had marginal RABV-neutralizing titers (FIG. 6) and rRABV did not neutralize MOKV G pseudotypes (FIG. 8). However, in light of the cross-neutralization of other phylogroup I viruses that rMOKV sera exhibited in FIGS. 9A-9B, the survival is less exceptional.

It is notable that two mice immunized with rMOKV lost weight and were euthanized, and two mice immunized with rRABV lost weight after rMOKV challenge and recovered (FIGS. 16A-16H). The atypical challenge model (attenuated strains administered i.n.) may be responsible; this would be addressed by the WT challenge experiment. Given that 9/10 mock-immunized mice were euthanized (FIGS. 16A-16H) and the 10th mouse indeed survived after infection, as evidenced by RABV VNAs detected at necropsy (Table 1, mouse ID 1-4), there is high confidence that the mice in FIGS. 16D and 16G were successfully infected.

The mechanism for developing broadly neutralizing lyssavirus VNAs has not been studied and raises important questions about the antigenic relationships between lyssaviruses and how protection is conferred. There may be additional, uncharacterized immune mechanisms that contribute to protection in the absence of neutralizing antibodies. This possibility warrants further investigation.

### CONCLUSIONS

A lyssavirus vaccine is disclosed featuring a single chimeric glycoprotein that was designed based on observations of predicted lyssavirus G structures. The chimeric G retains antigenic qualities of component Gs (RABV and MOKV) and cell-infecting functionality. When administered as an inactivated vaccine formulation, it stimulates high titers of neutralizing antibodies against component viral Gs and some additional lyssaviruses. Finally, LyssaVax was shown to protect against challenge with RABV and a recombinant MOKV. Development is needed to improve VNA titer responses against phylogroup I viruses. With further development, this vaccine could be employed during a lyssavirus outbreak or supplant current rabies vaccines in areas where non-RABV lyssaviruses are endemic.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### ENUMERATED EMBODIMENTS

Embodiment 1 provides an isolated nucleic acid encoding a recombinant lyssavirus comprising a nucleotide sequence encoding at least a portion of the genome of a rabies virus, wherein the at least a portion of the genome of the rabies virus comprises (a) a nucleotide sequence encoding a rabies virus nucleoprotein (N) or a portion thereof and wherein the recombinant lyssavirus further comprises (b) a nucleotide sequence encoding a glycoprotein (G) or a portion thereof positioned immediately 3′ to the nucleotide sequence encoding the nucleoprotein (N).

Embodiment 2 provides the isolated nucleic acid of embodiment 1, wherein the glycoprotein (G) is selected from a RABV glycoprotein, a MOKV glycoprotein, and a chimeric MOKV/RABV glycoprotein.

Embodiment 3 provides the isolated nucleic acid of embodiment 1 or 2, wherein the recombinant lyssavirus is a SADB-19 rabies virus strain.

Embodiment 4 provides the isolated nucleic acid of embodiment 2 or 3, wherein the nucleotide sequence encoding the RABV glycoprotein, the chimeric MOKV/RABV glycoprotein, or a portion thereof comprises a mutation that results in insertion of glutamic acid in place of arginine at position 333 of the RABV glycoprotein.

Embodiment 5 provides the isolated nucleic acid of any one of embodiments 2-4, wherein the nucleotide sequence encoding the chimeric MOKV/RABV glycoprotein or a portion thereof comprises a nucleotide sequence encoding a RABV clip domain, a nucleotide sequence encoding a MOKV core domain, and a nucleotide sequence encoding a RABV flap domain.

Embodiment 6 provides the isolated nucleic acid of embodiment 2 or 3, wherein the nucleotide sequence encoding the chimeric MOKV/RABV glycoprotein or a portion thereof comprises a nucleotide sequence encoding a MOKV clip domain, a nucleotide sequence encoding a RABV core domain, and a nucleotide sequence encoding a MOKV flap domain.

Embodiment 7 provides the isolated nucleic acid of any one of embodiments 1-6, wherein the nucleotide sequence (b) encoding the glycoprotein (G) is positioned immediately 5′ to (c) a nucleotide sequence encoding a rabies virus phosphoprotein (P).

Embodiment 8 provides the isolated nucleic acid of any one of embodiments 1-7, wherein the nucleic acid comprises a nucleotide sequence having at least 85% sequence identity to SEQ ID NO: 1, at least 85% sequence identity to SEQ ID NO: 2, or at least 85% sequence identity to SEQ ID NO: 4.

Embodiment 9 provides the isolated nucleic acid of any one of embodiments 1-8, wherein the nucleic acid comprises a nucleotide sequence having at least 90% sequence identity to SEQ ID NO: 1, at least 90% sequence identity to SEQ ID NO: 2, or at least 90% sequence identity to SEQ ID NO: 4.

Embodiment 10 provides the isolated nucleic acid of any one of embodiments 1-9, wherein the nucleic acid comprises a nucleotide sequence having at least 95% sequence identity to SEQ ID NO: 1, at least 95% sequence identity to SEQ ID NO: 2, or at least 95% sequence identity to SEQ ID NO: 4.

Embodiment 11 provides the isolated nucleic acid of any one of embodiments 1-10, wherein the nucleic acid comprises a nucleotide sequence having at least 99% sequence identity to SEQ ID NO: 1, at least 99% sequence identity to SEQ ID NO: 2, or at least 99% sequence identity to SEQ ID NO: 4.

Embodiment 12 provides the isolated nucleic acid of any one of embodiments 1-11, wherein the nucleic acid comprises the nucleotide sequence set forth in SEQ ID NO: 1, SEQ ID NO:2, or SEQ ID NO: 4.

Embodiment 13 provides the isolated nucleic acid of any one of embodiments 1-12, wherein the nucleic acid encodes a recombinant rabies virus.

Embodiment 14 provides the isolated nucleic acid comprising (a) a nucleotide sequence encoding a rabies virus nucleoprotein (N) or a portion thereof and (b) a nucleotide sequence encoding a glycoprotein (G) or a portion thereof positioned immediately 3′ to the nucleotide sequence encoding the nucleoprotein (N).

Embodiment 15 provides the isolated nucleic acid of embodiment 14, wherein the glycoprotein (G) is selected from a RABV glycoprotein, a MOKV glycoprotein, and a chimeric MOKV/RABV glycoprotein.

Embodiment 16 provides the isolated nucleic acid of embodiment 15, wherein the nucleotide sequence encoding the RABV glycoprotein, the chimeric MOKV/RABV glycoprotein, or portion thereof comprises a mutation that results in insertion of glutamic acid in place of arginine at position 333 of the RABV glycoprotein.

Embodiment 17 provides the isolated nucleic acid of embodiment 15 or 16, wherein the nucleotide sequence encoding the chimeric MOKV/RABV glycoprotein or a portion thereof comprises a nucleotide sequence encoding a RABV clip domain, a nucleotide sequence encoding a MOKV core domain, and a nucleotide sequence encoding a RABV flap domain.

Embodiment 18 provides the isolated nucleic acid of embodiment 15, wherein the nucleotide sequence encoding the chimeric MOKV/RABV glycoprotein or a portion thereof comprises a nucleotide sequence encoding a MOKV clip domain, a nucleotide sequence encoding a RABV core domain, and a nucleotide sequence encoding a MOKV flap domain.

Embodiment 19 provides the isolated nucleic acid of any one of embodiments 15-18, wherein the nucleotide sequence encoding the RABV glycoprotein, the MOKV glycoprotein, the chimeric MOKV/RABV glycoprotein, or portion thereof is positioned immediately 5′ to (c) a sequence nucleotide encoding a rabies virus phosphoprotein (P).

Embodiment 20 provides the isolated nucleic acid of embodiment 19, wherein the nucleotide sequence encoding the rabies virus phosphoprotein (P) is positioned immediately 5′ to (d) a nucleotide sequence encoding a rabies virus protein (M) and wherein the nucleotide sequence encoding protein (M) is positioned immediately 5′ to (e) a nucleotide sequence encoding a rabies virus protein (L).

Embodiment 21 provides the isolated nucleic acid of any one of embodiments 14-20, wherein the nucleic acid comprises a nucleotide sequence having at least 85% sequence identity to SEQ ID NO: 1, at least 85% sequence identity to SEQ ID NO: 2, or at least 85% sequence identity to SEQ ID NO: 4.

Embodiment 22 provides the isolated nucleic acid of any one of embodiments 14-21, wherein the nucleic acid comprises a nucleotide sequence having at least 90% sequence identity to SEQ ID NO: 1, at least 90% sequence identity to SEQ ID NO: 2, or at least 90% sequence identity to SEQ ID NO: 4.

Embodiment 23 provides the isolated nucleic acid of any one of embodiments 14-22, wherein the nucleic acid comprises a nucleotide sequence having at least 95% sequence identity to SEQ ID NO: 1, at least 95% sequence identity to SEQ ID NO: 2, or at least 95% sequence identity to SEQ ID NO: 4.

Embodiment 24 provides the isolated nucleic acid of any one of embodiments 14-23, wherein the nucleic acid comprises a nucleotide sequence having at least 99% sequence identity to SEQ ID NO: 1, at least 99% sequence identity to SEQ ID NO: 2, or at least 99% sequence identity to SEQ ID NO: 4.

Embodiment 25 provides the isolated nucleic acid of any one of embodiments 14-24, wherein the nucleic acid comprises the nucleotide sequence set forth in SEQ ID NO: 1, SEQ ID NO: 2, or SEQ ID NO: 4.

Embodiment 26 provides the isolated nucleic acid of any one of embodiments 14-25, wherein the nucleic acid encoding the recombinant virus is codon optimized for expression in a host cell.

Embodiment 27 provides the isolated nucleic acid of embodiment 26, wherein the host cell is a mammalian cell.

Embodiment 28 provides a recombinant virus encoded by a nucleic acid sequence comprising at least a portion of the genome of the rabies virus, wherein the at least a portion of the genome of the rabies virus comprises (a) a nucleotide sequence encoding a rabies virus nucleoprotein (N) or a portion thereof and wherein the nucleic acid sequence further comprises (b) a nucleotide sequence encoding a glycoprotein (G) or a portion thereof positioned immediately 3′ to the nucleotide sequence encoding the nucleoprotein (N).

Embodiment 29 provides the recombinant virus of embodiment 28, wherein the glycoprotein (G) is selected from a RABV glycoprotein, a MOKV glycoprotein, and a chimeric MOKV/RABV glycoprotein.

Embodiment 30 provides the recombinant virus of embodiment 29, wherein the nucleotide sequence encoding the RABV glycoprotein, the chimeric MOKV/RABV glycoprotein, or portion thereof comprises a mutation that results in insertion of glutamic acid in place of arginine at position 333 of the RABV glycoprotein.

Embodiment 31 provides the recombinant virus of embodiment 29 or 30, wherein the nucleotide sequence encoding the chimeric MOKV/RABV glycoprotein or portion thereof comprises (a) nucleotide sequence encoding a RABV clip domain, (b) a nucleotide sequence encoding a MOKV core domain and (c) a nucleotide sequence encoding a RABV flap domain.

Embodiment 32 provides the recombinant virus of embodiment 29, wherein the nucleotide sequence encoding the chimeric MOKV/RABV glycoprotein or a portion thereof comprises (a) nucleotide sequence encoding a MOKV clip domain, (b) a nucleotide sequence encoding a RABV core domain, and (c) a nucleotide sequence encoding a MOKV flap domain.

Embodiment 33 provides the recombinant virus of any of embodiments 28-32, wherein the recombinant virus is a recombinant rabies virus.

Embodiment 34 provides a recombinant virus encoded by a nucleic acid of any one of embodiments 1-27.

Embodiment 35 provides a vector comprising the nucleic acid of any one of embodiments 1-27.

Embodiment 36 provides a vaccine comprising the recombinant virus of any one of embodiments 28-34 or a recombinant virus encoded by the isolated nucleic acid of any one of embodiments 1-27, and a pharmaceutically acceptable carrier.

Embodiment 37 provides the vaccine of embodiment 36, further comprising an adjuvant.

Embodiment 38 provides the vaccine of embodiment 36 or 37, wherein the virus is deactivated.

Embodiment 39 provides a method of generating an immune response against a lyssavirus in a subject in need thereof, the method comprising administering to the subject an effective amount of the recombinant virus of any one of embodiments 28-34, a recombinant virus encoded by the isolated nucleic acid of any one of embodiments 1-27, or the vaccine of any one of embodiments 36-38.

Embodiment 40 provides a method of vaccinating a subject against a lyssavirus, the method comprising administering to the subject an effective amount of the recombinant virus of any one of embodiments 28-34, a recombinant virus encoded by the isolated nucleic acid of any one of embodiments 1-27, or the vaccine of any one of embodiments 36-38.

Embodiment 41 provides a method of providing immunity against a lyssavirus in a subject, the method comprising administering to the subject an effective amount of the recombinant virus of any one of embodiments 28-34, a recombinant virus encoded by the isolated nucleic acid of any one of embodiments 1-27, or the vaccine of any one of embodiments 36-38.

Embodiment 42 provides a method of treating and/or preventing a disease or disorder associated with a lyssavirus in a subject in need thereof, the method comprising administering to the subject an effective amount of the recombinant virus of any one of embodiments 28-34, a recombinant virus encoded by the isolated nucleic acid of any one of embodiments 1-27, or the vaccine of any one of embodiments 36-38.

Embodiment 43 provides a method of increasing immunogenicity against a lyssavirus in a subject in need thereof, the method comprising administering to the subject an effective amount of the recombinant virus of any one of embodiments 28-34, a recombinant virus encoded by the isolated nucleic acid of any one of embodiments 1-27, or the vaccine of any one of embodiments 36-38.

Embodiment 44 provides the method of any one of embodiments 39-43, wherein the subject is a mammal.

Embodiment 45 provides the method of any one of embodiments 39-44, wherein the lyssavirus is a rabies virus.

Embodiment 46 provides a method of increasing expression of a recombinant lyssavirus in a host cell, the method comprising expressing in the host cell a nucleic acid sequence of any one of embodiments 1-27.

Embodiment 47 provides the method of embodiment 46, wherein the host cell is a mammalian cell.

Embodiment 48 provides the method of embodiment 46 or 47, wherein the recombinant lyssavirus is a recombinant rabies virus.

1. An isolated nucleic acid encoding a recombinant lyssavirus comprising a nucleotide sequence encoding at least a portion of the genome of a rabies virus, wherein the at least a portion of the genome of the rabies virus comprises (a) a nucleotide sequence encoding a rabies virus nucleoprotein (N) or a portion thereof and wherein the recombinant lyssavirus further comprises (b) a nucleotide sequence encoding a glycoprotein (G) or a portion thereof positioned immediately 3′ to the nucleotide sequence encoding the nucleoprotein (N).

2. The isolated nucleic acid of claim 1, wherein the glycoprotein (G) is selected from a RABV glycoprotein, a MOKV glycoprotein, and a chimeric MOKV/RABV glycoprotein.

3. The isolated nucleic acid of claim 1, wherein the recombinant lyssavirus is a SADB-19 rabies virus strain.

4. The isolated nucleic acid of claim 2, wherein the nucleotide sequence encoding the RABV glycoprotein, the chimeric MOKV/RABV glycoprotein, or a portion thereof comprises a mutation that results in insertion of glutamic acid in place of arginine at position 333 of the RABV glycoprotein.

5. The isolated nucleic acid of claim 2, wherein the nucleotide sequence encoding the chimeric MOKV/RABV glycoprotein or a portion thereof comprises a nucleotide sequence encoding a RABV clip domain, a nucleotide sequence encoding a MOKV core domain, and a nucleotide sequence encoding a RABV flap domain.

6. The isolated nucleic acid of claim 2, wherein the nucleotide sequence encoding the chimeric MOKV/RABV glycoprotein or a portion thereof comprises a nucleotide sequence encoding a MOKV clip domain, a nucleotide sequence encoding a RABV core domain, and a nucleotide sequence encoding a MOKV flap domain.

7. The isolated nucleic acid of claim 1, wherein the nucleotide sequence (b) encoding the glycoprotein (G) is positioned immediately 5′ to (c) a nucleotide sequence encoding a rabies virus phosphoprotein (P).

8. The isolated nucleic acid of claim 1, wherein the nucleic acid comprises a nucleotide sequence having at least 85% sequence identity to SEQ ID NO: 1, at least 85% sequence identity to SEQ ID NO: 2, or at least 85% sequence identity to SEQ ID NO: 4.

9. The isolated nucleic acid of claim 1, wherein the nucleic acid comprises a nucleotide sequence having at least 90% sequence identity to SEQ ID NO: 1, at least 90% sequence identity to SEQ ID NO: 2, or at least 90% sequence identity to SEQ ID NO: 4.

10. The isolated nucleic acid of claim 1, wherein the nucleic acid comprises a nucleotide sequence having at least 95% sequence identity to SEQ ID NO: 1, at least 95% sequence identity to SEQ ID NO: 2, or at least 95% sequence identity to SEQ ID NO: 4.

11. The isolated nucleic acid of claim 1, wherein the nucleic acid comprises a nucleotide sequence having at least 99% sequence identity to SEQ ID NO: 1, at least 99% sequence identity to SEQ ID NO: 2, or at least 99% sequence identity to SEQ ID NO: 4.

12. The isolated nucleic acid of claim 1, wherein the nucleic acid comprises the nucleotide sequence set forth in SEQ ID NO: 1, SEQ ID NO:2, or SEQ ID NO: 4.

13. The isolated nucleic acid of claim 1, wherein the nucleic acid encodes a recombinant rabies virus.

14. An isolated nucleic acid comprising (a) a nucleotide sequence encoding a rabies virus nucleoprotein (N) or a portion thereof and (b) a nucleotide sequence encoding a glycoprotein (G) or a portion thereof positioned immediately 3′ to the nucleotide sequence encoding the nucleoprotein (N).

15. The isolated nucleic acid of claim 14, wherein the glycoprotein (G) is selected from a RABV glycoprotein, a MOKV glycoprotein, and a chimeric MOKV/RABV glycoprotein.

16. The isolated nucleic acid of claim 15, wherein the nucleotide sequence encoding the RABV glycoprotein, the chimeric MOKV/RABV glycoprotein, or portion thereof comprises a mutation that results in insertion of glutamic acid in place of arginine at position 333 of the RABV glycoprotein.

17. The isolated nucleic acid of claim 15, wherein the nucleotide sequence encoding the chimeric MOKV/RABV glycoprotein or a portion thereof comprises a nucleotide sequence encoding a RABV clip domain, a nucleotide sequence encoding a MOKV core domain, and a nucleotide sequence encoding a RABV flap domain.

18. The isolated nucleic acid of claim 15, wherein the nucleotide sequence encoding the chimeric MOKV/RABV glycoprotein or a portion thereof comprises a nucleotide sequence encoding a MOKV clip domain, a nucleotide sequence encoding a RABV core domain, and a nucleotide sequence encoding a MOKV flap domain.

19. The isolated nucleic acid of claim 15, wherein the nucleotide sequence encoding the RABV glycoprotein, the MOKV glycoprotein, the chimeric MOKV/RABV glycoprotein, or portion thereof is positioned immediately 5′ to (c) a sequence nucleotide encoding a rabies virus phosphoprotein (P).

20. The isolated nucleic acid of claim 19, wherein the nucleotide sequence encoding the rabies virus phosphoprotein (P) is positioned immediately 5′ to (d) a nucleotide sequence encoding a rabies virus protein (M) and wherein the nucleotide sequence encoding protein (M) is positioned immediately 5′ to (e) a nucleotide sequence encoding a rabies virus protein (L).

21. The isolated nucleic acid of claim 14, wherein the nucleic acid comprises a nucleotide sequence having at least 85% sequence identity to SEQ ID NO: 1, at least 85% sequence identity to SEQ ID NO: 2, or at least 85% sequence identity to SEQ ID NO: 4.

22. The isolated nucleic acid of claim 14, wherein the nucleic acid comprises a nucleotide sequence having at least 90% sequence identity to SEQ ID NO: 1, at least 90% sequence identity to SEQ ID NO: 2, or at least 90% sequence identity to SEQ ID NO: 4.

23. The isolated nucleic acid of claim 14, wherein the nucleic acid comprises a nucleotide sequence having at least 95% sequence identity to SEQ ID NO: 1, at least 95% sequence identity to SEQ ID NO: 2, or at least 95% sequence identity to SEQ ID NO: 4.

24. The isolated nucleic acid of claim 14, wherein the nucleic acid comprises a nucleotide sequence having at least 99% sequence identity to SEQ ID NO: 1, at least 99% sequence identity to SEQ ID NO: 2, or at least 99% sequence identity to SEQ ID NO: 4.

25. The isolated nucleic acid of claim 14, wherein the nucleic acid comprises the nucleotide sequence set forth in SEQ ID NO: 1, SEQ ID NO: 2, or SEQ ID NO: 4.

26. The isolated nucleic acid of claim 14, wherein the nucleic acid encoding the recombinant virus is codon optimized for expression in a host cell.

27. The isolated nucleic acid of claim 26, wherein the host cell is a mammalian cell.

28. A recombinant virus encoded by a nucleic acid sequence comprising at least a portion of the genome of the rabies virus, wherein the at least a portion of the genome of the rabies virus comprises (a) a nucleotide sequence encoding a rabies virus nucleoprotein (N) or a portion thereof and wherein the nucleic acid sequence further comprises (b) a nucleotide sequence encoding a glycoprotein (G) or a portion thereof positioned immediately 3′ to the nucleotide sequence encoding the nucleoprotein (N).

29. The recombinant virus of claim 28, wherein the glycoprotein (G) is selected from a RABV glycoprotein, a MOKV glycoprotein, and a chimeric MOKV/RABV glycoprotein.

30. The recombinant virus of claim 29, wherein the nucleotide sequence encoding the RABV glycoprotein, the chimeric MOKV/RABV glycoprotein, or portion thereof comprises a mutation that results in insertion of glutamic acid in place of arginine at position 333 of the RABV glycoprotein.

31. The recombinant virus of claim 29, wherein the nucleotide sequence encoding the chimeric MOKV/RABV glycoprotein or portion thereof comprises (a) nucleotide sequence encoding a RABV clip domain, (b) a nucleotide sequence encoding a MOKV core domain and (c) a nucleotide sequence encoding a RABV flap domain.

32. The recombinant virus of claim 29, wherein the nucleotide sequence encoding the chimeric MOKV/RABV glycoprotein or a portion thereof comprises (a) nucleotide sequence encoding a MOKV clip domain, (b) a nucleotide sequence encoding a RABV core domain, and (c) a nucleotide sequence encoding a MOKV flap domain.

33. The recombinant virus of claim 28, wherein the recombinant virus is a recombinant rabies virus.

34. A recombinant virus encoded by a nucleic acid of claim 1.

35. A vector comprising the nucleic acid of any one of claim 1.

36. A vaccine comprising the recombinant virus of claim 1, and a pharmaceutically acceptable carrier.

37. The vaccine of claim 36, further comprising an adjuvant.

38. The vaccine of claim 36, wherein the virus is deactivated.

39. A method of generating an immune response against a lyssavirus in a subject in need thereof, the method comprising administering to the subject an effective amount of the recombinant virus of claim 28.

40. A method of vaccinating a subject against a lyssavirus, the method comprising administering to the subject an effective amount of the recombinant virus of claim 28.

41. A method of providing immunity against a lyssavirus in a subject, the method comprising administering to the subject an effective amount of the recombinant virus of claim 28.

42. A method of treating and/or preventing a disease or disorder associated with a lyssavirus in a subject in need thereof, the method comprising administering to the subject an effective amount of the recombinant virus of claim 28.

43. A method of increasing immunogenicity against a lyssavirus in a subject in need thereof, the method comprising administering to the subject an effective amount of claim 28.

44. The method of claim 39, wherein the subject is a mammal.

45. The method of claim 39, wherein the lyssavirus is a rabies virus.

46. A method of increasing expression of a recombinant lyssavirus in a host cell, the method comprising expressing in the host cell a nucleic acid sequence of claim 1.

47. The method of claim 46, wherein the host cell is a mammalian cell.

48. The method of claim 46, wherein the recombinant lyssavirus is a recombinant rabies virus.
